(19) United States
(12) Patent Application Publication
(10) Pub. No.: US 2026/0201342 A1
(43) Pub. Date: Jul. 16, 2026

(54) METHODS, DEVICES AND SYSTEMS OF PRODUCING CULTURED TISSUES

(71) Applicant: NUTRIR LIMITED, Hong Kong (CN)

(72) Inventor: Yu Ka CHAN, Hong Kong (CN)

(21) Appl. No.: 19/136,280

(22) PCT Filed: Dec. 7, 2023
(86) PCT No.: PCT/CN2023/137064

§ 371 Date: Jun. 5, 2025

Related U.S. Application Data
(60) Provisional application No. 63/386,495, filed on Dec. 7, 2022.

(51) Int. Cl. C12N 5/071; A23L 13/00; C12M 3/00; C12M 3/06; C12N 5/077

(52) U.S. Cl. CPC C12N 5/0697; A23L 13/00; C12M 21/08; C12M 23/16; C12N 5/0658; C12N 2533/54

(57) ABSTRACT

It provides the methods, devices and systems of producing cultured tissue. The methods comprise the steps of (a) providing a hydrogel composition and solidifying the hydrogel composition to form a hydrogel portion, wherein the hydrogel portion comprises a plurality of first channels in a predetermined first array; (b) providing at least one first cell composition to individual first channel to form at least one first cell culture therein, such that a tissue construct is formed. The methods, devices and systems produce cultured meat with controlled distributions of tissue components, thus the texture of cultured tissue similar to natural tissue.

### CROSS-REFERENCE TO RELATED APPLICATION

This application claims priority to, and the benefits of, U.S. Provisional Application having Ser. No. 63/386,495 filed on Dec. 7, 2022. The entire contents of the foregoing application are hereby incorporated by reference in its entirety for all purposes.

### FIELD OF INVENTION

This application relates to methods, devices and systems of producing cultured cells. In particular, this application relates to methods, devices and systems of producing cultured tissues such as cultured meat.

### BACKGROUND OF INVENTION

A major component of meat is the muscle fibers and intra/intermuscular fats. Attempts have been made to produce cultured meat that can mimic meat structurally, but challenges have been encountered. For example, there are difficulties in creating meat like texture due to low resolution of 3D printers, or the controllability of tissue architecture in a three-dimensional manner in the case of cell-sheet technologies. There are also difficulties to produce thick cultured meat structures while maintaining tissue viability due to the lack of vasculature. There is a great need to produce cultured meat with meat-like texture similar to natural meat and to provide vasculature.

### SUMMARY OF INVENTION

Disclosed herein are devices, systems and methods that are useful for producing cultured tissues.

In some embodiments, provided is a method of producing cultured tissue, including the steps of: (a) providing a hydrogel composition and solidifying the hydrogel composition to form a hydrogel portion, wherein the hydrogel portion includes a plurality of first channels in a predetermined first array; (b) providing at least one first cell composition to individual first channel to form at least one first cell culture therein, such that that a tissue construct is formed.

In some embodiments, provided is a device of producing cultured tissue including a plurality of first channel forming units, individual first channel forming unit having a first diameter; and a holder assembly which include a holder unit and at least one supporting unit, each including a plurality of first channel receiving portions, wherein individual first channel receiving portion is sized and shaped to receive at least a portion of an individual first channel forming unit, wherein the plurality of first channel receiving portions are constructed and arranged in a first pattern such that the plurality of first channel forming units can be assembled with the holder assembly to form a predetermined first array and a remaining space in the holder assembly, and wherein at least a portion of the remaining space is configured to receive a hydrogel composition to form a solidified, hydrogel portion, such that a plurality of first channels in the predetermined, first array are formed by removing the plurality of first channel forming units from the hydrogel portion, individual first channel is configured to receive at least one first cell composition therein to form at least one first cell culture, thereby a tissue construct is produced.

In some embodiments, provided is a system of producing cultured tissue, including a device as described in any one of the embodiments herein to produce a hydrogel construct having a plurality of third channels; a hydrogel construct adapter, configured to connect the hydrogel construct with at least one tubing; a mixer for oxygenating a medium; at least one pump system for circulating the medium from the mixer to the hydrogel construct through the plurality of third channels; and optionally a medium recycler to remove any waste.

In some embodiments, provided is a cultured tissue prepared by any one of the methods as described in any one of the embodiments herein, wherein the cultured tissues are derived from animals selected from the group consisting of mammals, birds, fish, invertebrates, reptiles, and amphibians.

### Advantages of the Present Disclosure

There are many advantages provided by the present invention. In certain embodiments, the provided devices, systems and methods can produce cultured meat with aligned and controlled thin (e.g., <100 μm) diameters of muscle fibers. In certain embodiments, the provided devices, systems and methods can produce cultured meat with controlled inter-fiber distance (e.g., less than 20 μm).

In certain embodiments, the provided devices, systems and methods can produce cultured meat with full controllability of tissue architecture in a three-dimensional manner. The distribution of muscle and fat regions can also be controlled therefore improving texture of cultured meat. When all dimensions are optimized (channel diameter, distance between two adjacent channels, and specific cells seeded into specific channels), the produced cultured meat can be structurally similar to natural meat which no other method has been able to achieve. And when exercised, the texture of the cultured meat can be even closer to that of natural meat.

In certain embodiments, the cultured meat may contain fats that give marbling and flavor, muscle that is the fundamental part of meat, fibroblasts as the structural cells and/or endothelial cells for blood vessels.

In certain embodiments, provided devices, systems and methods can produce cultured meat with controlled distributions of tissue components (such as muscle fibers, intramuscular fats etc), thus improving the texture of cultured tissue (such as cultured meat) similar to natural tissue (such as natural meat, e.g., beef or pork).

In some embodiments, the tissue constructs are configured to have final dimensions similar to that in the desired meat (e.g. fat fiber diameter, fat fiber length, fiber-fiber distance etc).

In some embodiments, the distance between two adjacent third channels is configured to be small enough to allow for a cell viability of at least certain degree percentage in the final meat construct, for example, at least 90%, 80%, 70%, 60%, 50%, 40%, 30%, 20%, 10%, or 5%.

In some embodiments, the provided devices, systems and methods of creating tissue construct such as meat is highly customizable and flexible. By simply increasing the number of needles, the needle lengths, and needle distances, more tissue construct such as meat can be produced. In some embodiments, by utilizing high-resolution laser cutting, metal plates (supporting units) of multiple arrangements of holes (channel receiving portions) can be created to create meat of different designs, and different textures. This invention is highly beneficial in facilitating the creation of texture that mimics natural meat, or even create new textures that are non-existent in natural meat.

In some embodiments, hydrogel portion holds the channels within the tissue constructs in place.

In some embodiments, hydrogel portion serves as an extracellular matrix mimic. Different meat muscle fibers can have different extracellular matrix (e.g. endomysium distance which is the area between muscle-fiber to fiber within a fascicle; and perimysium distance which is the sheath that covers a fascicle) thickness. In some embodiments, provided hydrogel portion effectively mimics these distances given that the plate (supporting unit) layout is well designed, and if shrinking is properly performed on the entire construct.

In some embodiments, the provided devices, systems and methods of creating tissue construct promote consistent microchannels formation within hydrogels. In some embodiments, the provided devices, systems and methods of creating tissue construct allow for multiple designs of plates, and thus designs of microchannel arrangements, to be created.

In some embodiments, the provided devices, systems and methods of creating tissue construct support the formation of muscle-fibres, as when at least one cell composition is injected into the densely packed microchannels, densely packed muscle-fibres can be formed as a result.

### BRIEF DESCRIPTION OF FIGURES

FIG. 1 is a schematic cross-sectional illustration of an example channel array mimicking the dimensions of a section of an in-vivo Wagyu tissue, according to an embodiment.

FIG. 2 is a schematic cross-sectional illustration of another example channel array, according to an embodiment.

FIG. 3A is a schematic illustration showing an example support plate as described in Example 3.

FIGS. 3B-3C are schematic illustration and a zoomed-in view of an example channel assembly according to Example 3.

FIG. 4A is a schematic illustration showing an example channel assembly filled with a hydrogel composition to form a hydrogel portion, according to Example 4.

FIG. 4B is a schematic illustration showing an example channel assembly having the hydrogel portion with a plurality of first channel forming units removed from the channel assembly, according to Example 4.

FIGS. 4C and 4D are schematic illustration and a cross-sectional view of an example tissue construct according to Example 4.

FIGS. 5A-5C are schematic illustration and cross-sectional views of an example tissue construct extruded out with a plurality of third channels, according to Example 5.

FIGS. 6A and 6B are schematic illustrations of an example tissue construct adapter and tissue construct assembly, according to Example 6.

FIG. 6C is a schematic illustration showing an example perfusion assembly system, according to Example 6.

FIGS. 7A-7C are schematic illustration and cross-sectional views of an example tissue construct after undergoing a shrinking step, according to Example 7.

FIG. 8A is a flow chart of an example method of producing cultured tissues.

FIG. 8B is a flow chart of another example method of producing cultured tissues.

FIG. 9 is a schematic cross-sectional illustration of another example channel array mimicking the dimensions of a section of an in-vivo Wagyu or Angus cow tissue, according to Example 10.

FIG. 10 is a schematic cross-sectional illustration of another example channel array, according to Example 10.

FIG. 11A is a schematic illustration showing an example support plate as described in Example 11.

FIG. 11B is a schematic illustration showing an example channel assembly according to Example 11.

FIG. 11C is a schematic zoomed-in view of an example channel assembly according to Example 11.

FIG. 12A is a schematic illustration showing an example channel assembly filled with a hydrogel composition to form a hydrogel portion, according to Example 12.

FIG. 12B is a schematic illustration showing an example channel assembly having the hydrogel portion with a plurality of first channel forming units removed from the channel assembly, according to Example 12.

FIGS. 12C and 12D are schematic illustration and a cross-sectional view of an example tissue construct according to Example 12.

FIGS. 13A-13B are schematic illustration and cross-sectional views of an example tissue construct extruded out with a plurality of third channels, according to Example 13.

FIGS. 14A and 14B are schematic illustrations of an example tissue construct adapter and tissue construct assembly, according to Example 14.

FIGS. 14C-14E are schematic illustrations showing the example tissue construct adapter as described in Example 14 at different perspectives.

FIG. 14F is a schematic illustration showing an example perfusion assembly system, according to Example 14.

FIGS. 15A-15C are schematic illustration and cross-sectional views of an example tissue construct after undergoing a shrinking step, according to Example 7.

FIGS. 16A-16C are schematic illustrations showing another example channel assembly according to Example 16.

FIGS. 17A-17B are schematic illustrations showing another example channel assembly according to Example 17.

FIG. 17C is a microscopic image showing a microchannel seeded with myoblasts in the presence of fibrinogen thrombin hydrogel.

FIG. 17D is a microscopic image showing the aligned muscle-fibers parallel to the microchannel.

FIG. 17E is a microscopic image showing the aligned muscle-fibers parallel to the microchannel.

FIG. 17F is a microscopic image showing a microchannel seeded with myoblasts without the presence of hydrogels.

FIGS. 18A-18C are schematic illustrations showing another example channel according to Example 18.

FIG. 18D is a photograph showing an example trimmed tissue construct prepared using Formulation #2 as hydrogel portion.

FIG. 18E is a photograph showing an example untrimmed tissue construct prepared using Formulation #3 as hydrogel portion.

FIG. 18F is a photograph showing an example untrimmed tissue construct prepared using Formulation #1 as hydrogel portion.

FIGS. 19A-19C are schematic illustrations showing another example channel assembly according to Example 19.

FIGS. 19D-19F are photographs showing the isometric view, front cross-sectional view and side-view of an example tissue construct prepared using Formulation #1 as hydrogel portion, according to Example 19.

FIGS. 19G-19H are photographs showing the isometric view and side-view of an example tissue construct prepared using Formulation #3 as hydrogel portion, according to Example 19.

FIGS. 19I-19K are photographs showing the isometric view, front view and side-view of another example tissue construct prepared using Formulation #2 as hydrogel portion, according to Example 19.

FIG. 19L is a microscopic image showing porcine myoblasts in Formulation #6 incorporated into the microchannels, according to Example 19.

FIGS. 20A-20C are schematic front-view illustrations of the process of using an example device, according to Example 20.

FIGS. 20D-20F are schematic isometric of the example device according to Example 20.

FIGS. 21A-21B are schematic illustrations of an example tissue construct, according to Example 21, in the front view and isometric view, respectively.

FIGS. 21C-21D are schematic illustrations of an example tissue construct after the hydrogel portion was dissolved, according to Example 21, in the front view and isometric view, respectively.

### DETAILED DESCRIPTION

Although the description referred to particular embodiments, the disclosure should not be construed as limited to the embodiments set forth herein.

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For the sake of clarity, “comprising”, “including”, “containing” and “having”, and any related forms are open-ended terms which allows for additional elements or features beyond the named essential elements, whereas “consisting of” is a closed end term that is limited to the elements recited in the claim and excludes any element, step, or ingredient not specified in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than +20% of a stated value. By way of example only, about 50 means from 40 to 60 including all values in between. As used herein, the phrase “about” a specific value also includes the specific value, for example, about 50 includes 50.

As used herein and in the claims, the terms “general” or “generally”, or “substantial” or “substantially” mean that the recited characteristic, angle, shape, state, structure, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As used herein, the terms “cultured-meat”, “cultivated-meat”, “in-vitro meat”, “cell-based meat”, “clean meat”, “synthetic meat”, and “lab-grown meat” are used interchangeably referring to a meat product comprising tissues derived from cell culturing. In some examples, cultured-meat is produced by culturing animal cells or tissues in vitro without growing the entire animal (also known as ‘cellular agriculture’). For example, the process involves isolating meat-specific cells from an animal, an expansion step where cells are grown in a nutrient-rich medium for cells to proliferate, followed by final harvesting of cells into the final meat product.

As used herein, the terms “proliferation” or “expansion” refers to the process of increasing cell number or cell population by culturing the target cells.

As used herein, the term “culture medium” refers to a composition or formulation to support growth and/or other cellular activities of target cells or cell tissues.

As used herein, the term “basal medium” or “minimal medium” refers to a growth medium that provides essential nutrients for survival and cultivation of desired cells. Basal medium may serve as a starting point or foundation upon which specific additives can be added to meet the specific requirements of the desired cell culture. Examples thereof include but not limited to Dulbecco's Modified Eagle Medium (DMEM), Ham's F12 medium (F12), Dulbecco's Modified Eagle Medium F12 (DMEM: F12), Ham's F10 medium (F10), Roswell Park Memorial Institute 1640 Medium (RPMI 1640), Minimum Essential Medium (MEM), serum free media (SFM), or combination thereof.

As used herein, the term “final concentration” refers to the concentration when a substance or a component is present in a solution or a mixture (such as a hydrogel composition or cell composition) after all necessary dilutions or reactions have taken place. In some examples, the total concentration of the hydrogel composition or cell composition is calculated in weight by volume (100% (w/v)). In some other examples, the total concentration of the hydrogel composition or cell composition is calculated in volume by volume (100% (v/v)).

As used herein, the term “animals” refers to eukaryote species such as mammalians (such as porcine, bovine, ovine, equine, canine, feline, rodent), birds or avian, reptile, fish, amphibians, crustaceans, mollusk, cephalopods or the like, etc.

As used herein, the term “perfusion” refers to a controlled delivery of at least one fluid to support oxygenation, nutrition, and/or waste removal. As an example, oxygenated medium can be perfused through a hydrogel construct to support cell growth and cell differentiation, using a perfusion assembly system.

As used herein, the terms “device” or “channel assembly” are used interchangeably when referring to an apparatus that can be used for producing final products or intermediates of cultured tissues or tissue constructs such as cultured meat.

As used herein, the term “supporting unit” refers to a component of the device having a plurality of channel receiving portions to support and define the relative position of at least a portion of an individual channel forming unit. In some embodiments, the supporting unit is or contains one or more plates having multiple holes as the channel receiving portions in specific patterns. In some embodiments, the supporting unit is in the form of a plate. In some other embodiments, the supporting unit is in the form of a block with a larger thickness to support larger portion of a channel.

As used herein, the terms “pattern” and “channel array” refer to the arrangement of the channel receiving portions on the supporting unit. In some embodiments, the pattern is either regular (uniform) or irregular.

As used herein, the terms “array” and “needle array” refer to the regular or irregular spatial arrangements of the channel forming units positioned in desired relative positions, such as the positions predetermined or defined by the pattern on the supporting unit. In some embodiments, the array (first array, second array, and/or third array) are predetermined in different respective patterns (first pattern, second pattern, and/or third pattern) on the supporting unit. In some embodiments, the array is either regular or irregular.

As used herein, the term “holder unit” refers to a component of the device which is sized and shaped to receive and hold certain other components, for example, at least one supporting unit, at least a portion of the channel forming units, and provide at least certain remaining spaces for hydrogel portion. The holder unit and supporting unit together form a holder assembly.

As used herein, the terms “channel” and “microchannel” are used interchangeably when referring to space or cavity formed within the hydrogel portion. Such space or cavity may be introduced with certain elements, compositions, cells and/or compounds etc.

As used herein, the term “channel forming unit” refers to a component of the device that has a defined space to form a channel. In some embodiments, the channel forming unit generally is an elongated shaft. In some embodiments, a channel forming unit is flexible or malleable, which can be manipulated into different shapes and/or orientation. In some embodiments, a channel forming unit is in the form of a needle or wire.

As used herein, the term “hydrogel” refers to a phase transitional material containing crosslinked polymer chains and a fluid (such as water or PBS) that can form a gel-like structure. In some embodiments, in certain conditions, the hydrogel is prepared from a hydrogel composition and it is generally in the form of solid or semi-solid. Example hydrogel includes but not limited to proteins or gelling agents and optionally with cross-linking agents such as enzymes. In certain conditions (such as heat), the hydrogel in solid or semi-solid form may be removed, decomposed, dissolved, or melted. Examples of hydrogel include but not limited to collagen, alginate, gelatin, gellan gum, fibrinogen, xanthan gum, cellulose, plant protein (e.g. soy protein hydrogels, pea protein, or other vegetable protein), chitosan, carrageenan, starch hydrogels, agarose, pectin, guar gum, konjac glucomannan, lignin based hydrogels, and combinations thereof.

As used herein, the term “sacrificial layer” refers to a layer made of phase transitional material (liquid to solid or semi solid) such as ice and hydrogel that is formed and may be sacrificed (for example, removed, decomposed, melted or dissolved) in a later step.

As used herein, the term “hydrogel construct” refers to an engineered product formed by a hydrogel portion having a plurality of channels in a predetermined array and optionally (i.e., with or without) one or more cell compositions that are introduced into the channels within the hydrogel portion. In some examples, the cells in hydrogel construct are grown to form a tissue construct.

As used herein, the term “tissue construct” refers to an engineered product that mimics a natural cell tissue, and such tissue is formed from at least one cell composition. In some embodiments, a tissue construct is cultured-meat.

As used herein, the term “cell culture” refers to a population of cells that are grown under controlled conditions. In some examples, the cell culture is or includes a tissue culture in which the cells are grown and/or differentiated in the channels.

It is to be understood that terms such as “top”, “bottom”, “middle”, “side”, “length”, “inner”, “outer”, “interior”, “exterior”, “outside”, “vertical”, “horizontal”, “proximal”, “distal” and the like as may be used herein, merely describe points of reference and do not limit the present invention to any particular orientation or configuration. Further, terms such as “first”, “second”, “third”, “fourth”, etc., merely identify one of a number of portions, components and/or points of reference as disclosed herein, and likewise do not limit the present invention to any particular configuration, orientation or order. The first, second, third, fourth etc. portions, components and/or points of reference can be generally referred as the particular portions, components and/or points of reference. For example, the first cells, the second cells, the third cells and/or the fourth cells can be generally referred as “cells”.

As used herein and in the claims, the term “in fluid communication” refers to at least one fluid (such as a liquid), or at least one gas, or combination thereof, flowing through from one component to another, as circumstances indicate.

As used herein and in the claims, “connecting”, “engaging” means directly or indirectly physically bound to other elements.

### EMBODIMENTS OF THE PRESENT INVENTION

### Embodiment 1

In certain embodiments, provided is a method of producing cultured tissue. The method comprises the steps of (a) providing a hydrogel composition and solidifying the hydrogel composition to form a hydrogel portion comprising a plurality of first channels and a plurality of second channels, and (b) providing at least one first cell composition to the plurality of first channels to form at least one first cell culture, and providing with at least one second cell composition to the second channels to form at least one second cell culture, such that that a tissue construct is formed.

In certain embodiments, the cultured tissue is cultured meat, the first cell composition comprises a plurality of first cells that are muscle derived cells, muscle satellite cells and/or myoblast derived cells and the second cell composition comprises a plurality of second cells that are fat derived cells.

In certain embodiments, the hydrogel composition comprises one or more of alginate, gelatin, gellan gum and combination thereof.

In certain embodiments, the hydrogel composition comprises alginate at about 2% and gelatin at about 10%.

In certain embodiments, the hydrogel composition further comprises fat derived cells and fibroblast cells, and combination thereof.

In certain embodiments, the first cell composition comprises muscle derived cells, muscle satellite cells and/or myoblast derived cells, and optionally one or more of collagen, gellan gum, alginate, gelatin, and combination thereof.

In certain embodiments, the second cell composition comprises fat derived cells, and optionally one or more of collagen, gellan gum, alginate, gelatin, and combination thereof.

In certain embodiments, the method further comprises the step of: (c) providing a plurality of third channels and optionally providing at least one third cell composition therein.

In certain embodiments, the third cells comprise fibroblasts, endothelial cells, and combination thereof.

In certain embodiments, the at least one or more first cells, second cells and/or third cells are derived from fibroblasts, endothelial cells, myoblast cells, muscle cells, fat cells, skin cells, tendon, liver, brain, bone, heart, kidney, and combination thereof.

In certain embodiments, the plurality of the first, second and/or third channels are formed by removing a plurality of first, second and/or third channel forming units from a solidified hydrogel composition.

In certain embodiments, the plurality of the first, second and/or third channels are formed by 3-D printing or laser ablation.

In certain embodiments, the method further comprises the step of: (d) growing the first cells and the second cells until a desired tissue mass is obtained, thereby obtaining a cultured tissue product.

In certain embodiments, the step of (d) is performed by perfusing oxygenated medium through the tissue construct by the plurality of third channels.

In certain embodiments, the method further comprises the step of: (e) growing the third cells until a desired tissue mass is obtained.

In certain embodiments, the step (c) is performed after step (b), and wherein the plurality of third channels are formed by extruding out a plurality of perfusion channels by the plurality of third channel forming units.

In certain embodiments, the plurality of third channel forming units are biopsy punch tool.

In certain embodiments, the method further comprises the step of: treating the tissue construct with a hydrogel shrinking agent.

In certain embodiments, the method further comprises the step of: exercising the tissue construct electrically and/or mechanically.

In certain embodiments, prior to the step (b), the method comprises a step of coating the plurality of first channel forming units and/or the plurality of second channel forming units with lubricant (such as oil).

In certain embodiments, the hydrogel shrinking agent comprises low molecular weight (e.g., 15 kDa) chitosan.

In certain embodiments, the method further comprises the step of: crosslinking the hydrogel composition with 0.3 M calcium chloride solution.

In certain embodiments, the step of solidifying a hydrogel composition is performed by incubating the hydrogel composition at low temperatures (for example, about 4° C.).

In certain embodiments, the fat derived cells are derived from adipose derived stem cells from cow (such as Wagyu Ribeye).

In certain embodiments, the muscle derived cells are derived from myoblast derived stem cells from cow (such as Wagyu Ribeye).

In certain embodiments, provided is a device of producing cultured tissue. The device comprises a plurality of first channel forming units, each having a first diameter, a plurality of second channel forming units, each having a second diameter, and at least one support plate. The at least one support plate comprises a plurality of first channel receiving portions, each is sized and shaped to receive at least a portion of a first channel forming units and to define the first needle at a first position, and a plurality of second channel receiving portions, each is sized and shaped to receive at least a portion of a second channel forming units and to define the second needle at a second position. The plurality of first channel receiving portions and the plurality of second channel receiving portions are constructed and arranged in an array such that the plurality of first channel forming units and the plurality of second channel forming units can be assembled together by the at least one support plate to form a channel array and a plurality of spaces therebetween. The plurality of spaces are configured to receive a hydrogel composition to form a solidified, hydrogel portion, such that a plurality of first channels and a plurality of second channels are formed by removing the plurality of first channel forming units and the plurality of second channel forming units from the hydrogel portion, respectively. Each first channel is configured to receive at least one first cell composition therein to form at least one first cell culture, and each second channel is configured to receive at least one second cell composition therein to form at least one second cell culture, thereby a tissue construct is produced.

In certain embodiments, the device further comprises a plurality of third channel forming units to form a plurality of third channels for receiving at least one third cells therein.

In certain embodiments, the first diameter and the second diameter are larger (e.g., about 1-20 times larger) than diameters of target first cell culture and target second cell culture respectively.

In certain embodiments, the first cells composition comprises at least one first cell, and the second cell composition comprises at least one second cells.

In certain embodiments, the cultured tissue is cultured meat, the first cells are muscle derived cells, muscle satellite cells and/or myoblast derived cells and the second cells are fat derived cells.

In certain embodiments, the first diameter is about 0.1-10,000 μm, for example, about 20-500 μm.

In certain embodiments, the first diameter is less than 200 μm, for example, 20-90 μm.

In certain embodiments, a distance between two adjacent first channel forming units is about 0.1-5,000 μm, for example, 0.1-500 μm.

In certain embodiments, the distance between two adjacent first channel forming units is less than 100 μm, for example, 0.1-30 μm.

In certain embodiments, the second diameter is about 0.1-10,000 μm, for example, about 20-500 μm.

In certain embodiments, the second diameter is less than 500 μm, for example, 100-300 μm.

In certain embodiments, a distance between two adjacent second channel forming units is about 0.1-5,000 μm, for example, 0.1-500 μm, for example, about 1 μm.

In certain embodiments, the distance between two adjacent second channel forming units is less than 100 μm, for example, 0.1-30 μm.

In certain embodiments, the third cells comprise at least one or more of fibroblasts, endothelial cells, myoblast cells, muscle derived cells, fat derived cells, and combination thereof.

In certain embodiments, individual of the plurality of first and/or second channel forming units has a cylindrical, tubular structure.

In certain embodiments, individual of the plurality of first and/or second channel forming units has an open, trough structure with a U-shaped cross-section.

In certain embodiments, the tissue construct comprises a plurality of fat regions and a plurality of muscle regions.

In certain embodiments, provided is a system of producing cultured tissue, which comprises a device as described in any one of the preceding embodiments to produce a tissue construct having a plurality of third channels, a tissue construct adapter, configured to connect with the tissue construct, a bioreactor for oxygenating a medium, at least one pump system for circulating the medium from the bioreactor to the tissue construct through the plurality of third channels, and optionally a media recycler to remove waste.

In certain embodiments, the cultured tissue is cultured meat, the first cells are muscle derived cells, muscle satellite cells and/or myoblast derived cells and the second cells are fat derived cells.

In certain embodiments, the cultured tissues are derived from animals selected from the group consisting of mammals, birds, fish, invertebrates, reptiles, and amphibians.

In certain embodiments, the animals are human.

In certain embodiments, the animals are non-human.

In certain embodiments, the cultured tissue is cultured meat.

In some embodiments, the provided devices, systems and methods have one or more of the following features:
1. Forming microchannels (i.e., the plurality of first and/or second channels) in an edible hydrogel in whatever manner desirable (e.g., using channel forming units such as needles, biopsy punch tools, wires, 3D printed, molded, laser ablated).
2. Incorporating multiple cell types (muscle, fats, endothelial, fibroblast and/or other structural cells) in the microchannels. Cultured tissue does not have to be derived from skeletal muscle, so cells can also be derived from skin, tendon, liver, brain etc. In other words, other types of cultured tissues can be produced for various purposes such as but not limited to cultured meat, tissue engineering and organ transplantation.
3. Exercising the vascularized system becomes possible (which can help enhance cell growth, and texture).
4. Incorporating a perfusion system (oxygenated media, pumping through the channels) to mimic a vascularized system.
5. The microchannels that are perfused with oxygenated media has a channel-channel distance ranging from 0.5 μm-5 mm.
6. Shrinking the microchannels to make the dimensions smaller (with a shrinking agent).
7. Cell constructs formed by seeding cells in the microchannels (e.g. fat fibers, muscle fibers) and the diameters of the microchannels and the distance between any two adjacent microchannels are fully controlled. For example, the diameters and the distance is in the interquartile range of the desired meat tissue's tissue (e.g. the interquartile range of muscle fibers found in Wagyu meat).

In some embodiments, an array of 200 μm microchannels with 100 μm distance between two adjacent microchannels have been created with gelatin/alginate hydrogels. Muscle derived cells suspended in gelatin/transglutaminase hydrogels have been injected into the channels to grow.

In some embodiments, the space between two adjacent individual channels is less than 150 μm. In some embodiments, the space between two adjacent individual channels is less than 20 μm.

In some embodiments, the hydrogel composition comprises edible hydrogel.

In some embodiments, the hydrogel composition comprises natural hydrogels and/or non-natural hydrogels. In some embodiments, the hydrogel composition plant-based and/or non-plant-based hydrogels. In some embodiments, the hydrogel composition is naturally derived. In other embodiments, the hydrogel composition is synthetically made.

In some embodiments, cells (e.g., first cells, second cells and/or third cells) are derived from mammalian species. In some embodiments, the cells are derived from non-mammalian species.

In some embodiments, the cells are derived from eukaryotic cells. In some embodiments, the cells are derived from prokaryotic cells. In some embodiments, the cells are derived from microbes such as bacteria, fungi and virus.

In some embodiments, the integrity of the plurality of third channels is maintained during incubation and perfusion.

In certain embodiments, cultured tissues are derived from animals selected from the group consisting of mammals, birds, fish, invertebrates, reptiles, and amphibians.

In certain embodiments, the animals are non-human.

In some embodiments, the tissue construct is formed by seeding and incubating at least one first cell culture and at least one second cell culture.

In certain embodiments, the first diameter and the second diameter are about 1-20 times larger than diameters of target first cell culture and target second cell culture respectively. In certain embodiments, the first diameter and the second diameter are about 1, 2, 3, 4, 5, 6, 7, 8, 9, 10, 11, 12, 13, 14, 15, 16, 17, 18, 19 or 20 times (or even more) larger than diameters of target first cell culture and target second cell culture, respectively. In certain embodiments, the first diameter and the second diameter are about 3 times larger than diameters of target first cell culture and target second cell culture, respectively. In certain embodiments, the first diameter and the second diameter are about 1, 2, 3, 4, 5, 6, 7, 8, 9, 10, 11, 12, 13, 14, 15, 16, 17, 18, 19 or 20 times (or even more) larger than diameters of target first tissue and target second tissue, respectively. In certain embodiments, the first diameter and the second diameter are about 3 times larger than diameters of target first tissue and target second tissue, respectively.

For the sake of clarity, at least one (first or second) cell comprises one or more cells in one or more cell types or cells strains.

In certain embodiments, the first diameter is less than about 90 μm, for example, about 50 μm.

In certain embodiments, the second diameter is less than about 500 μm, for example, about 135 μm.

In certain embodiments, distance between two adjacent first channel forming units is less than about 15 μm, for example, about 5 μm.

In certain embodiments, distance between two adjacent second channel forming units is less than about 15 μm, for example, about 5 μm.

In some other embodiments, the first cells and/or second cells are derived from fibroblasts, endothelial cells, myoblast cells, muscle cells, fat cells, skin cells, tendon, liver, brain, bone, heart, kidney and combination thereof.

In some embodiments, the hydrogel composition comprises natural hydrogels and/or non-natural hydrogels. In some embodiments, the hydrogel composition plant-based and/or non-plant-based hydrogels. In some embodiments, the hydrogel composition is naturally derived. In other embodiments, the hydrogel composition is synthetically made.

In some other embodiments, the hydrogel composition further comprises cells derived from fibroblasts, endothelial cells, myoblast cells, muscle cells, fat cells, skin cells, tendon, liver, brain, bone, heart, kidney and combination thereof.

In certain embodiments, the method further comprises the step of: differentiating the first cells and/or the second cells into mature cell tissues.

In certain other embodiments, the step (e) is performed together with step (c), and wherein the plurality of third channels are formed by removing a plurality of third channel forming units from the hydrogel portion.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Designing a Desired Muscle Fiber/Fat Fiber Dimensions with Reference to a Selected Section of Natural Wagyu Meat Tissue

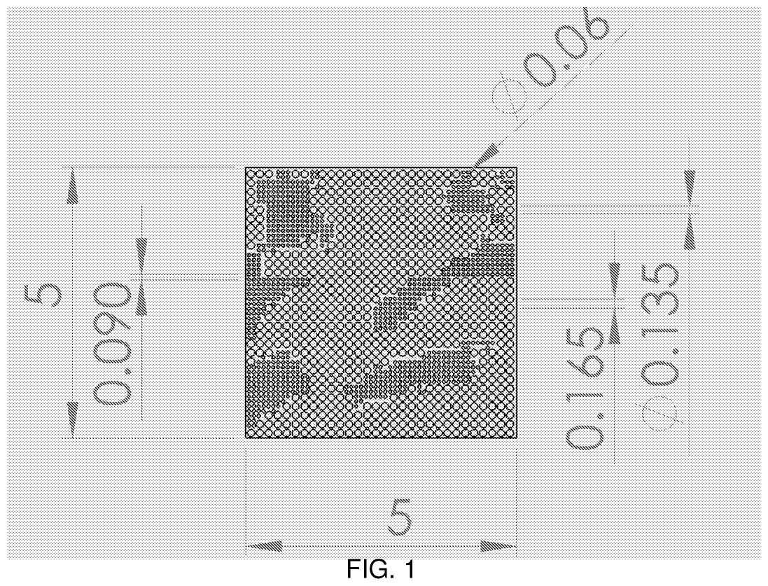

Now referring to FIG. 1, a cross-sectional view of an example design of a channel array (or microchannel array) with a desired/target muscle fiber/fat fiber dimensions (all figures are showing units in mm), mimicking the dimensions of a section of an in-vivo Wagyu tissue. The microchannel array has a total dimension of about 5×5 mm. The microchannel array comprises multiple muscle regions formed by a plurality of first channels and fat regions formed by a plurality of second channels. In this example, each of the plurality of first channels has a smaller first diameter of about 60 μm, and the distance between the two adjacent channels is about 30 μm. Each of the plurality of second channels has a larger second diameter of about 135 μm, and the distance between two adjacent second channels is about 30 μm.

### Devices and Systems for Producing Cultured Tissue

### Example 2: Channel Array

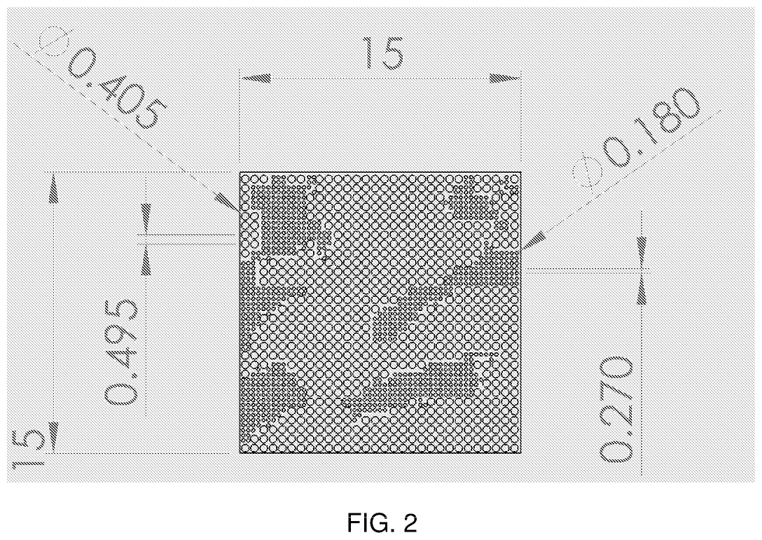

Now referring to FIG. 2, a cross-sectional view of another example design of channel array. In this example, based on the design described in Example 1, the channel array has a dimension of about three times larger than that example design in Example 1, i.e., about 15×15 mm. Each of the plurality of first channels has a first diameter of 180 μm, and the distance between two adjacent first channels is about 90 μm. Each of the plurality of second channels has a second diameter of about 405 μm and the distance between two adjacent second channels is about 90 μm. The channel array has an overall dimension (e.g., about 3 times) larger than the desired final dimensions of channel array to allow production of a cultured tissue in larger dimensions and shrinking in the subsequent steps.

### Example 3: Channel Assembly

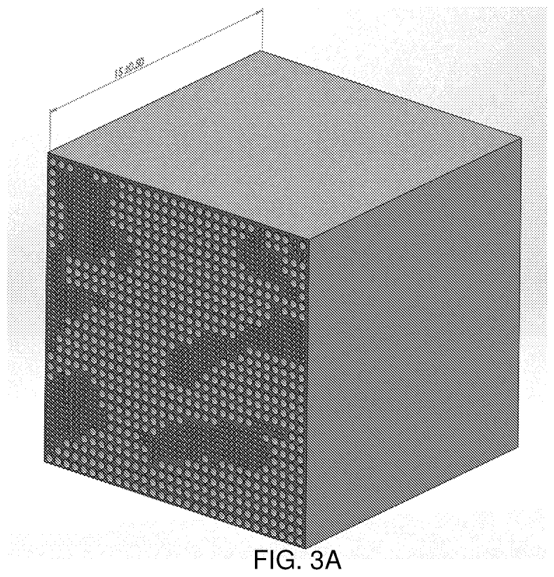

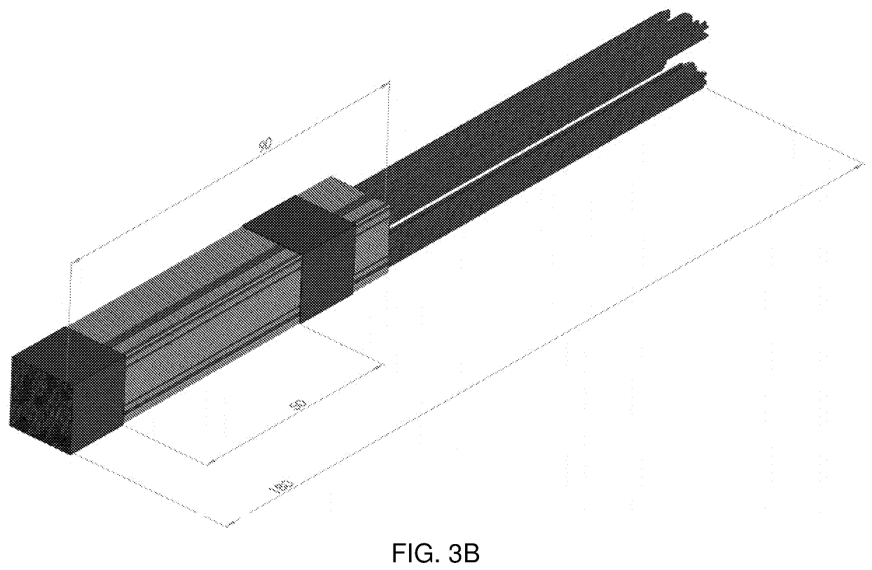

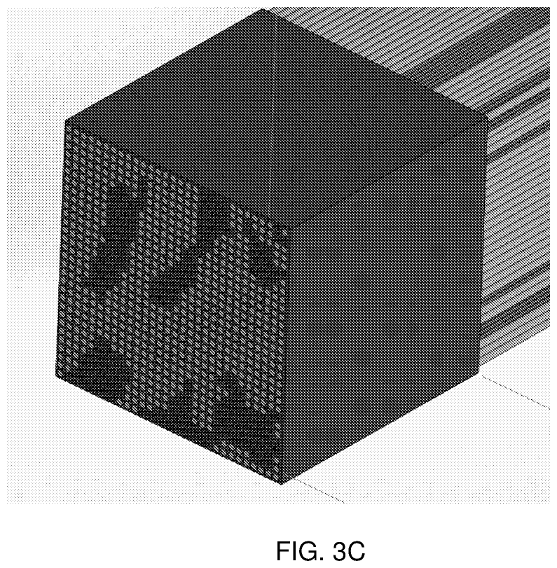

Now referring to FIGS. 3A-3C, showing an example channel assembly (i.e., two support plates together with the first and second channel forming units (in this example, first needles and second needles were used as examples of the first and second channel forming units, respectively) forming a needle array and a plurality of spaces therebetween). FIG. 3A shows a metal support plate comprising a plurality of first needle receiving portions (or holes) and a plurality of second needle receiving portions (or holes), the needle receiving portions are constructed and arranged based on the channel array as described in Example 2. Each needle receiving portions are sized and shaped to receive at least a portion of a needle and to define the need at its position. The diameter of each needle receiving portions is slightly larger than the channel diameter to receive at least a portion of a needle. Each first needle has a first diameter of a first channel (about 180 μm in this example), and each second needle has a second diameter of a second channel (about 405 μm in this example).

FIG. 3B shows an example channel assembly comprising two support plates with a plurality of first and second needles installed through the respective first and second needle receiving portions. The two support plates are shown in dark grey. First needles for muscle formation are shown in green and has a first needle length of about 180 mm. Second needles for fat formation are shown in light grey and has a second needle length of about 90 mm.

FIG. 3C is a zoomed-in view of the channel assembly, showing that the first needles (in green) are in the first needle receiving portions for muscle formation later and the second needles (in grey) are inserted into in the second needle receiving portions of the support plate for fat formation later.

### Example 4: Tissue Construct

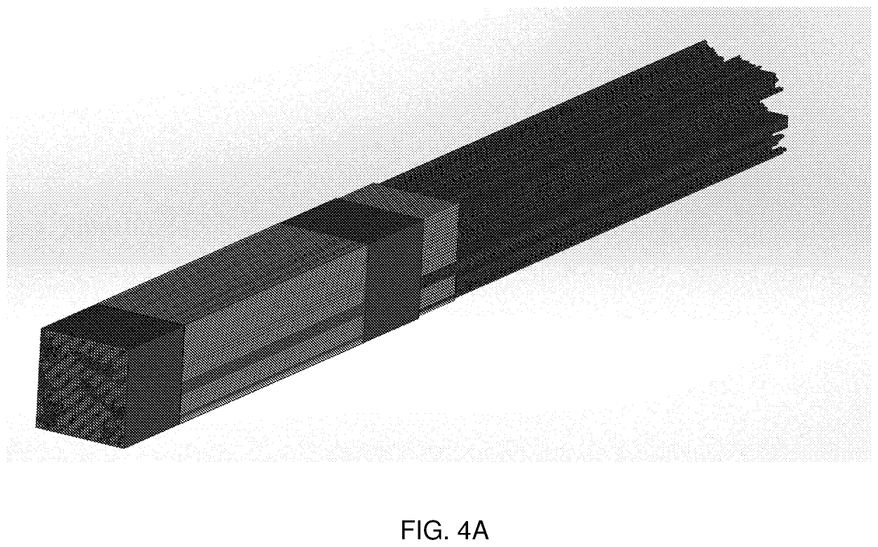

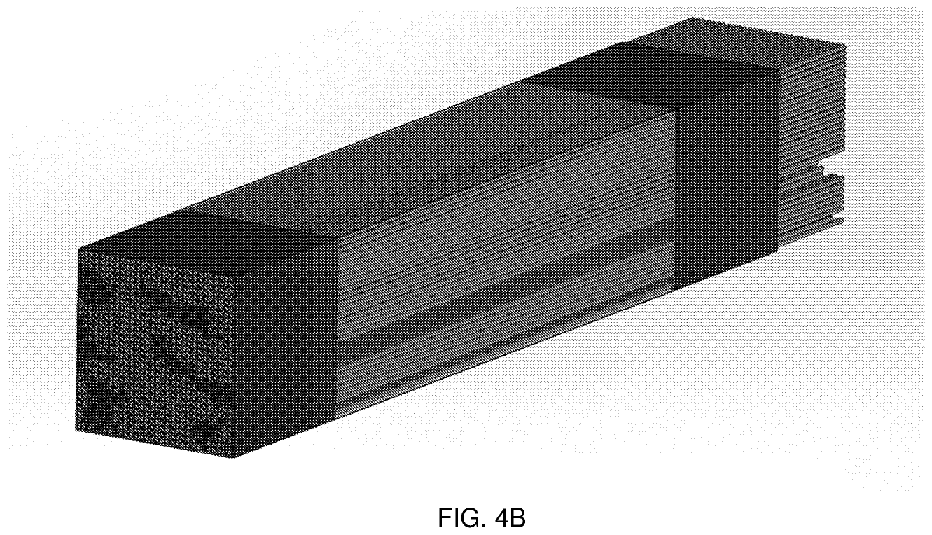

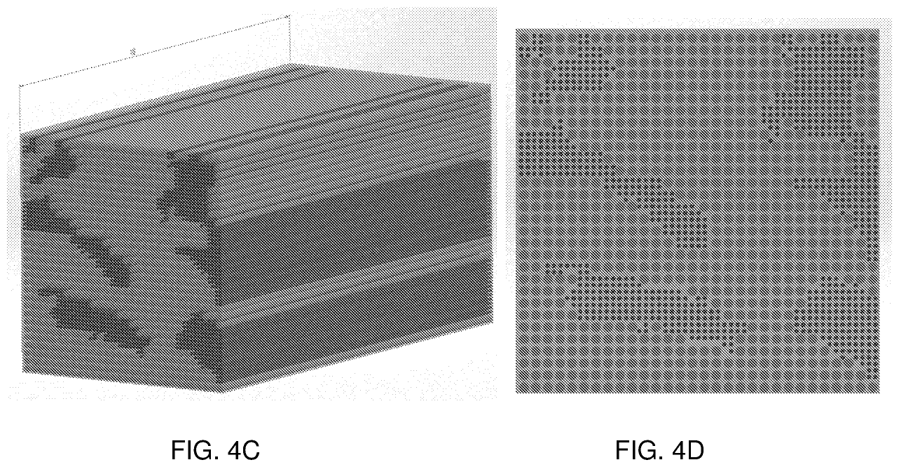

Now referring to FIGS. 4A-C, showing the formation of a tissue construct (comprising hydrogel portion and channel portion). FIG. 4A is an example channel assembly (as described in Example 3), filled with a hydrogel composition (Mix A) to form a hydrogel portion. Hydrogel composition was poured over the plurality of spaces among the needle array of the channel assembly to form the hydrogel portion shown in red. In this example, Mix A comprises about 2% sodium alginate and about 10% gelatin was used. In other examples, Mix A comprises about 2% alginate and 20% gelatin. In other examples, 0.1-10% (such as 0.5%) alginate can be used instead. Mix A was allowed to solidify under low temperatures (e.g., 4° C.). Mix A was crosslinked with 0.3 M calcium chloride solution to form a hydrogel portion. In other examples, less than 1% calcium chloride solution was used for crosslinking. In other examples, less than 1% calcium carbonate solution was used for crosslinking.

FIG. 4B shows the example channel assembly having the hydrogel portion with a plurality of first channel forming units (in this example, the first channel forming units were first needles, such as the green needles shown in FIG. 4A) removed from the channel assembly. The plurality of first needles was removed from the hydrogel portion, forming a plurality of first channels where muscle cells could be seeded into. In this example, a first cell composition (Mix B) comprising myoblasts cells (1×10⁷ cells/ml) derived from Wagyu ribeye suspended in Type 1 collagen (TeloCol®-6 Type I Collagen Solution, 6 mg/ml (Bovine)) was used for seeding into the first channels. The first cell composition were seeded first then forming the first channels together with the first cell composition therein by removing the first needles from the hydrogel portion. A neutralization solution (10 ml, TeloCol®-6 Type I Collagen Solution, 6 mg/ml (bovine), Advanced BioMatrix) was also added in Mix B (about 1/10th of the final volume was used) for crosslinking to occur under about 37° C.

The plurality of second channel forming units (in this example, the second channel forming units were second needles) was removed from the hydrogel portion without a particular order, i.e., separately, before or after the step of removing the first needles, or simultaneously. In this example, the plurality of second needles was removed from the hydrogel portion subsequently to the step of obtaining the cultured first cell culture forming a plurality of second channels where fat derived cells could be seeded into. Second cell composition (Mix C) comprising fat derived cells (1×10⁷ cells/ml) suspended in Type 1 collagen (TeloCol®-6 Type I Collagen Solution, 6 mg/ml (Bovine)) were seeded into the remaining second channels. In this example, adipose derived stem cells from Wagyu Ribeye were used. A neutralization solution was also added in Mix C (about 1/10th of the final volume was used) for crosslinking to occur under about 37° C.

FIGS. 4C and 4D show an example tissue construct comprising hydrogel portion and channel portion seeded with both first cell composition (comprising fat derived cells) and second cell composition (comprising muscle derived cells), after removing the needles and support plates and seeded with fat derived cells and muscle derived cells.

### Example 5: Tissue Construct with Perfusion Channels

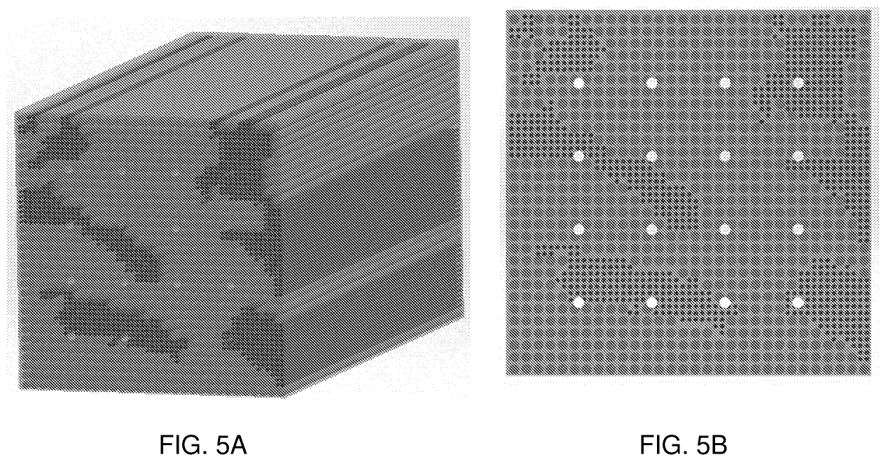

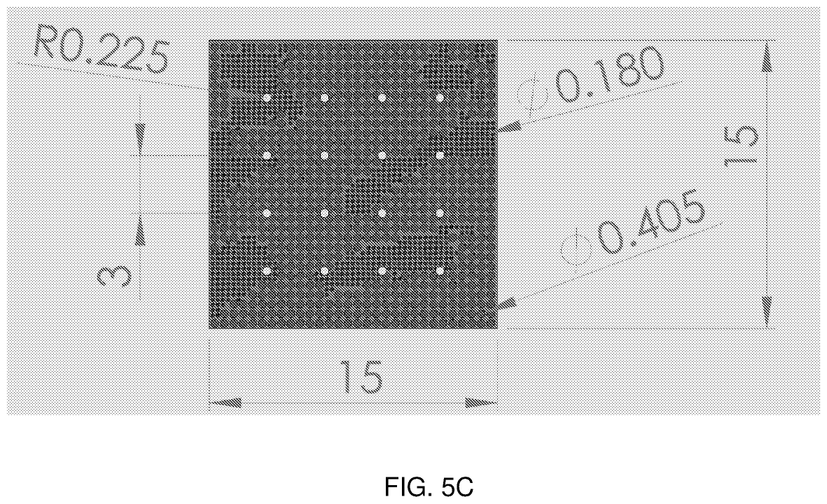

Now referring to FIGS. 5A-5C. FIGS. 5A and 5B show an example tissue construct formed in FIG. 4C but subsequently being extruded out with a plurality of perfusion channels (third channels) by biopsy punch tools. FIG. 5B is a cross-sectional view of the example tissue construct perfused with a plurality of third channels. In this example, the perfusion channels formed a 4×4 perfusion array. A biopsy punch tool was used to extrude out the perfusion channels. In some embodiments, endothelial cells can be seeded in the third channels to form endothelial tubes along the perfusion channels.

In other examples, the perfusion channels can be created by providing a plurality of third channel forming units (such as needles) in the channel assembly (such as described in Example 2) first, pouring third cell composition (Mix D, comprising endothelial cells) and let the third cells to grow. In some embodiments, the third cells comprise endothelial cells, which will grow and differentiate along the surface of the third channels. The third channels are configured so as to fluidly communicate with a perfusion assembly system.

FIG. 5C shows the dimensions of the example tissue construct of FIGS. 5A and 5B. In this example, the diameter of each of the third channels is about 225 μm and the distance between two adjacent third channels) is about 3 mm.

### Example 6: Tissue Construct Adapter and Perfusion Assembly

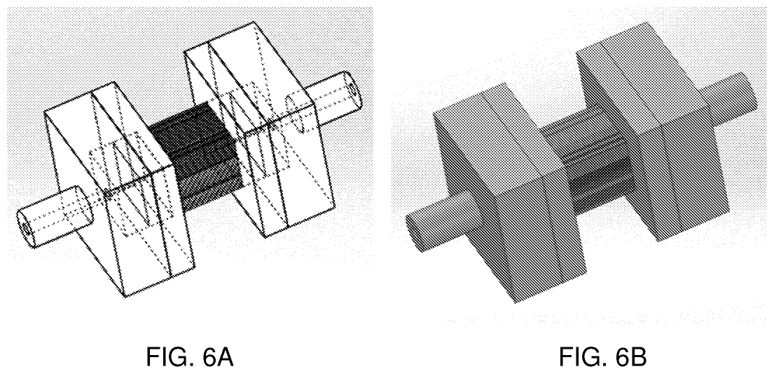

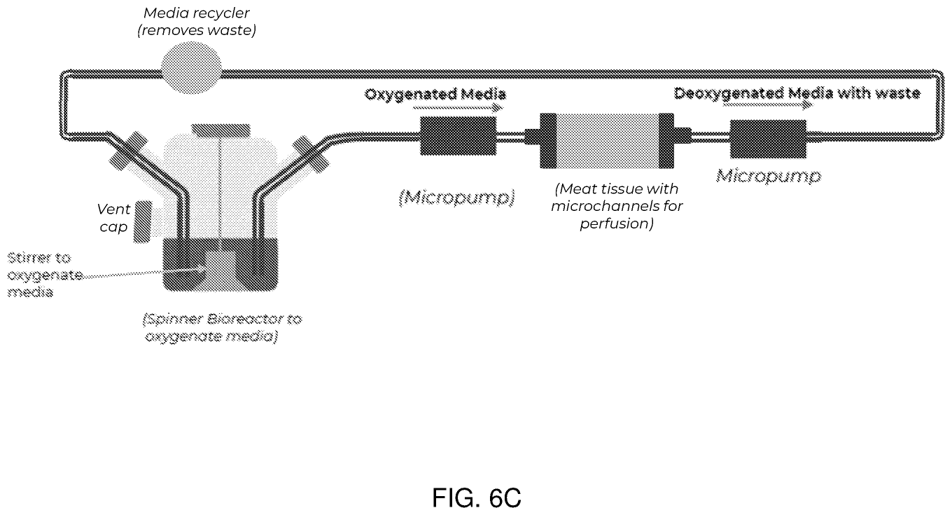

FIGS. 6A and 6B shows a tissue construct sandwiched between a tissue construct adapter.

FIG. 6C shows a perfusion assembly system which contains pumps, bioreactor, oxygenated media, tissue construct (meat) and tissue construct adapter. The tissue construct adapter (or called tube adapter) is attached to the meat tissue construct (such as described in Example 5) to form a tissue construct assembly and a tubing is connected with the tissue construct assembly and an oxygenated media source. In this example, the pumps were a first micropump and a second micropump located upstream and downstream of the tissue construct assembly, respectively, the bioreactor was a spinner bioreactor with a stirrer to oxygenate media and the tissue construct assembly had a plurality of microchannels (third channels for perfusion) connected with the perfusion assembly by the tubing. In this example, the oxygenated media source came from a 100 mL bioreactor with a PTFE filter to allow for sterile air to come in and be in contact with the media. When the media was mixed, air (and oxygen) was dissolved to oxygenate the media. One or more pumps were used to pump the oxygenated media into the tissue construct, and deoxygenated media with waste from the tissue construct. As such, the tissue construct is perfused with the media (by perfusion step). Optionally a media recycler was connected downstream of the tissue construct to remove any waste. The tissue construct was then allowed to mature.

### Example 7: Final Product of Tissue Construct

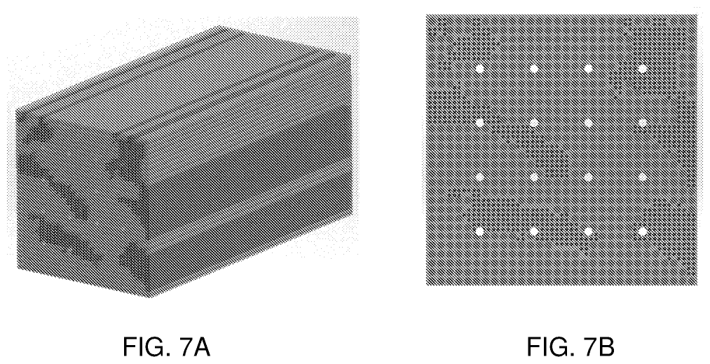

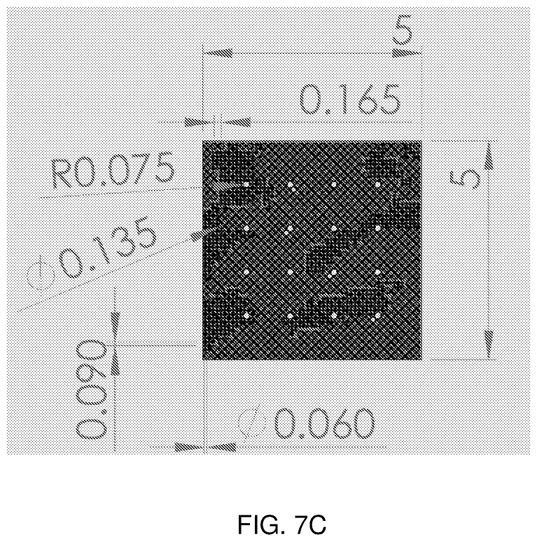

Now referring to FIGS. 7A-7C. FIGS. 7A and 7B show a final target product of meat tissue construct from Example 6 subsequently undergoing a shrinking step. FIG. 7B shows a cross-sectional view of the final meat tissue product mimicking Wagyu meat tissue. The tissue construct was treated with a shrinking agent to shrink the tissue. In this example, low molecular weight chitosan (e.g., about 15 kDa) solution was used so the tissue construct was shrunk by about 3-fold. As such, the tissue can be exercised to induce greater matrix re-arrangement and thus, texture. In other examples, the tissue construct can be immersed in a solution such as acetic acid (i.e., vinegar) that can shrink the fiber-fiber distance such as to about 20 μm. In other examples, the forces exerted by muscle fibers will cause (such as about 1-20 times, say, about 3 times) shrinkage (may vary depending on the hydrogel used) in diameter to be more similar to a physiologically relevant diameter (50-66.7 μm muscle fiber diameters found in beef). In other examples, the shrinking step can be performed prior to the perfusion step as described in Example 6.

### Example 8: Methods of Producing Cultured Tissue

In this embodiment, cultured meat is used as an example of cultured tissue and the example method of producing cultured meat comprises steps of preparing a channel assembly followed by preparing a tissue construct.

In some embodiments, a channel assembly is prepared by the following steps:
(1) providing the devices or systems as described in the previous examples; oil coating the one or more support plates and channel forming units (such as needles), providing at least a plurality of first needles and a plurality of second needles with the support plates to form a channel assembly;
(2) rinsing the support plates; the plurality of first needles and the plurality of second needles were rinsed in water twice, wherein each wash cycle is 15 min, and sterilizing the support plates and the plurality of first and second needles (such as under UV light);
(3) assembling the support plates, the plurality of first needles and the plurality of second needles into a channel assembly. In one example, the diameter of each of the plurality of first needles was about 180 μm, with a distance between two adjacent first needles of 50-60 μm, and the diameter of each of the plurality of second needles was about 405 μm, with a distance between two adjacent second needles of 50-60 μm. In other examples, other diameters and distances as described in the previous examples may be used.
(4) pouring Mix A over the channel assembly, wherein the Mix A comprises one or more of cells (e.g., one or more of muscle derived cells, fat derived cells, fibroblasts and combination thereof), sodium alginate, gelatin, gellan gum, and combination thereof.
(5) allowing the Mix A to solidify under low temperature (e.g., 4° C.), so that the hydrogel portion is formed.

In some embodiments, a plurality of third needles is also provided and assembled with the channel assembly in the initial step (1), so that the channel assembly comprising a plurality of first, second and third needles is formed.

In some embodiments, the first tissue of a tissue construct is prepared by the following steps:
(1) pouring Mix B into a 6 well, wherein the Mix B comprises first cells (in this example, muscle or myoblast cells), collagen, sodium alginate, gelatin, gellan gum, crosslinking agent (e.g., calcium chloride and/or calcium carbonate solution), shrinking agent, microbial transglutaminase, culture medium (e.g., DMEM, FBS), neutralization solution, and combination thereof, or as described in the previous examples;
(2) removing the plurality of first needles away from the channel assembly until the first needles are almost nearing the tip of the channel assembly, forming a plurality of first channels. In Some examples, the needles should still be sticking out of the first channel section slightly;
(3) seeding Mix B as described in previous examples into the plurality of smaller channels slowly without shearing the cells nor introducing bubbles;
(4) ensuring the first cells are not flowing out of the first channels and allowing Mix B to solidify; and
(5) crosslinking the Mix B in the first channels at a temperature below 37° C. (e.g., about 4° C.).

In some embodiments, the method further comprises the step of: growing the first cells until a desired tissue mass is obtained, thereby obtaining a cultured first tissue (i.e., muscle tissue). In some embodiments, the growing step is performed as described in Example 6.

In some embodiments, prior to seeding in the first cells and removing first needles, simultaneously or subsequently thereto, the second cell culture of the tissue construct is prepared by the following steps:
(1) pouring Mix C into a 6 well, wherein the Mix C comprises second cells (in this example, fat derived cells), collagen, sodium alginate, gelatin, gellan gum, crosslinking agent (e.g., calcium chloride and/or calcium carbonate solution), shrinking agent, microbial transglutaminase, culture medium (e.g., DMEM, FBS), neutralization solution, and combination thereof, or as described in the previous examples;
(2) removing the plurality of second needles away from the channel assembly until the second needles are almost nearing the tip of the assembly, forming a plurality of second channels. In Some examples, the second needles should still be sticking out of the second channel section slightly;
(3) seeding Mix C as described in previous examples into the plurality of second channels slowly without shearing the cells nor introducing bubbles;
(4) ensuring the second cells are not flowing out the second channels and allowing it to solidify; and
(5) crosslinking the Mix C in the second channels at a temperature below 37° C. (e.g., about 4° C.).

In some embodiments, the method further comprises the step of: growing the second cells until a desired tissue mass is obtained, thereby obtaining a cultured second tissue (i.e., fat tissue). In some embodiments, the growing step is performed as described in Example 6.

In some embodiments, prior to the step of obtaining the cultured first and/or second tissues, simultaneously or subsequently thereto, the third tissue of the tissue construct, if present, is prepared by the following steps:
(1) pouring Mix D into a 6 well, wherein the Mix D comprises third cells (in this example, endothelial cells). Optionally, the Mix D further comprises collagen, sodium alginate, gelatin, gellan gum, crosslinking agent (e.g., calcium chloride and/or calcium carbonate solution), shrinking agent, microbial transglutaminase, culture medium (e.g., DMEM, FBS), neutralization solution, and combination thereof, or as described in the previous examples;
(2) seeding third cells and removing the plurality of third needles away from the channel assembly until the third needles are almost nearing the tip of the assembly, forming a plurality of third channels. In Some examples, the third needles should still be sticking out of the third channel section slightly. In some other examples, the third channels are formed by extruding out a plurality of perfusion channels as described in the previous examples;
(3) seeding Mix D as described in previous examples into the plurality of third channels slowly without shearing the cells nor introducing bubbles; and
(4) optionally ensuring the third cells are not flowing out the third channels and allowing the third cells to attach to the third channels such as to form endothelial tissue along the surface of the third channels. and
(5) optionally crosslinking the Mix D in the third channels. For example. crosslinking at a low temperature (for example, below 37° C. such as about 4° C.).

In some other embodiments, a plurality of third needles are formed by perfusing third channels after the tissue construct (or solidified hydrogel portion) is formed.

In some embodiments, the method further comprises the step of: growing the third cells until a desired tissue mass is obtained, thereby obtaining a cultured third tissue (i.e., endothelial tissue along the third channels). In some embodiments, the growing step is performed as described in Example 6.

In some embodiments, further comprising the step of: treating the tissue construct with a shrinking agent to shrink the tissue construct. In some examples, the tissue construct is shrunk to around 3 times smaller than the original size. In some embodiments, the shrinking agent is or comprises low molecular weight (such as 15 kDa) chitosan solution. In some embodiments, the shrinking step is performed as described in Example 7.

### Example 9a: Example Methods of Producing Cultured Tissue

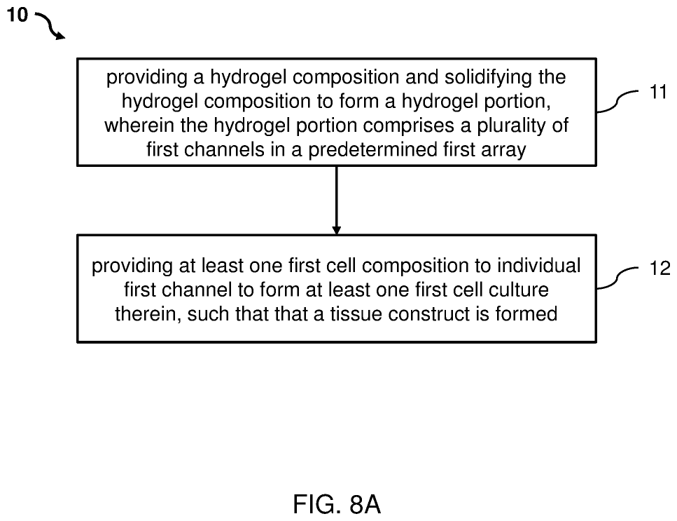

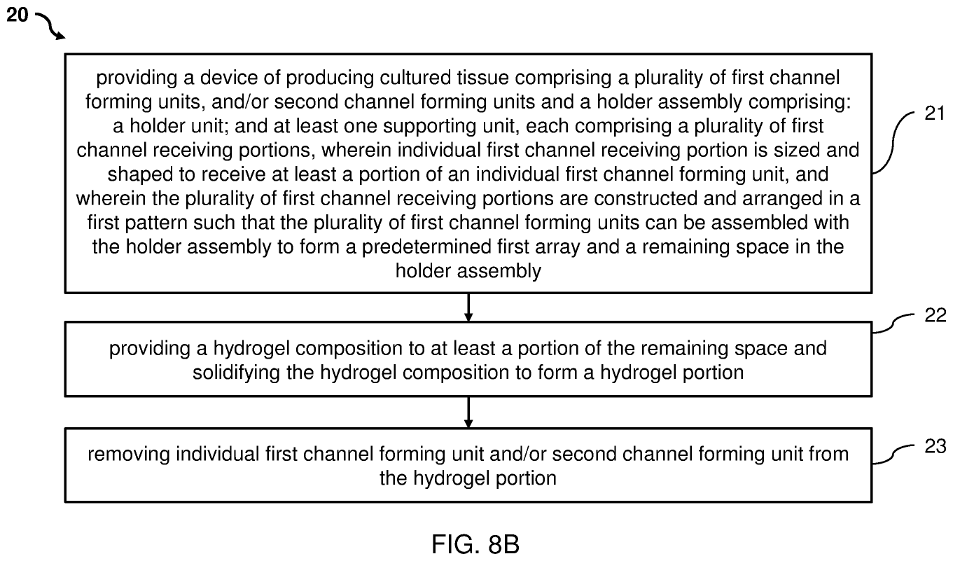

Now referring to FIGS. 8A-8B, flow charts showing example methods of producing cultured tissues. As shown in FIG. 8A, the method 10 of producing cultured tissues includes the following steps.

In step 11, providing a hydrogel composition and solidifying the hydrogel composition to form a hydrogel portion, wherein the hydrogel portion comprises a plurality of first channels in a predetermined first array is performed. In one implementation, the plurality of first channels are formed by providing a plurality of first channel forming units in the hydrogel composition before solidifying the same.

In one implementation, in step 11, the hydrogel portion further comprises a plurality of second channels in a predetermined second array. In one further implementation, the plurality of second channels are formed by providing a plurality of second channel forming units in the hydrogel composition before solidifying the same. In another implementation, the plurality of first and/or second channels are formed after solidifying the same. In one example, the first and second channel forming units (and channels) are provided simultaneously or sequentially in any order.

In one implementation, in step 11, the hydrogel portion further comprises a plurality of third channels in a predetermined third array. In one further implementation, the plurality of third channels are formed by providing a plurality of third channel forming units in the hydrogel composition before solidifying the same. In one example, the first, second and third channel forming units (and channels) are provided simultaneously or sequentially in any order. In another implementation, the plurality of third channels are formed by providing a plurality of third channel forming units in the hydrogel composition after solidifying the same (for example, after step 12). By way of example, the plurality of third channel forming units are biopsy punching tools and the plurality of third channels are formed after the hydrogel portion having the first and/or second channels is formed.

In step 12, providing at least one first cell composition to individual first channel to form at least one first cell culture therein is performed, such that that a tissue construct is formed. By way of example, one or more different first cell compositions are provided into individual or all of the first channels. The first cell compositions can be introduced simultaneously or sequentially in any order. In one implementation, the first cell compositions comprise a plurality of first cells that are muscle derived cells, muscle satellite cells and/or myoblast derived cells, and optionally further comprise and optionally one or more of collagen, alginate, gelatin, gellan gum, fibrinogen, and combination thereof. In one implementation, the second cell compositions optionally further comprise hydrogel, culture media and other ingredients. Example first cell compositions will be further described in other examples.

In one implementation, step 12 further comprising the step of: providing at least one second cell composition to individual second channel (if present) to form at least one second cell culture therein. By way of example, one or more different second cell compositions are provided into individual or all of the second channels. The second cell compositions can be introduced simultaneously or sequentially in any order. In one implementation, the second cell compositions comprise a plurality of second cells that are fat derived cells and optionally one or more of collagen, alginate, gelatin, gellan gum, fibrinogen, and combination thereof. In one implementation, the second cell compositions optionally further comprise hydrogel, culture media and other ingredients. Example second cell compositions will be further described in other examples.

In one implementation, step 12 further comprising the step of: providing at least one third cell composition to individual third channel (if present) to form at least one third cell culture therein. By way of example, one or more different third cell compositions are provided into individual or all of the third channels. The third cell compositions can be introduced simultaneously or sequentially in any order. In one implementation, the third cell compositions comprise a plurality of third cells that are fibroblasts, endothelial cells, and combination thereof and optionally one or more of collagen, alginate, gelatin, gellan gum, fibrinogen, and combination thereof. In one implementation, the third cell compositions optionally further comprise hydrogel, culture media and other ingredients. Example third cell compositions will be further described in other examples.

In one implementation, step 12 further comprising the step of: growing the plurality of first cells, the plurality of second cells and/or the plurality of third cells, if present, until a desired tissue mass is obtained. The growing step is performed by incubating or culturing the first, second and/or third cells under desired conditions. In some embodiments, the growing step enables differentiation and/or growth of the cells.

In some embodiments, a tissue construct was prepared using a device as described in any one of the examples described herein. The method 20 further includes one or more of the following steps:

In step 21, providing a device of producing cultured tissue comprising a plurality of first channel forming units, and/or second channel forming units and a holder assembly comprising: a holder unit; and at least one supporting unit, each comprising a plurality of first channel receiving portions, wherein individual first channel receiving portion is sized and shaped to receive at least a portion of an individual first channel forming unit, and wherein the plurality of first channel receiving portions are constructed and arranged in a first pattern such that the plurality of first channel forming units can be assembled with the holder assembly to form a predetermined first array and a remaining space in the holder assembly is performed.

In step 22, providing a hydrogel composition to at least a portion of the remaining space and solidifying the hydrogel composition to form a hydrogel portion is performed.

In step 23, removing individual first channel forming unit and/or second channel forming unit from the hydrogel portion is performed. In some embodiments, the first channel forming unit and/or the second channel forming unit were removed after the hydrogel composition is solidified.

Optionally, the assembled device further comprising a plurality of third channel forming units, individual third channel forming units are arranged in a predetermined third array. The third channel forming units are provided either before or after the hydrogel portion is formed.

For clarity sake, the above mentioned method steps can be performed sequentially, simultaneously, or in any other order if applicable.

### Example 9b: Preparation of Example Hydrogel Compositions and Example Cell Compositions

Example hydrogel compositions and cell compositions are listed in Table 1 and Table 2, respectively. In general, a hydrogel composition contains one or more proteins or gelling agents (e.g., gelatin, fibrinogen, alginate and/or gellan gum or the like) and optionally one or more corresponding cross-linking agents (enzymes or the like). Unless otherwise indicated, the hydrogel compositions and cell compositions were dissolved in phosphate buffered saline (PBS). These examples are presented for illustrative purposes only and are not intended to be an exhaustive list of all possible embodiments of the invention.

TABLE 1
Example hydrogel compositions

| | | Additional | |
|---|---|---|---|
| Hydrogel | | agent for | |
| composition | | Cross-linking | Cross-linking |
| name | Hydrogel Ingredients | (if any) | conditions |
| Mix A 1 | 2% sodium alginate and 20% | 0.3M calcium | 4° C. |
| | gelatin | chloride solution | |
| Mix A 2 | 2% sodium alginate and about | 0.3M calcium | 4° C. |
| | 10% gelatin | chloride solution | |
| Mix A 3 | 0.5% alginate | 0.3M calcium | 4° C. |
| | | chloride solution | |
| Formulation | 13.5% gelatin and 1% | n/a | r.t.; within |
| #1 | transglutaminase TI | | 48 hours |
| Formulation | 20% (w/v) gelatin and 2% (w/v) | 1% calcium | 4° C. |
| #2 | gellan gum | chloride | |
| Formulation | 20% (w/v) gelatin and 2% (w/v) | 1% calcium | 4° C. |
| #3 | sodium alginate | chloride | |
| Formulation | 5% gelatin dissolved in PBS and | n/a | 37° C.; within |
| #4 | 10 U/g transglutaminase TI | | 3 hours |
| Formulation | 20% (w/v) gelatin | n/a | r.t. |
| #5 | | | |
| Formulation | 1 mg/mL fibrinogen and 1 U/mL | n/a | 37° C. |
| #6 | thrombin | | |
| Formulation | 10% gelatin prepared in PBS | n/a | r.t. |
| #7 | | | |

Note:
r.t. = room temperature;
U = enzyme unit;
U/g = enzyme unit per grams of gelling agent

Methods of forming or preparing hydrogel portion using the example hydrogel compositions are described below.

### Preparation of Formulation #1:

To prepare the hydrogel composition Formulation #1 as described in Table 1, the following steps were carried out:
(1) 15% (w/v) gelatin (Sigma) solution was prepared by dissolving 15 g gelatin in 100 mL PBS. The 15% gelatin solution was heat up to 50° C. and autoclaved.
(2) 10% (w/v) transglutaminase TI solution in PBS was prepared by dissolving 10 g transglutaminase powder (resulting in about 100 to 120 enzyme units (U) of transglutaminase TI per gram of powder) in 100 mL PBS (10% Tg=0.1 g/mL=10 U/mL to 12 U/mL, where U denotes enzyme units). The 10% transglutaminase solution was sterile filtered through a 0.22 μm filter.
(3) The hydrogel composition of Formulation #1 (about 13.5% gelatin+about 1% transglutaminase (1 U/mL)) was prepared by mixing 9 parts 15% gelatin solution to 1 part 10% transglutaminase solution from (1) and (2). In some embodiments, 1 part 10% transglutaminase was first added to a centrifuge tube, followed by the addition of 9 parts 15% gelatin solution, and mixed immediately for 30 s before use. In this example, the final concentration of 1% transglutaminase contained about 7.4 to 8.88 U transglutaminase per gram of gelatin.

To prepare a hydrogel portion using the hydrogel composition Formulation #1, the hydrogel composition was transferred to a desired device or container allowed to crosslink in room temperature for 48 hours. In some embodiments, Formulation #1 was further incubated in 37° C. for 2 hours.

### Preparation of Formulation #4:

Procedures similar to the preparation of Formulation #1 described above were used to prepare hydrogel compositions with various concentrations of gelatin (or other gelling agents), and the concentration of transglutaminase TI was controlled within the range of 0.1-100 U transglutaminase per gram of gelatin (or other suitable gelling agents) used. In this example, to prepare Formulation #4, which contains 5% gelatin and 10 U/g transglutaminase TI (10 U transglutaminase per gram of gelatin), 10 U/g*0.05 g=0.5 U of transglutaminase TI was needed. If a 10% transglutaminase stock solution above is used, the volume needed would be 0.5 U/(10 U/mL)=0.05 mL or 0.5 U/(12 U/mL)=0.0416 mL. Formulation #4 was allowed to crosslink in 37° C. for 2-3 hours. In some embodiments, when Formulation #4 was used in a cell composition, the mixture was allowed to crosslink in 37° C. for 2-3 hours with occasional mixing via pipetting up and down.

### Preparation of Formulation #2:

20% (w/v) gelatin (Sigma)+2% (w/v) gellan gum was prepared by using PBS as the solvent, followed by autoclaving afterwards to obtain Formulation #2.

To prepare a hydrogel portion using the hydrogel composition Formulation #2, the hydrogel composition was first melted in boiling water. To prevent air bubbles from being trapped, the solution was occasionally transferred into the biosafety cabinet, and the cap was opened to release the pressure and bubbles. If air bubbles persisted, the solution was allowed to solidify in 4° C., and then melting was repeated. Once the hydrogel composition was sufficiently melted, it was transferred to a desired device or container. For crosslinking, the hydrogel composition Formulation #2 was immersed in 1% calcium chloride. In some embodiments, other crosslinking agents such as other calcium salts (such as calcium chloride), zinc salts (such as zinc chloride), magnesium salts, barium salts, sodium tripolyphosphate, or DMEM+10% FBS.

### Preparation of Formulation #3:

A 2% sodium aginate solution was first prepared using PBS as the solvent, and heated to 50° C. While hot, the 2% sodium alginate solution was sterile filtered through a 0.45 μm filter. Sterile gelatin powder (8 g) into was added to 40 mL of the 2% sodium alginate solution, and the mixture was autoclaved afterwards to obtain a hydrogel composition Formulation #3 with final concentrations of 20% (w/v) gelatin (Sigma)+2% (w/v) sodium alginate.

To prepare a hydrogel portion using the hydrogel composition Formulation #3, the hydrogel composition was first melted in a 37° C. bath. Once the hydrogel composition was sufficiently melted, it was transferred to a desired device or container. For crosslinking, the hydrogel composition Formulation #3 was immersed in 1% calcium chloride. In some embodiments, other crosslinking agents such as other calcium salts (such as calcium chloride), zinc salts (such as zinc chloride), magnesium salts, or barium salts.

### Preparation of Formulation #6:

Stock solutions of 20 mg/mL fibrinogen (in PBS) and 20 U/mL thrombin (in water containing 0.1% (w/v) Bovine Serum Albumin) were prepared. A hydrogel composition Formulation #6 having final concentrations of 1 mg/mL fibrinogen+1 U/mL of thrombin was prepared by mixing 5 μL of 20 mg/mL fibrinogen, 5 μL of 20 U/mL thrombin and 90 μL of PBS. The hydrogel composition was used immediately after mixing, as the crosslinking occurred in approximately 1 minute.

Other example hydrogel compositions are prepared using similar methods as described above with suitable adjustments.

Example cell compositions are shown in Table 2. These examples are presented for illustrative purposes only and are not intended to be an exhaustive list of all possible embodiments of the invention.

TABLE 2
Example cell compositions

| Cell | |
|---|---|
| composition | |
| name | Ingredients |
| B | 1 × 10⁷ cells/ml of myoblasts cells derived from Angus cow suspended |
| | in Type 1 collagen (TeloCol ®-6 Type I Collagen Solution, 6 mg/ml |
| | (Bovine)) |
| C | 1 × 10⁷ cells/ml of fat derived cells derived from Angus cow suspended |
| | in Type 1 collagen (TeloCol ®-6 Type I Collagen Solution, 6 mg/ml |
| | (Bovine)) |
| D | 5 × 10⁶ cells/ml of endothelial cells suspended in DMEM + 10% FBS |
| | (for perfusion assembly system) |
| E | 1 × 10⁵ cells/mL of porcine primary myoblast cells suspended in PBS |
| | or sterile water |
| F | 1 × 10⁵ cells/mL of porcine primary myoblast cells suspended in |
| | Hydrogel Formulation #6 |
| G | 1 × 10⁶ cells/mL of porcine myoblasts suspended in PBS or sterile |
| | water |
| H | 1 × 10⁶ cells/mL of porcine myoblasts suspended in Hydrogel |
| | Formulation #6 |
| I | 1 × 10⁶ cells/mL of porcine myoblasts suspended in Hydrogel |
| | Formulation #4 |
| J | 1 × 10⁷ cells/mL of porcine myoblast cells suspended in Hydrogel |
| | Formulation #6 |
| K | Formulation #6 + 1 × 10⁵ cells/mL (porcine mature adipocytes) |

Cell compositions were generally prepared by rinsing the cells twice with 0.1 mL per cm² sterile PBS (passage 6 or less) and harvesting the cells from one or more of the passages. Cells from passage 5 were used. The trypsin volume and trypsin neutralizer solution volume used was 0.053 mL per cm². For example, 4 mL of 0.25% trypsin/EDTA was added to the cells in a T75 flask (0.053 mL/cm^{2*75} cm²=about 4 mL) and incubation for about 5 minutes at about 37° C. After 5 minutes of incubation (with occasional tapping to facilitate cell detachment), the trypsin solution was neutralized with equal volume (4 mL) of Trypsin Neutralizer Solution. Cells were then centrifuged twice at 200 g for 5 min after trypsin detachment and neutralization. After the first centrifugation, cells were resuspended in 1 mL PBS. After the second centrifugation, cells were resuspended in appropriate volume of PBS, or a solution of hydrogel composition such as Hydrogel Formulation #6 (1% fibrinogen+1% thrombin) or Formulation #4 (5% gelatin (Sigma) dissolved in PBS and 10 U/g transglutaminase TI). In some embodiments, a final cell density ranging from about 1×10²-1×10¹¹ cells/mL was achieved. In some embodiments, a cell composition also comprises a hydrogel composition such as those selected from Table 1.

### Example 10: Pattern on an Example Supporting Unit

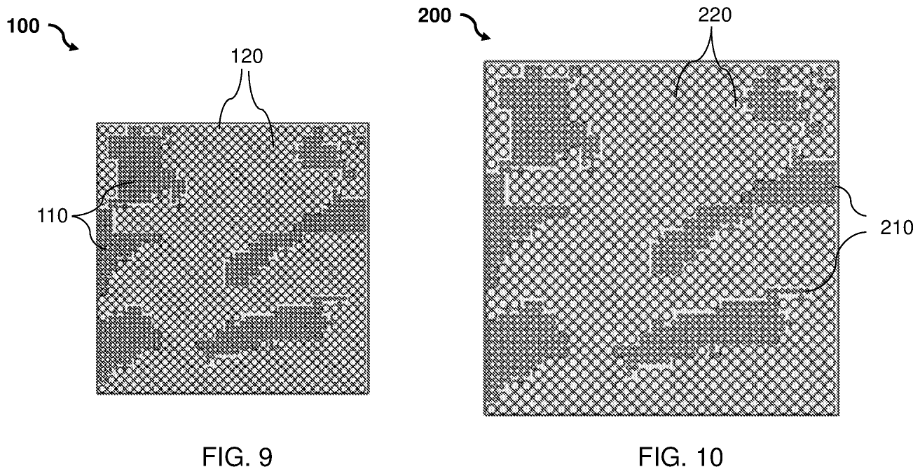

Now referring to FIG. 9, a cross-sectional view of an example design of a desired tissue construct having a pattern 100 with a desired/target muscle fiber/fat fiber dimensions, mimicking the dimensions of a section of an in-vivo Wagyu or Angus cow tissue. In this example, the pattern 100 has an overall dimension of about 5×5 mm. The pattern 100 comprises multiple first cells (muscle) regions arranged in a first pattern formed by a plurality of first channels 110 and multiple second cell (fat) regions arranged in a second pattern formed by a plurality of second channels 120. These patterns are irregularly arranged, mimicking the tissue arrangements of a section of an in-vivo Wagyu or Angus cow tissue. In this example, each of the plurality of first channels 110 has a smaller first diameter of about 60 μm, and the distance between the two adjacent channels is about 30 μm. Each of the plurality of second channels 120 has a larger second diameter of about 135 μm, and the distance between two adjacent second channels is about 30 μm.

Now referring to FIG. 10, a cross-sectional view of the pattern 200 based on the design of the pattern 100. In this example, the pattern 200 has a dimension of about three times larger than that example design pattern 100, i.e., about 15×15 mm. Each of the plurality of first channels 210 has a first diameter of 180 μm, and the distance between two adjacent first channels is about 90 μm. Each of the plurality of second channels 220 has a second diameter of about 405 μm and the distance between two adjacent second channels is about 90 μm. The pattern 200 has an overall dimension (e.g., about 3 times) larger than the desired final dimensions of the pattern 100 to allow production of a cultured tissue in larger dimensions and shrinking in the subsequent steps, so that the final product can achieve the original dimensions of the desired tissue construct after the shrinking step.

### Example 11: Example Device of Producing Cultured Tissues

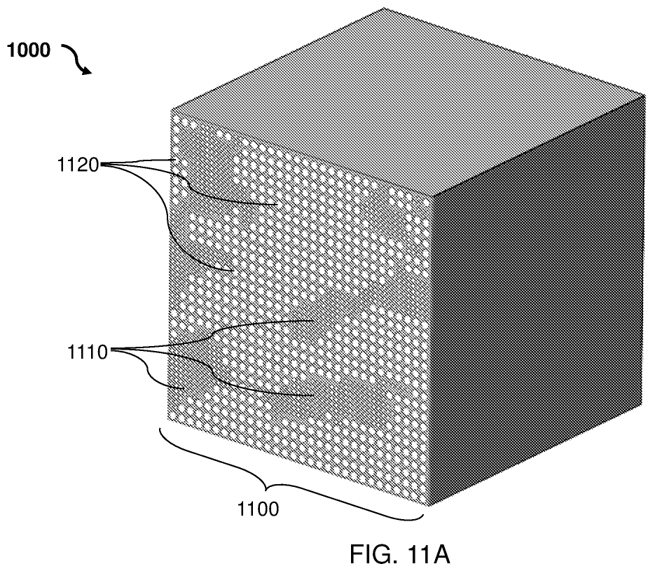

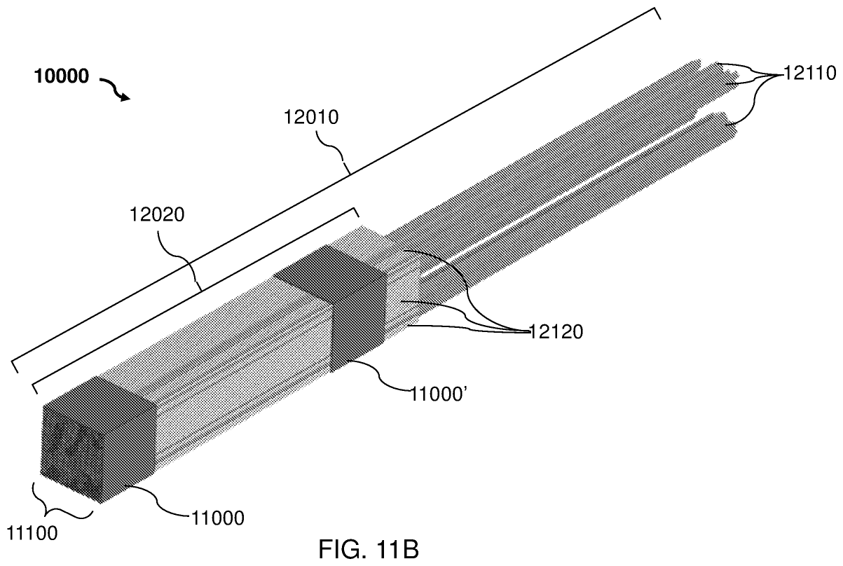

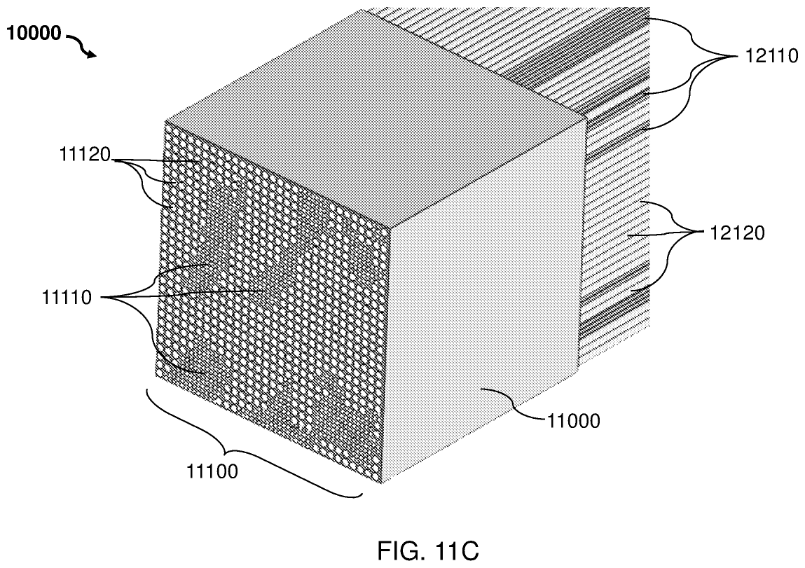

Now referring to FIG. 11A, showing an example supporting unit 1000 (in other examples, also referred to as ‘support plate’ or ‘plate’) including a plurality of first channel receiving portions 1110 (in other examples, also referred to as ‘holes’) and a plurality of second channel receiving portions 1120, the channel receiving portions 1110 and 1120 are constructed and arranged in a first pattern and a second pattern, respectively, similar to the pattern 100 as described in Example 10. Each first channel receiving portion is sized and shaped to receive at least a portion of a first channel forming unit, and each second channel receiving portion is sized and shaped to receive at least a portion of a second channel forming unit, such that a plurality of channel forming units can be assembled and defined by the respective channel receiving portions. In some embodiments, the diameter of each channel receiving portion is slightly larger than the channel forming unit diameter so as to receive at least a portion of a channel forming unit (or needle).

Now referring to FIGS. 11B-11C, showing an example device 10000 (in some examples, also referred to as ‘channel assembly’) having a supporting unit 11000 configured to connect with a proximal side of the holder assembly, supporting unit 11000′ at a distal side of the holder assembly, and a plurality of first channel forming units 12110 and second first channel forming units 12120 (also referred to as ‘first needles’ and ‘second needles’, respectively) are received in respective channel receiving portions, arranged based on the first pattern and second pattern, respectively. In this example, each of the first channel forming units 12110 has a first diameter of a first channel (about 180 μm in this example), and each second first channel forming units 12120 has a second diameter of a second channel (about 405 μm in this example). In this example, each first channel forming units 12110 has a first length 12010 of about 180 mm, and each second channel forming units 12120 has a second length 12020 of about 90 mm.

FIG. 11C is a zoomed-in view of the example device 10000, showing a plurality of first channel receiving portions 11110 and second channel receiving portions 11120 on the supporting units 11000 and 11000′. The plurality of first channel forming units 12110 and second first channel forming units 12120 are arranged based on the pattern 11100 and installed through the respective first and second channel receiving portions 11110 and 11120 such that the plurality of first and second channel forming units 12110 and 12120 can be assembled to form a predetermined first array and second array, respectively.

In some embodiments, the plurality of first channel forming units 12110 and second first channel forming units 12120 were used for muscle fiber formation and fat fiber formation, respectively.

### Example 12: Example Device and Hydrogel Construct

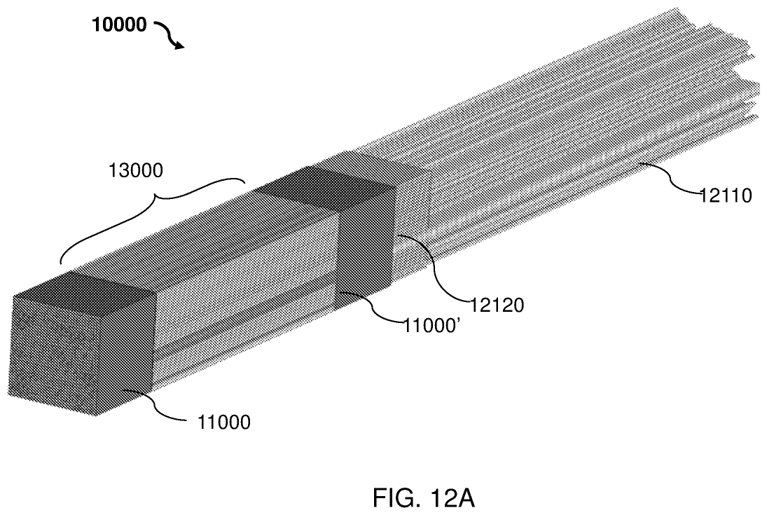

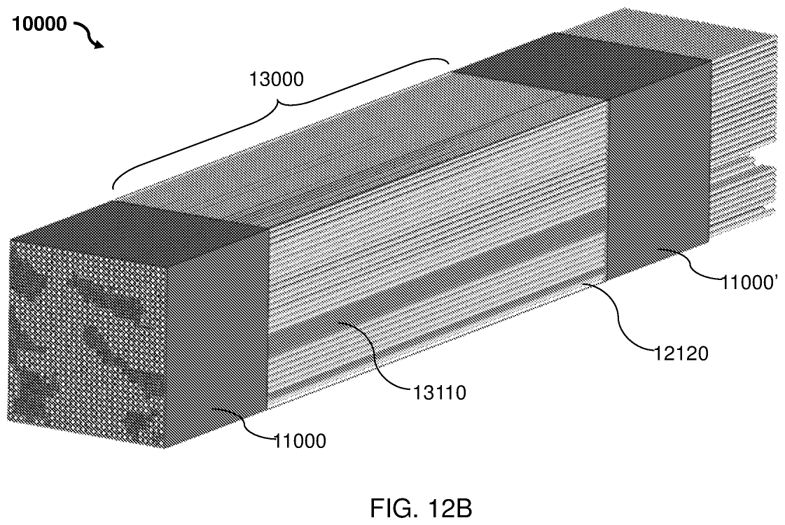

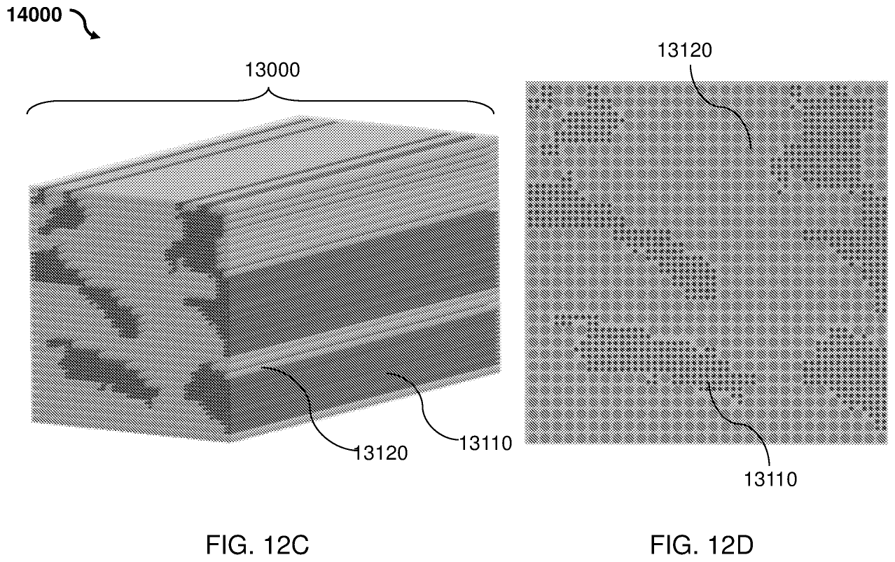

Now referring to FIGS. 12A-C, showing the process of formation of an example device (including hydrogel portion and channel portion). FIG. 12A shows the example device 10000 (as described in Example 11) having a plurality of first channel forming units 12110 and a plurality of second channel forming units 12120 assembled to form a predetermined first array and a predetermined second array, respectively, and a remaining space between, which is defined by the supporting unit 11000, supporting unit 11000′ and where a hydrogel portion 13000 is formed. The hydrogel portion 13000 is formed by pouring a hydrogel composition (such as those according to Table 1) into the remaining space and allowed to be solidified. In this example, a hydrogel composition comprising about 2% sodium alginate and about 10% gelatin (such as Mix A 1 as described in Table 1) was used to form the hydrogel portion 13000.

FIG. 12B shows the example device 10000 (also referred to as ‘channel assembly’) having the hydrogel portion 13000 with the plurality of first channel forming units 12110 removed while the plurality of second channel forming units 12120. In this example, the plurality of first channel forming units 12110 was removed from the hydrogel portion 13000, forming a plurality of first channels 13110 in the predetermined, first array in the hydrogel portion 13000, where individual first channel 13110 is configured to receive at least one first cell composition. In this example, a first cell composition (cell composition B according to Table 2) comprising myoblasts cells (about 1×10⁷ cells/ml) derived from Angus cow suspended in Type 1 collagen (TeloCol®-6 Type I Collagen Solution, 6 mg/ml (Bovine)) was seeded into the first channels 13110 to form a first cell culture. A neutralization solution (10 ml, TeloCol®-6 Type I Collagen Solution, 6 mg/ml (bovine), Advanced BioMatrix) was also added in cell composition B (about 1/10th of the final volume was used) for crosslinking to occur under about 37° C.

In some embodiments, the plurality of second channel forming units 12120 is removed from the hydrogel portion 13000 without a particular order, i.e., separately, before or after the step of removing the first needles, or simultaneously. In this example, the plurality of second channel forming units 12120 was removed from the hydrogel portion 13000 subsequent to the step of obtaining the first cell culture, such that a plurality of second channels (not shown) in the predetermined, second array are formed, where individual second channel is configured to receive at least one second cell composition. In this example, a second cell composition (cell composition C according to Table 2) comprising adipose derived stem cells from Angus cow (1×10⁷ cells/ml) suspended in Type 1 collagen (TeloCol®-6 Type I Collagen Solution, 6 mg/ml (Bovine)) was seeded into the second channels. A neutralization solution was also added in cell composition C (about 1/10th of the final volume was used) for crosslinking to occur under about 37° C.

FIGS. 12C and 12D show an example hydrogel construct 14000 comprising hydrogel portion 13000, first channels 13110 seeded with the first cell composition (comprising muscle derived cells) and second channels 13120 seeded with the second cell composition (comprising fat derived cells), after removing the second first channel forming units 12120 and the two supporting units 11000 and 11000′.

### Example 13: Hydrogel Construct with Perfusion Channels

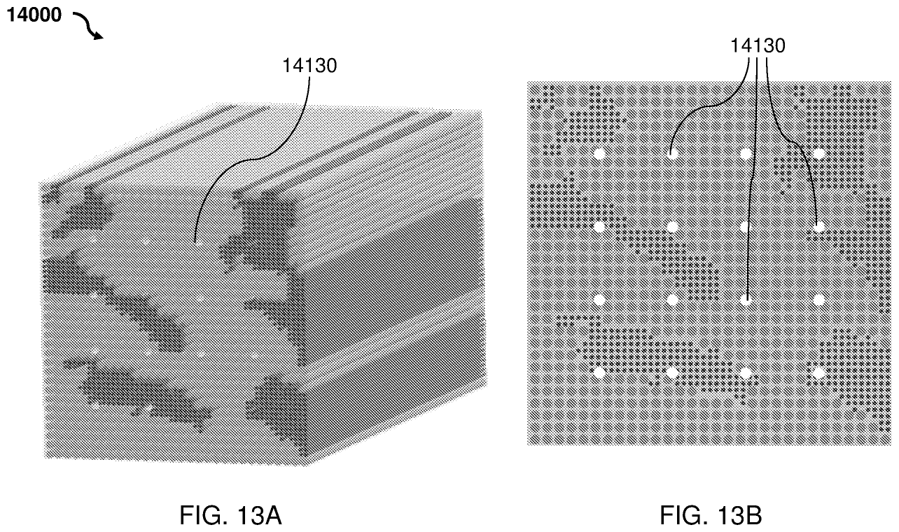

Now referring to FIGS. 13A-13B. FIGS. 13A and 13B show the example hydrogel construct 14000 prepared as described in Example 12, but subsequently being extruded out with a plurality of third channels 14130 (perfusion channels) by biopsy punch tools. In this example, the third channels 14130 formed a 4×4 perfusion third array. In this example, the diameter of each of the third channels 14130 was about 225 μm and the distance between two adjacent third channels is about 3 mm. A cutting tool such as a biopsy punch tool was used to extrude out the third channels 14130. In some embodiments, endothelial cells can be seeded in the third channels 14130 to form endothelial tubes along the surface of the third channels 14130 as perfusion channels.

In another example, the third channels can be created in a hydrogel portion (such as the hydrogel portion 13000) by providing a plurality of third channel forming units together with the first and/or second channel forming units in the device described in Example 11, providing the third cell composition (cell composition D according to Table 2, comprising endothelial cells) in the third channels and letting the third cells to grow. In some embodiments, the third cells comprise endothelial cells, which will grow and differentiate along the surface of the third channels. The third channels are configured so as to fluidly communicate with a perfusion assembly system, which will be described in more details later.

In yet another example, endothelial cells are seeded into the second (or other) channels (such as second channels 13120), and the second (or other) channels could be used for perfusion by connecting with a perfusion assembly system.

### Example 14: Example Hydrogel Construct Adapter and Perfusion Assembly System

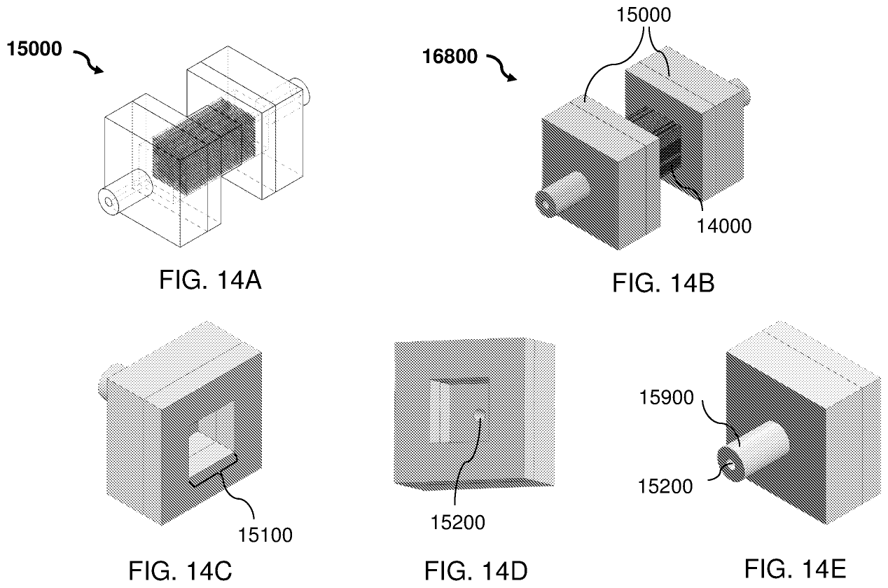

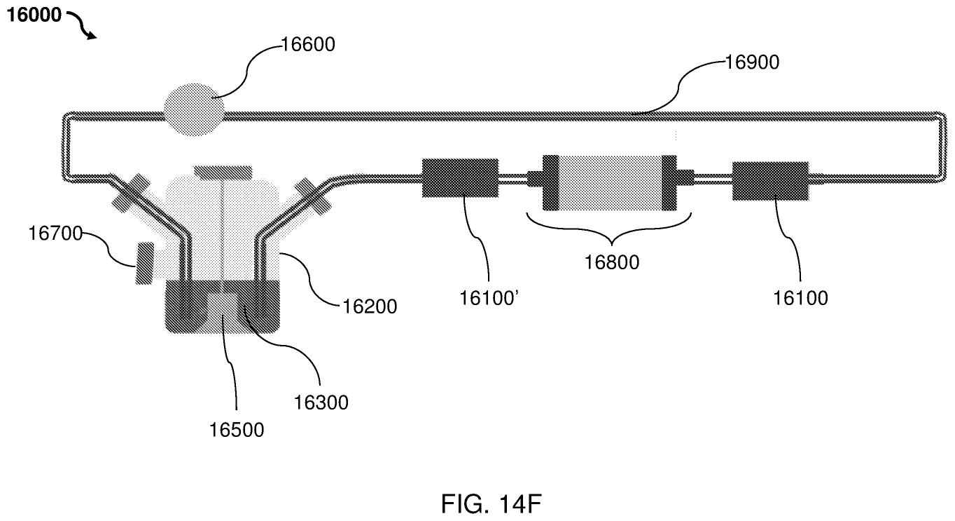

FIGS. 14A and 14B show an example hydrogel construct, such as the hydrogel construct 14000 as described in any one of the examples herein (such as Example 13), sandwiched between two example hydrogel construct adapters 15000 to form an example hydrogel construct assembly 16800.

Now referring to FIGS. 14C-14E, showing illustrations of the example hydrogel construct adapter 15000 at different perspectives. The example hydrogel construct adaptor 15000 includes a hydrogel construct receiving portion 15100, a cavity 15200 and a tubing connection portion 15900. The hydrogel construct receiving portion 15100 is configured to receive at least one end of any one of the hydrogel constructs as described in the preceding examples, such that the perfusion channels (such as third channels 14130 as described in Example 13) are in fluid communication with the cavity 15200. The cavity 15200 extends through the tubing connection portion 15900, which can be connected to the rest of the perfusion assembly system and allows fluid communication therein. In one example, the hydrogel construct receiving portion 15100 is sized and shaped to be larger than a hydrogel construct such that an end portion of the hydrogel construct can be partially fitted in the hydrogel construct receiving portion 15100 while leaving a space between the end portion of the hydrogel portion and the hydrogel construct receiving portion 15100 so that the inlets of the perfusion channels (such as third channels 14130 as described in Example 13) are not blocked, and fluid communication is allowed. In some examples, a sealing step as described in Example 18 can be included to prevent leakage of fluid from the adapter-hydrogel interface.

FIG. 14F shows an example perfusion assembly system 16000 which generally contains a first pump 16100, a second pump 16100′, a hydrogel construct assembly 16800 and a mixer 16200 containing culture medium 16300, stirrer 16500, medium recycler 16600, and vent 16700. These components are connected with each other in fluid communication. A hydrogel construct adapter such as the hydrogel construct adapter 15000 in FIG. 14A (or called tube adapter) is attached to an example meat hydrogel construct (such as the ones described in Example 12) to form a hydrogel construct assembly 16800, and a tubing 16900 is connected with the hydrogel construct assembly 16800 and an oxygenated medium source in fluid communication. In this example, the first and second pumps 16100 and 16100′ are located upstream and downstream of the hydrogel construct assembly 16800, respectively, the mixer 16200 was a spinner bioreactor with a stirrer 16500 to oxygenate medium 16300 and a vent 16700, and the hydrogel construct assembly 16800 had a plurality of perfusion channels (similar or the same as the third channels 14130 discussed in Example 12) connected with the perfusion assembly by the tubing 16900. In this example, the mixer 16200 was a 100 mL bioreactor with a PTFE filter to allow for sterile air to come in and be in contact with the medium 16300. When the medium 16300 was mixed, air (and oxygen) was dissolved to oxygenate the medium. One or more pumps were used to pump the oxygenated medium into the hydrogel construct in the hydrogel construct assembly 16800, and deoxygenated medium with waste from the hydrogel construct. As such, the hydrogel construct is perfused with the medium 16300 (by perfusion step). Optionally, a medium recycler 16600 was connected downstream of the hydrogel construct to remove any waste. Optionally, the hydrogel construct was then allowed to mature to produce a tissue construct as a final cultured meat product.

### Example 15: Final Product of Example Tissue Construct

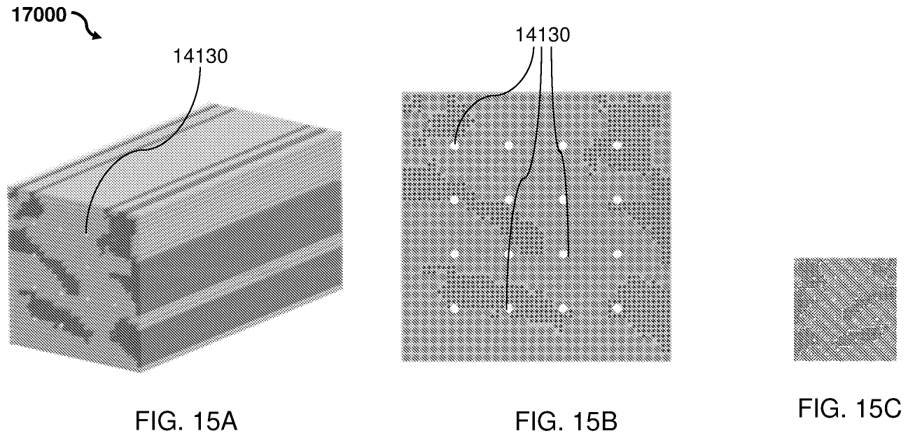

Now referring to FIGS. 15A-15B. FIGS. 15A and 15B show a final target product of example tissue construct 17000 formed after perfusion as described in Example 14, where the plurality of third channels 14130 as described in Example 13 remain present. FIG. 15B shows a cross-sectional view of the tissue product 17000 mimicking Angus cow meat tissue. In this example, the tissue construct 17000 was subsequently treated with a shrinking agent to shrink the tissue. In this example, low molecular weight chitosan (e.g., about 15 kDa) solution was used so the tissue construct 17000 was shrunk by about 3-fold. As such, the tissue construct can be exercised to induce greater matrix re-arrangement and thus, texture. In other examples, the hydrogel construct or tissue construct can be immersed in a solution such as acetic acid (i.e., vinegar) that can shrink the fiber-fiber distance such as to about 20 μm. In other examples, the forces exerted by muscle fibers will cause (such as about 1-20 times, say, about 3 times) shrinkage (may vary depending on the hydrogel used) in diameter to be more similar to a physiologically relevant diameter (50-66.7 μm muscle fiber diameters found in beef). In other examples, the shrinking step can be performed on the hydrogel construct prior to the perfusion step as described in Example 14 (such as the hydrogel construct 14000).

### Example 16: Example Device with Holder Unit

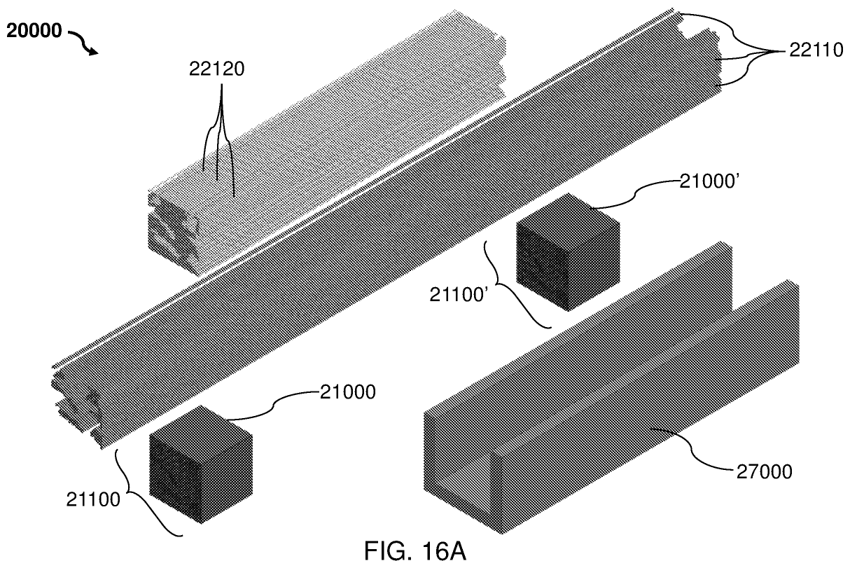

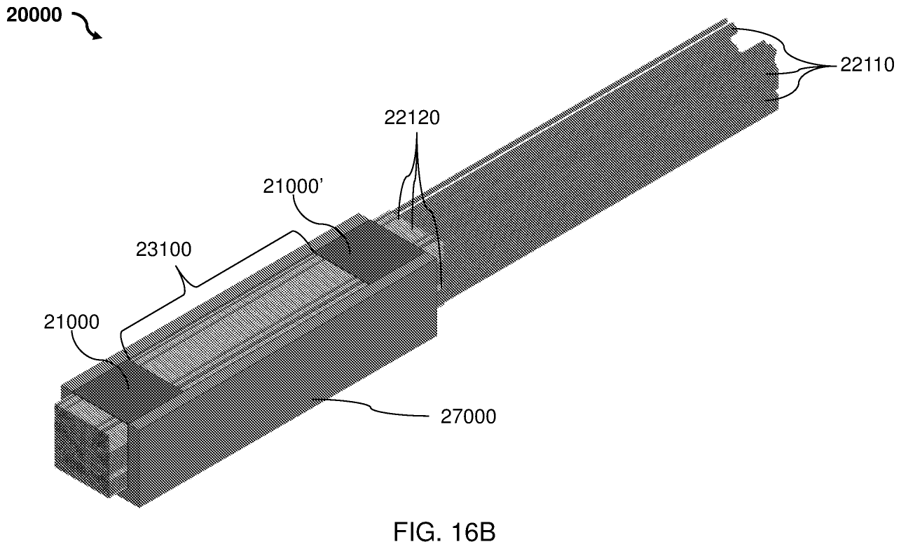

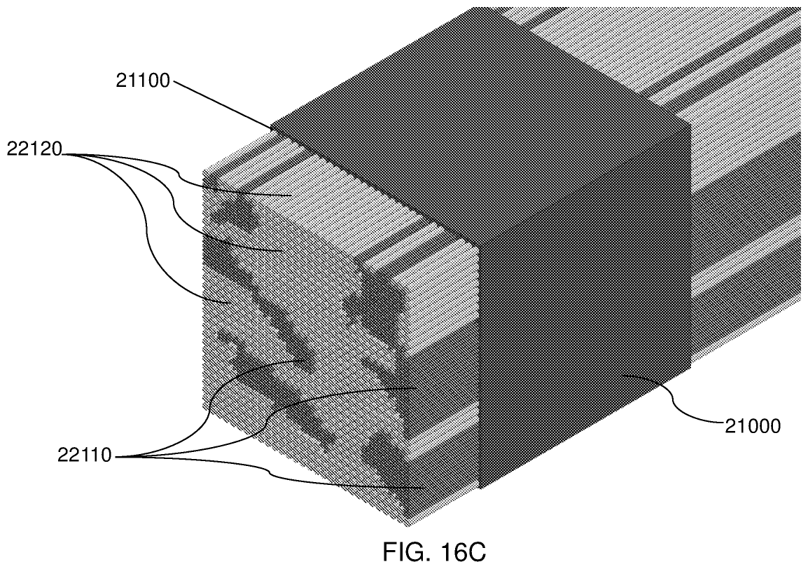

Now referring to FIGS. 16A-16B, showing another example device 20000, which includes supporting unit 21000 at a proximal end of the holder assembly, supporting unit 21000′ at a distal end of the holder assembly, each with a predefined pattern 21100 and pattern 21100′, respectively, a plurality of first channel forming units 22110, a plurality of second channel forming units 22120, and a holder unit 27000. The connections of the two supporting units 21000, 21000′, a plurality of first channel forming units 22110 and a plurality of second channel forming units 22120 are similar to those discussed in Example 11, except the holder unit 27000 is further included, which is sized and shaped to receive the two supporting units 21000, 21000′ and form a holder assembly. Respective channel receiving portions are constructed and arranged in the pattern 21100 such that the plurality of first channel forming units 22110 and second channel forming units 22120 can be assembled with the holder assembly to form a predetermined first array, a predetermined second array, and a remaining space in the holder assembly. In this example, the holder unit 27000 is a U-shaped holder. In this example, the remaining space is a hydrogel receiving portion 23100, where a hydrogel portion of a hydrogel construct can be formed, as shown in FIG. 16B.

FIG. 16C is a zoomed-in view of the channel assembly 20000, showing the plurality of first channel forming units 22110 and the plurality of second channel forming units 22120 partially extending through the plurality of first and second channel receiving portions (not labeled) on the support plate 21000. The plurality of first channel forming units 22110 and the plurality of second channel forming units 22120 are arranged based on the pattern 21100 defined on the supporting units 21000 and 21000′.

### Example 17: Example Device Demonstrating Muscle Fiber Formation

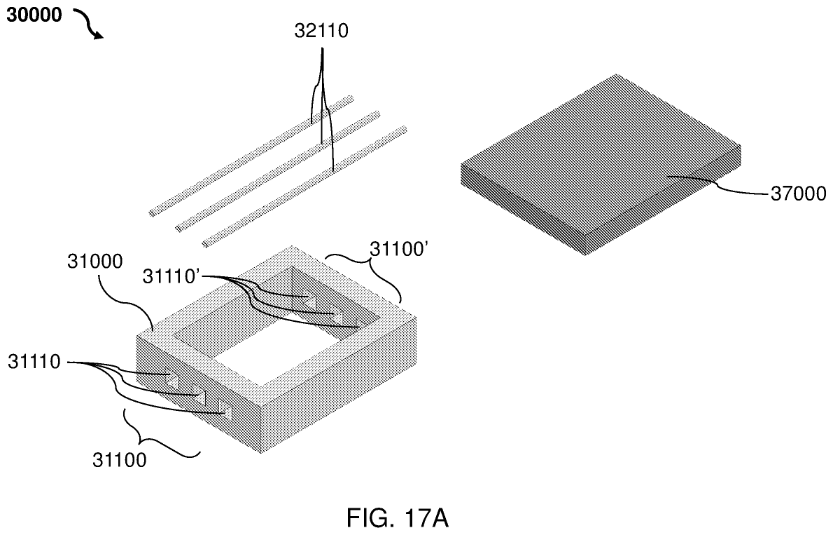

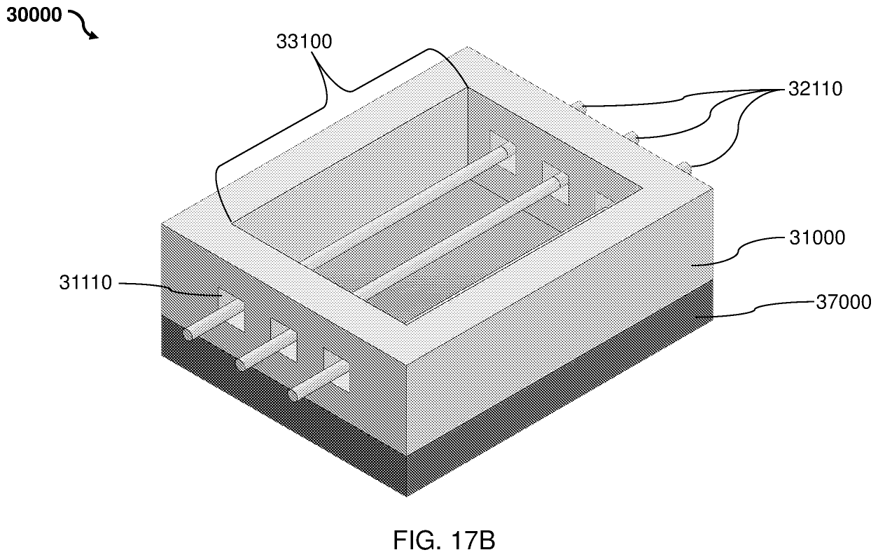

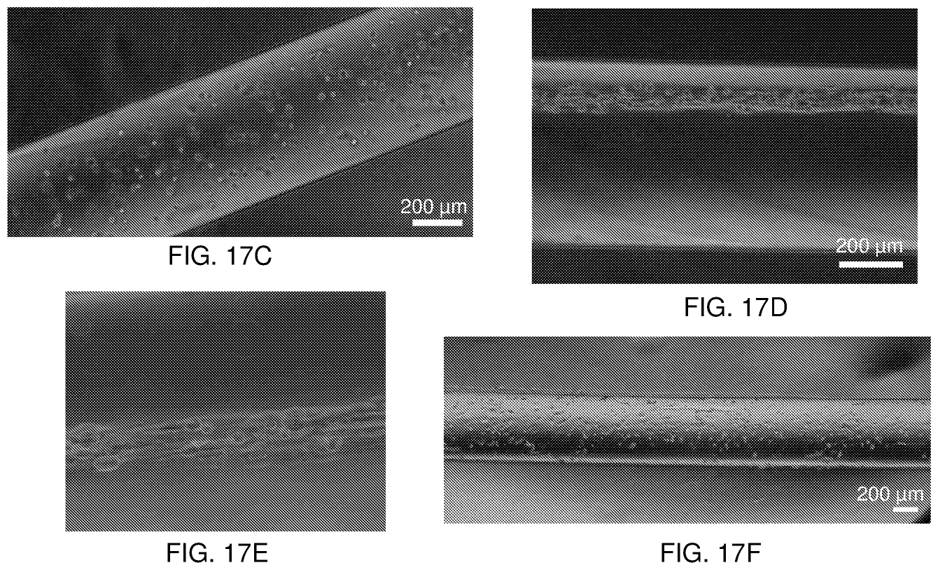

Now referring to FIGS. 17A-17B, showing another example device 30000, which includes a supporting unit 31000, a plurality of channel forming units 32110, and a holder unit 37000. The supporting unit 31000 generally contains two supporting plates having the channel receiving portions 31110 generally parallel to each other which are connected by another two connecting plates, together integrated into one piece as a rectangular frame. The supporting unit 31000 includes a pattern 31100 and a corresponding pattern 31100′ at the opposite sides formed by a plurality of channel receiving portions 31110. In this example, the plurality of channel receiving portions forms a 1×3 matrix as the pattern 31100. The supporting unit 31000 and holder unit 37000 are assembled to form a holder assembly.

Similar to the preceding examples, each channel receiving portion 31110 is sized and shaped to receive at least a portion of a channel forming unit 32110 (such as a needle) and to define the channel forming unit 32110 at its relative position. The diameter of each channel receiving portions 31110 is slightly larger than the channel diameter to receive at least a portion of individual channel forming unit 32110. When the plurality of channel forming units 32110 are arranged based on the pattern 31100 and installed through the respective channel receiving portions 31110 such that the plurality of channel forming units 32110 can be assembled with the holder assembly to form a predetermined first array and a remaining space in holder assembly. In this example, the remaining space is a hydrogel receiving portion 33100 which can receive a hydrogel composition to form a hydrogel portion of a hydrogel construct.

In this example, the holder unit 37000 is a silicone gasket that is sized and shaped to fit the supporting unit 31000.

In this example, the supporting unit 31000 of the device 30000 is fabricated by 3D printing with 3 holes on each side, using poly-lactic acid as the filament for printing. In this example, each hole is about 1 mm in diameter, with the channel-channel distance being about 3 mm.

### Experiment Demonstrating Muscle Fiber Formation

In this example, the device 30000 was used to demonstrate cell growth and alignment within the channels. A hydrogel portion was formed according to the methods described in Example 9a, and the details are described herein.

The components of the device 30000 such as the supporting unit 31000 and holder unit 37000 (also referred to as ‘gasket’) were sterilized and cleaned with detergent, then rinsed in water, wiped dry, and sprayed with ethanol for sterilization. The components were placed in a Biosafety Cabinet and allowed to dry under UV. After drying, the supporting unit 31000 was placed on the holder unit 37000 and pressed hard so there is a slight adhesion therebetween to prevent hydrogel leakage later on.

A plurality of channel forming unit 32110 were sterilized and inserted into the channel receiving portions 31110 of the supporting unit 31000. In this example, the plurality of channel forming units 32110 were 24 G needles (with outer diameter of about 0.566 mm), slightly smaller than the diameter of the channel receiving portions 31110.

Hydrogel composition Formulation #1 as described in Table 1, was prepared according the procedures described in Example 9b, which had a final concentration of about 13.5% gelatin and about 1% transglutaminase TI. The hydrogel composition Formulation #1 was quickly transferred to the device 30000 within about one minute of mixing and allowed to solidify at room temperature. The device 30000 containing the hydrogel composition Formulation #1 was then transferred to a sterile container with basal medium (DMEM: F12) containing about 100 ug/mL Primocin, and the hydrogel composition was allowed to crosslink for 48 hours at room temperature. After 48 hours, a hydrogel portion was formed in the hydrogel receiving portion 33100, and the entire hydrogel construct was incubated in the medium at about 37° C. for about 2 hours.

The medium was subsequently removed as much as possible, then incubate the entire construct at about 75° C. for about 5-10 minutes for inactivation of transglutaminase TI.

The plurality of channel forming units 32110 were removed under a sterile environment such that a plurality of channels (also referred to as ‘microchannels’) in a predetermined array are formed in the hydrogel portion. The hydrogel construct can now be placed in a 6 well plate.

In some embodiments, optionally, the hydrogel construct was sterilized again by ethanol and/or UV.

A cell composition (similar to or the same as one of the examples described in Table 2) was prepared according to the following steps:

Prepared porcine primary myoblast cells by rinsing the myoblasts with sterile PBS (passage 6 or less). In this example, cells from passage 5 were used, followed by the addition of 4 mL of 0.25% Trypsin/EDTA in a T75 flask and incubation for about 5 minutes at about 37° C. The T75 flask was tapped on the sides and bottom occasionally. After 5 minutes of incubation, the Trypsin solution was neutralized with equal volume (4 mL) of Trypsin Neutralizer Solution.

The cell mix was then transferred to a centrifuge tube and centrifuged at 200 g for about 5 minutes. The supernatant was then discarded, and cells were resuspended in PBS. Another centrifuge run at about 200 g for 5 minutes was conducted. The supernatant was once again discarded, and cells were resuspended in 100 μL Formulation #6 prepared according to Example 9b. In this example, the final cell density was about 1×10⁵ cells/mL.

About 20 μL cell composition was immediately injected or introduced into each channel. The entire hydrogel construct was left in a 37° C. incubator for about 30 minutes to complete crosslinking. After sufficient crosslinking, about 5 mL of culture medium (DMEM+10% FBS) was added to the well, and the entire construct was allowed to incubate at about 37° C.

The above preparation process was repeated similarly to prepare a control, where cells without the presence of hydrogel composition Formulation #6 were injected into the channel. After centrifugation, another cell composition was prepared by resuspending the cells in about 100 μL culture medium instead, and the final cell density thereof was about 1×10⁵ cells/mL. About 20 μL of the cell composition in culture medium (without hydrogel) was then injected into each channel. Cells were then allowed to attach in an incubator overnight, with occasional flipping of the channel assembly for attachment throughout the entire microchannel surface.

### Results

By injecting skeletal muscle cells into microchannels (both with and without hydrogels), cells tend to align parallel to their microchannel template. Now referring to FIGS. 17C-17E, microscopic images showing the microchannel seeded with porcine myoblasts within fibrinogen+thrombin gels (i.e., the hydrogel composition Formulation #6) and the resulting muscle fiber formation (scale bar=200 μm). For cells in Formulation #6, cells distributed evenly throughout the microchannels right after being seeded into the microchannels and crosslinking of Formulation #6, as shown in FIG. 17C. Within 1 day of seeding, cells rapidly formed aligned muscle-fibers that were parallel to the microchannel, as shown in FIG. 17D-17E, thereby forming a tissue construct, with some degree of shrinkage in hydrogel being observed. Surprisingly, shrinkage occurred after 1 day in Formulation #6, and the muscle-fiber diameters became much smaller in comparison to the diameter of the microchannel. This could be taken advantage of, in the case that smaller muscle-fibers were desired for greater meat texture. In other examples, manual shrinkage was required via the addition of a shrinking agent.

Now referring to FIG. 17F, showing myoblasts seeded into microchannels without the presence of hydrogel, such as the hydrogel composition Formulation #6. Cells were seen attaching to the walls of the microchannels and tended towards an aligned orientation parallel to the microchannels with a slight degree of confluency, which might still be proliferating and migrating to cover up the empty space.

These results demonstrate that microchannels provided by the example devices can effectively support the formation of cell tissues such as muscle fibers, and that the presence of hydrogel in the cell composition can help facilitate tissue formation (such as regarding cell alignment, confluency and proliferation of cells in the channels, and shrinkages of the cell tissue (such as muscle-fibers), etc). Other example cell compositions having other additives would also improve tissue formation.

### Example 18: Example Device and Example Tissue Construct

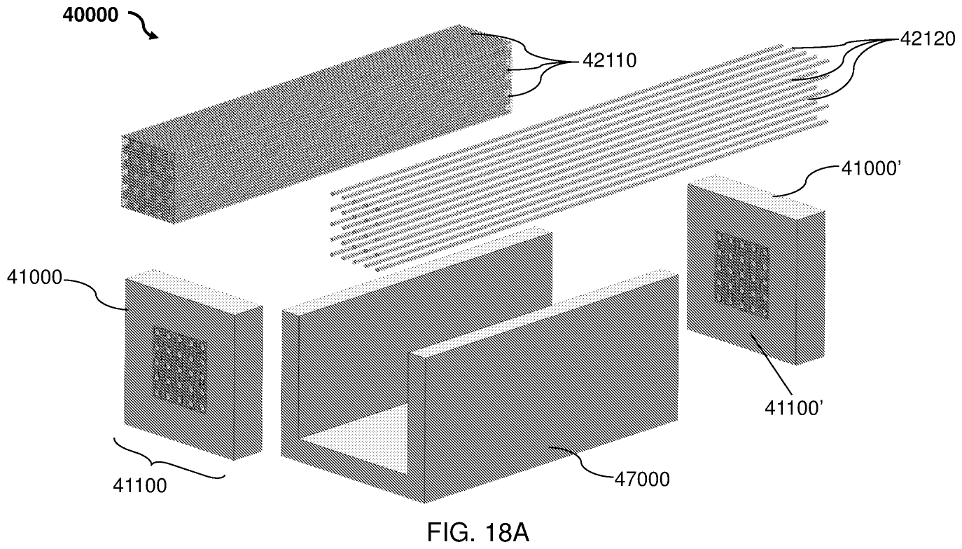

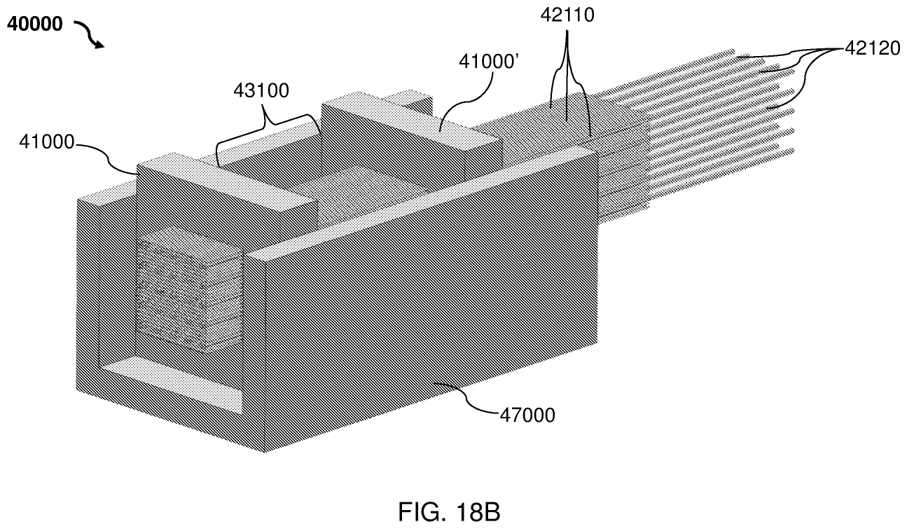

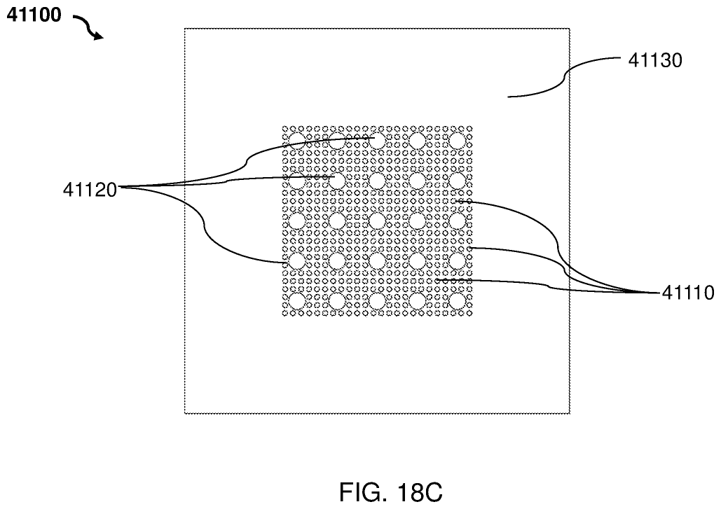

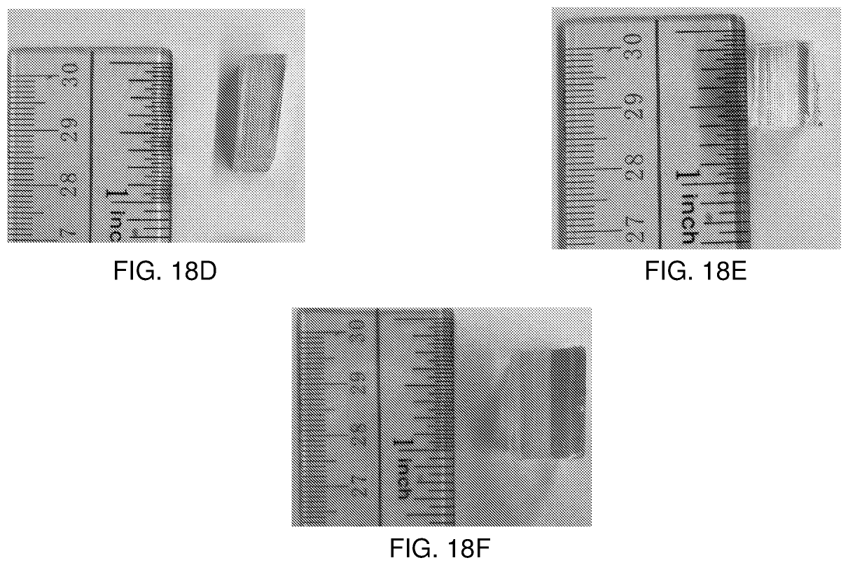

Now referring to FIGS. 18A-18C, showing another example device 40000, which generally includes a supporting unit 41000 at a proximal end of the holder assembly, supporting unit 41000′ at a distal end of the holder assembly, a plurality of first channel forming units 42110, a plurality of second channel forming units 42120, and a holder unit 47000. In this example, the supporting units are in the form of plates. The two supporting units 41000 and 41000′ are sized and shaped to match with the proximal side and distal side of the holder unit 47000, respectively, together forming a holder assembly and define a space therein. The supporting unit 41000 and supporting unit 41000′ are generally in a rectangular shape in the form of a block with a thickness and contains the first channel receiving portions 41110 and second channel receiving portions 41120 in the central area, and an exterior area 41130. The connections of the two supporting units 41000, first channel forming units 42110, second channel forming units 42120 and the holder unit 47000 is the same or similar to the ones discussed in the Example 16, which will not be reproduced herein for the sake of clarity. In the assembled state, respective channel receiving portions 41110 and 41120 on the supporting units 41000 and 41000′ are constructed and arranged in a pattern 41100 and a corresponding pattern 41100′, such that the proximal end and the distal end of each of first channel forming units 42110 can be inserted and received in the respective first channel receiving portions at the proximal side and the distal side, respectively, to form predetermined first array, and the proximal end and the distal end of each of second channel forming units 42110 can be inserted and received in the respective second channel receiving portions at the proximal side and the distal side, respectively, to form predetermined second array, with a remaining space. The remaining space is a hydrogel receiving portion 43100 configured to receive a hydrogel composition to form a solidified, hydrogel portion, such that a plurality of first channels in the predetermined first array are formed by removing the plurality of first channel forming units 42110 from the hydrogel portion, individual first channel is configured to receive at least one first cell composition therein to form at least one first cell culture.

As shown in FIG. 18C, the pattern 41100 is formed by a uniform arrangement of a plurality of first channel receiving portions 41110 and a plurality of second channel receiving portions 41120. In this example, the pattern 41100 is formed by a 24×24 matrix of first channel receiving portions 41110 overlaid with a 5×5 matrix second needle receiving portions 41120. Each of the first channel receiving portion 41110 and the second channel receiving portion 41120 is sized and shaped to receive a first channel forming unit 42110 and second channel forming unit 42120, respectively. In this example, each of the first channel receiving portion 41110 has a diameter of about 250 μm and the distance between two adjacent first channel receiving portions 41110 is about 250 μm (with a center-to-center distance of about 500 μm), and each of the second channel receiving portion 41120 has a diameter of about 550 μm and the distance between two adjacent second channel receiving portions 41120 is about 1450 μm (with a center-to-center distance of about 2000 μm). Each first channel forming unit 42110 has a diameter of a first channel receiving portion 41110 (about 250 μm in this example), and each second channel forming unit 42120 has a diameter of a second channel receiving portion 41120 (about 550 μm in this example).

In this example, an experiment was performed to validate that the fabrication process was consistent and applicable to various hydrogel types, using the example device 40000.

Hydrogel compositions Formulation #2, Formulation #3 and Formulation #1 as described in Table 1 were prepared according to the procedures described in Example 9b.

Similar to the preceding examples, the components of the device 40000 such as the proximal supporting unit 41000 and distal supporting unit 41000′, first channel forming units 42110 and second channel forming unit 42120 were sterilized and transferred into a biosafety cabinet.

The supporting unit 41000 and supporting unit 41000′ were assembled to the holder unit 47000 such that the arrays of holes (e.g., of the pattern 41100) at the proximal side and the distal side were aligned.

In this example, first channel forming units 42110 were elongated wires, each with a about 0.2 mm diameter, and second channel forming units 42120 were elongated wires each with a about 0.6 mm diameter. The plurality of first channel forming units 42110 and second channel forming units 42120 were individually inserted into the supporting unit 41000 and supporting unit 41000′, starting with the first channel forming units 42110, followed by the second channel forming units 42120.

The supporting unit 41000 and supporting unit 41000′ were disposed (pulled away from each another) to the desired plate-plate distance.

The plurality of first channel forming units 42110, second channel forming units 42120 and support plates 41000, were assembled to the holder unit 47000 (also referred to as ‘U-shaped holder’ in this example) to form the example device 40000, as shown in FIG. 18B. The entire device 40000 was sprayed with ethanol and allowed to dry under UV in a biosafety cabinet for sterilization.

Sealing: In this example, a Gelatin Seal Mixture (GSM) was prepared by melting a mixture of about 20% (w/v) gelatin (Aladdin-e) at 37° C. When the example device 40000 was dry, the GSM was transferred into the biosafety cabinet. A small amount (~100 μL per side) of GSM was added to the interface of the U-shaped holder unit 47000 and supporting unit 41000 to create a seal without making contact with the array of holes in the plate, or the wires. The entire device 40000 was tilted when GSM was added, such that the GSM rested horizontally on each side of the interface while solidifying. The GSM seal was then allowed to cool and solidify at room temperature. After the GSM solidified, an additional small amount (~100 μL) of GSM was added to the outer surface of each supporting unit 41000 such that a thin layer was created on the supporting unit 41000 in order to prevent leakage of hydrogel composition from the remaining space via the outflow to the holes later. In this example, the GSM formed a thin (<1 mm) layer of Gelatin on the outer surface of the supporting unit 41000. Drops of hot sterile water (about 50° C.) was slowly added to the wires to melt the residual GSM that leaked into the wires within the channel assembly. Sterile tissue was used to wipe off the residual water.

In some embodiments, the U-shaped holder was closed, and the device was rotated such that GSM can be added to the interface on each side of the supporting unit (i.e., left, right, top and bottom) for proper sealing.

In some embodiments, the sealing steps described above were repeated on the surface of the supporting unit 41000 facing outwards.

In some embodiments, the sealing steps described above were applied to the interface of the U-shaped holder unit 47000 and supporting unit 41000′ as well.

After sealing, the device 40000 was sprayed with ethanol and allowed to dry under UV for 1 hour.

The following steps were carried out when using different hydrogel compositions according to Table 1.

### Formulation #2 as Hydrogel Portion:

Hydrogel composition Formulation #2 was melted in boiling water. To prevent air bubbles from being trapped, the solution was occasionally transferred into the biosafety cabinet, and the cap was opened to release the pressure and bubbles. If air bubbles persisted, the solution was allowed to solidify at 4° C., and then melting was repeated.

The entire device 40000 was placed on ice. The ice facilitated cooling of the mixture rapidly to prevent melting of the gelatin seal (GSM) previously created.

Once the hydrogel composition was sufficiently melted, sufficient volumes were added to the hydrogel receiving portion 43100. The hydrogel composition Formulation #2 was allowed to solidify at 4° C. for 30 minutes and then immersed in 1% calcium chloride for 4 hours for cross-linking to solidify into a hydrogel portion.

### Formulation #3 as Hydrogel Portion:

Hydrogel composition Formulation #3 was melted in 37° C.

The entire device 40000 was placed on ice. The ice facilitated cooling of the mixture rapidly to prevent melting of the gelatin seal previously created.

Once the hydrogel composition was sufficiently melted, sufficient volumes were added to the hydrogel receiving portion 43100. The hydrogel composition Formulation #3 was allowed to solidify at 4° C. for 30 minutes and then immersed in 1% calcium chloride for about 4 hours for cross-linking to solidify into hydrogel portion.

### Formulation #1 as Hydrogel Portion:

The hydrogel composition was prepared as described in Example 9b and used immediately. The mixture was mixed immediately for about 30 s and then added into the hydrogel receiving portion 43100 of the device 40000. The hydrogel composition Formulation #1 was allowed to crosslink in room temperature for about 48 hours, then at 37° C. for about 2 hours. In some embodiments, the mixing and addition of hydrogel composition Formulation #1 to the hydrogel receiving portion 43100 should occur under a minute.

Using a sterile container, the channel assembly containing one of the hydrogel compositions described above was kept under 4° C. for 30 minutes for low-temperature incubation. This step allowed the hydrogel portion being formed to further harden for greater temporary mechanical stability.

To prepare a cell composition, cells of interest were harvested from a passage in a similar manner as described in Example 17. Harvested cells were centrifuged twice after Trypsin detachment and neutralization. After the second centrifuge, cells were resuspended in a solution of Formulation #6. In other examples, cells were resuspended in PBS, sterile water, or any one of the hydrogel compositions as described in Table 1.

In this example, about 1×10⁶ cells/mL of porcine myoblasts were used. In other examples, the final density of cells was at least 1×10⁶ cells/mL.

A small amount (about 100 μL) of the cell composition was added on the interface between the channel forming units 42110, 42120 (i.e., wires), the supporting units 41000, 41000′, such that it completely covers all the first and second channel receiving portions 41110 and 41120 of the supporting units 41000, 41000′. In some embodiments, the device 40000 was rotated vertically rather than horizontally so that the 100 μL volume would not fall out towards one side. When the first channel forming units 42110 were removed or pulled out, the cell composition flowed into the first channels (also referred to as ‘microchannels’). More cell composition was added as needed.

In some embodiments, the first and second channel forming units 42110 and 42120 were removed one-by-one from the example device 40000 and cell composition was introduced.

In some embodiments, cell composition was injected into the microchannel.

In some embodiments, cell composition (containing a hydrogel composition) was allowed to crosslink for about 30 minutes in room temperature.

In some embodiments, the second channel forming units 42120 were removed after cell composition flowed into the first channels in the hydrogel portion formed by the first channel forming units 42110 and solidified.

In some embodiments, the first and second channel forming units 4211 and 4212 (i.e., wires) were removed selectively, and different cell compositions containing different cell types were introduced into the selected channels and allowed to crosslink. In other words, more than one type of first cell compositions and second cell compositions can be introduced sequentially and selectively.

The final construct contains a central area containing the cell compositions and the hydrogel portion therebetween, and an exterior area (or hydrogel exteriors) containing substantially the hydrogel portion only. The exterior area was trimmed, and the central area containing the cell compositions was placed in a petri dish with appropriate amounts of culture medium (in this example, DMEM+10% FBS was used) such that the hydrogel construct was fully immersed therein to grow the cells, thereby forming a tissue construct. The tissue construct was supplied with fresh medium (DMEM+10% FBS) every 2 days.

In some embodiments, other suitable culture media was used to resuspend cells in a cell composition to grow the tissue construct.

### Results

Now referring to FIGS. 18D-18F, showing various perspective of the hydrogel portions having the microchannels before introducing the cell compositions with hydrogel exteriors either trimmed or untrimmed. FIG. 18D is a side view image of a trimmed hydrogel construct prepared using Formulation #2 as the hydrogel portion. When the hydrogel exteriors are further trimmed such that only the central, microchannel sections remain, the hydrogel portion having the microchannel resembles the texture seen in natural meats such as pork. FIG. 18E is a side view image of an untrimmed hydrogel portion prepared using Formulation #3 as the hydrogel portion. FIG. 18F is a side view image of an untrimmed hydrogel portion prepared using Formulation #1 as the hydrogel portion. Results indicated that various hydrogel compositions can successfully form hydrogel portion with microchannels by various example devices with various patterns or array arrangement, various microchannel diameters in the supporting units, and that the microchannels can support the formation of densely packed muscle-fibers. After introducing the cell compositions into the microchannels and incubating the hydrogel construct in suitable conditions to allow the cells to proliferate, differentiate and mature, the microchannels in the hydrogel portion can support the formation of cell tissue (such as muscle fibers), densely packed muscle fibers can be formed, thereby desired tissue construct would be produced, which would resemble a natural tissue construct such as meat.

### Example 19: Example Device with Fasteners and Example Tissue Constructs

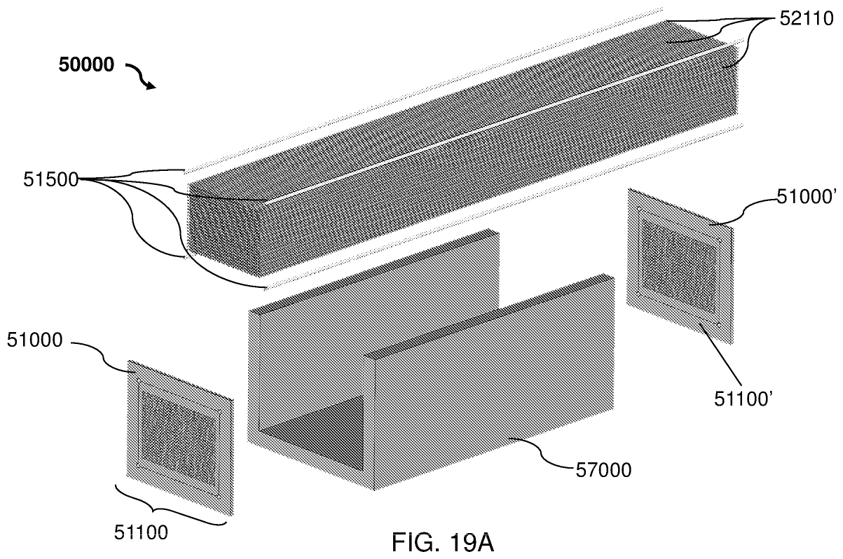

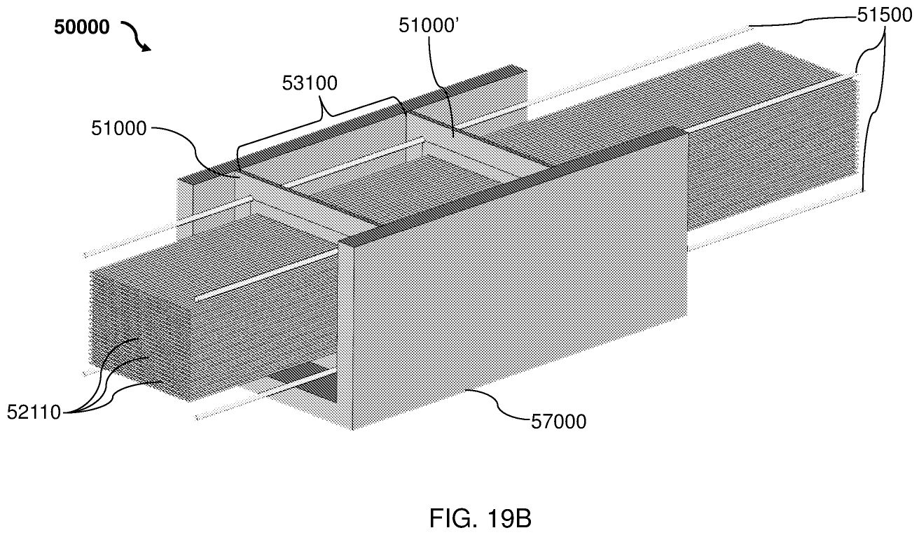

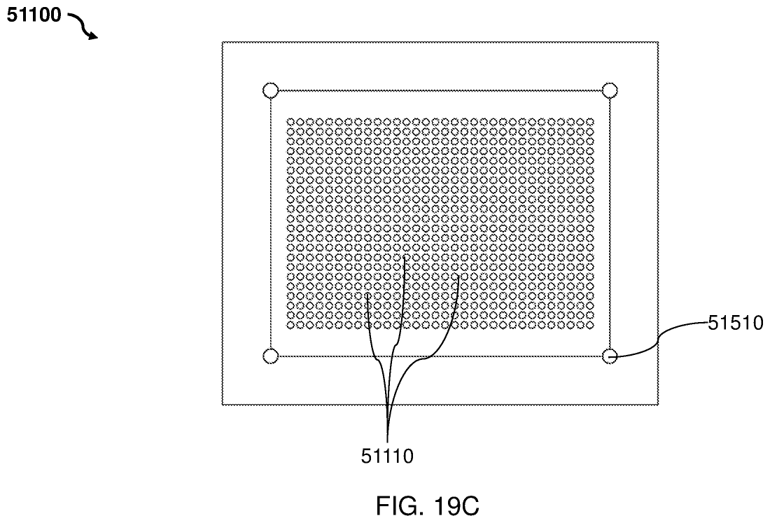

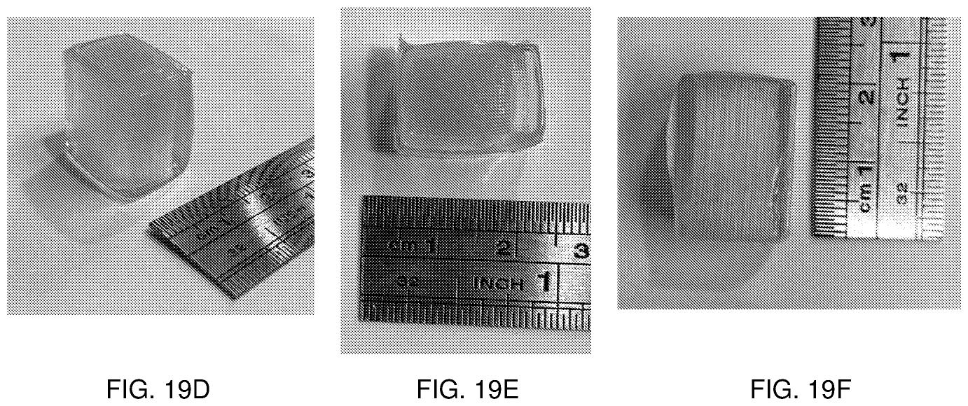

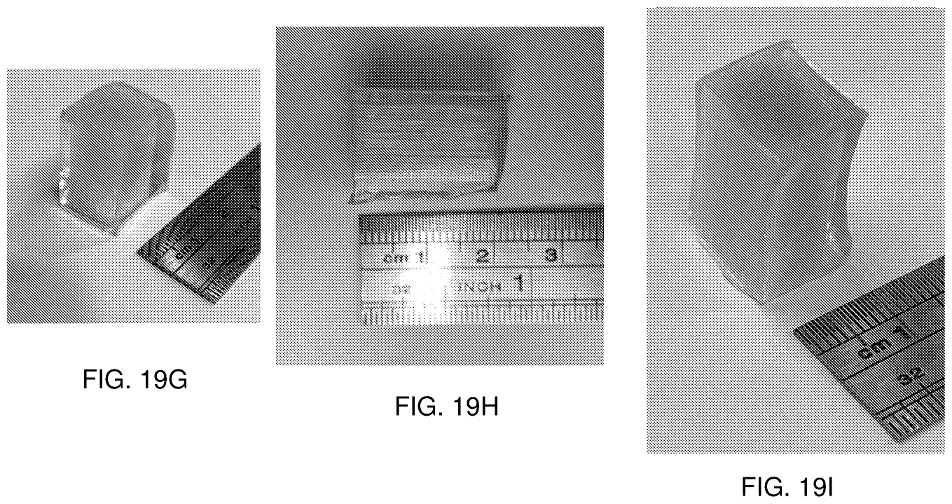

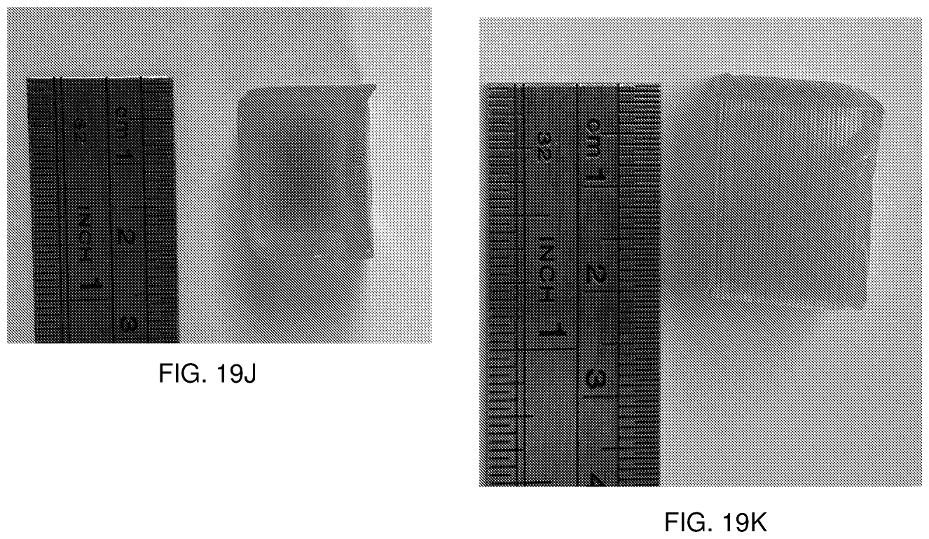

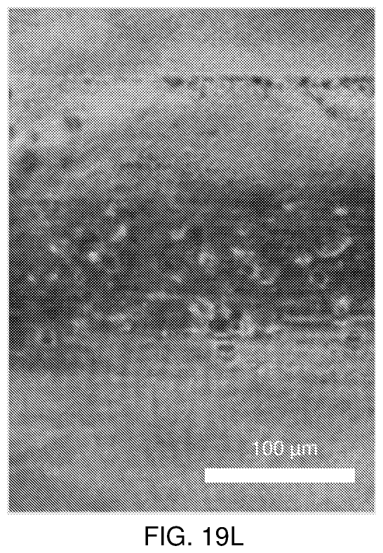

FIGS. 19A-19C show another example device 50000, which generally includes a supporting unit 51000 configured to connect with a proximal side of the holder assembly, supporting unit 51000′ configured to connect with a distal side of the holder assembly, a plurality of supporting unit fasteners 51500, a plurality of channel forming units 52110, and a holder unit 57000. The connections of the supporting unit 51000, supporting unit 51000′, channel forming units 52110, and the holder unit 57000 are similar to those discussed in the Examples 16 and 18, except the device in this example further includes plurality of supporting unit fasteners 51500; the supporting unit 51000 and supporting unit 51000′ both contain respective fastener receiving portions 51510; and the supporting unit 51000 and supporting unit 51000′ are further connected and fixed to each other by installing the plurality of supporting unit fasteners 51500 through the fastener receiving portions 51510. The supporting unit fasteners provide extra support and maintain the relative positions of the supporting unit to the device.

As shown in FIG. 19C, the pattern 51100 is formed by uniform arrangement of a plurality of channel receiving portions 51110. A plurality of channel receiving portions 51110 are constructed and arranged in the pattern 51100 such that the plurality of channel forming units 52110 can be assembled to form a predetermined array and a remaining space. The remaining space is a hydrogel receiving portion 53100 configured to receive a hydrogel composition to form a solidified, hydrogel portion, such that a plurality of channels in the predetermined array are formed by removing the plurality of channel forming units 52110 from the hydrogel portion, individual channel is configured to receive at least one cell composition therein to form at least one cell culture. The supporting units 51000 includes a plurality of fastener receiving portions 51510 surrounding the peripheral of the pattern 51100. The supporting unit 51000′ also includes a plurality of fastener receiving portions surrounding the peripheral of the pattern 51100′. Each of the channel receiving portion 51110 and the fastener receiving portion 51510 is sized and shaped to receive a respective channel forming unit 52110 and supporting unit fastener 51500, respectively. In this example, each channel receiving portion 51110 has a diameter of about 280 μm and the distance between two adjacent channel receiving portion 51110 is about 120 μm, and each fastener receiving portion 51510 has a diameter of about 600 μm, each channel forming unit 52110 has a diameter of a channel receiving portion 51110 (about 280 μm in this example), and each supporting unit fastener 51500 has a diameter of a fastener receiving portion 51510 (about 600 μm in this example).

Now referring to FIGS. 19D-19L, illustrations showing example hydrogel portion prepared using the device 50000 before and after introducing cell compositions. In this example, 1×10⁶ cells/mL of porcine myoblasts in Formulation #6 (1 mg/mL fibrinogen+1 U/mL thrombin) gels are incorporated into 0.2 mm microchannels. FIGS. 19D-19F are isometric view, front cross-sectional view, and side-view images of an example hydrogel portion prepared using Formulation #1 as the hydrogel portion. FIGS. 19G-19H are isometric view and side-view images of an example hydrogel portion prepared using Formulation #3 as the hydrogel portion. FIGS. 19I-19K are isometric view, front view and side-view images showing another hydrogel portion prepared using Formulation #2 as the hydrogel portion. FIG. 19L is a microscopic image showing porcine myoblasts in Formulation #6 introduced into the 0.2 mm microchannels of an example hydrogel construct with Formulation #2 as the hydrogel portion (scale-bar=100 μm). Results indicated that various hydrogel compositions can successfully form hydrogel portion with microchannels by the various example devices with various patterns or array arrangement, various microchannel diameters in the supporting units, and that the microchannels can support the formation of densely packed muscle-fibers. After introducing the cell compositions into the microchannels and incubating the hydrogel construct in suitable conditions to allow the cells to proliferate, differentiate and mature, the microchannels in the hydrogel portion can support the formation of cell tissue (such as muscle fibers), densely packed muscle fibers can be formed, thereby desired tissue construct would be produced, which would resemble a natural tissue construct such as meat.

### Example 20: Reusable Example Device and Methods of Forming Sacrificial Portions

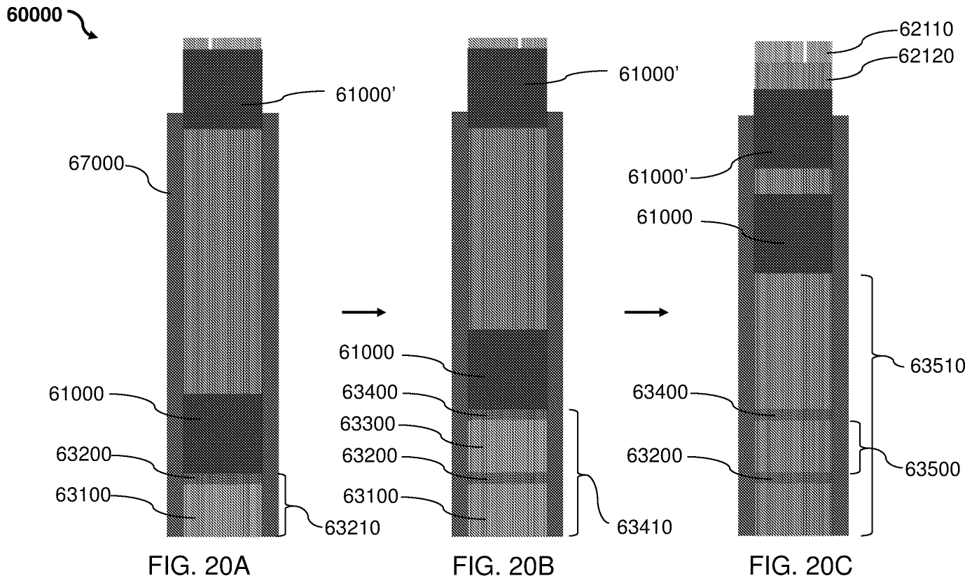

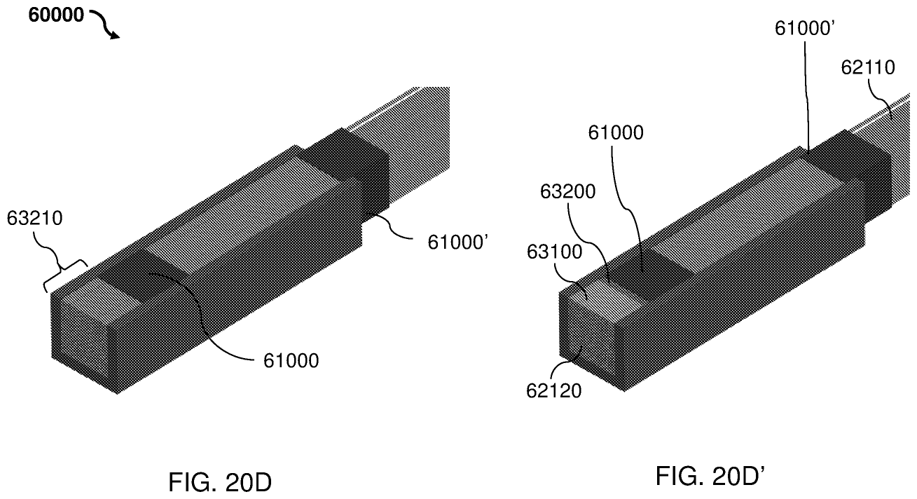

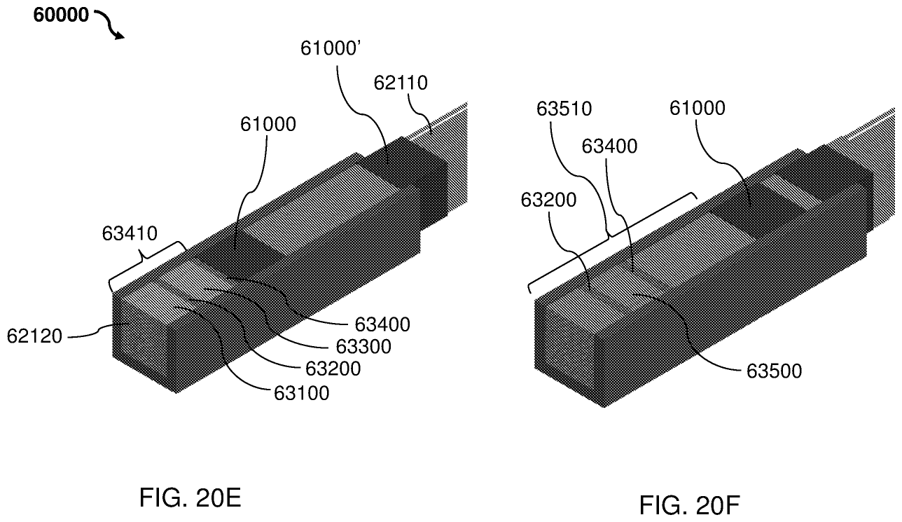

In this example, another example device similar to the ones described in Example 16 was employed to demonstrate that the device can be re-used efficiently without the need of completely disassembling the components (including the first or second channel forming units) from the holder assembly when removing the formed cell construct from the device. Now referring to FIGS. 20A-20F, another example device 60000 is shown, which generally includes supporting unit 61000 at a proximal end of the holder assembly, supporting unit 61000′ at a distal end of the holder assembly, a plurality of first channel forming units 62110, a plurality of second channel forming units 62120, and a holder unit 67000. In this example, 0.2 mm (diameter) wires and 0.4 mm wires were used as the first channel forming units 62110 and the second channel forming units 62120, respectively. In this example, one or more sacrificial portions are provided to serve as temporary supporting units. In some embodiments, the first sacrificial portion 63100 and the second sacrificial portion 63200 were formed in the device 60000 using one or more materials, compounds or compositions that can solidify as a solid or semi-solid, and can be removed (such as melt) under certain conditions, such as water (that can be in the form of ice at low temperatures), or hydrogel compositions (such as any one of the hydrogel compositions described in Table 1).

Methods of forming a hydrogel construct (with sacrificial portions) using the reusable example device 60000 includes the following steps.

Similar to the preceding examples, the two supporting units 61000 and 61000′, plurality of first channel forming units 62110, plurality of second channel forming units 62120, and the holder unit 67000 were assembled to form the device 60000. The device 60000 was sprayed with ethanol and allowed to dry under UV in a biosafety cabinet for sterilization.

When the entire structure was dried, a drop of Gelatin Seal Mixture (GSM) containing 10% (w/v) gelatin (Aladdin-e) was added to the plate-holder interface (for both supporting unit 61000 and 61000′) and allowed to solidify. In some embodiments, the 10% (w/v) gelatin was warmed to about 37° C.

The device 60000 was placed inside a sterile glass bottle (or any other container) vertically. As shown in FIG. 20D, the supporting unit 61000 was disposed at a first position defining a first distance 63210 away from the proximal end of the holder assembly. In this example, the first distance 63210 is about 4 cm. Cold water was poured to the bottom of the glass bottle until the water level was about 2 cm away from the supporting unit 61000′. The glass bottle was closed with a sterile lid and placed in a −20° C. fridge for the water to freeze and form the first sacrificial portion 63100 (as ice). The glass bottle containing the device 60000 was transferred back in the biosafety cabinet. While the supporting unit 61000′ remained in the first position, a about 2-cm layer of GSM was applied on the first sacrificial portion 63100 such that the GSM was making direct contact with the supporting unit 61000. In some embodiments, less GSM could be applied such that it would not be in direct contact with the plate (supporting unit). Allowing the GSM to solidify to form the second sacrificial portion 63200 on the first sacrificial portion. As shown in FIGS. 20A and 20D′, a layer of ice (first sacrificial portion 63100) and GSM (second sacrificial portion 63200) was subsequently formed at the bottom (proximal side) of the example device 60000. The supporting unit 61000 was then separated from the second sacrificial portion 63200 after the GSM solidified.

The supporting unit 61000 was then moved and disposed at a second position defining a second distance 63410 away from the proximal side of the holder assembly. The above steps were repeated such that another layer of ice (third sacrificial portion 63300) and GSM (fourth sacrificial portion 63400) was subsequently formed on the first and second sacrificial portion at the bottom (proximal side) of the of the device 60000, as shown in FIGS. 20B and 20E.

Now referring to FIGS. 20C and 20F, the supporting unit 61000 was then moved and disposed at a third position defining a third distance 63510, away from the sacrificial portions. The device 60000 was then left in room temperature until the ice in the first sacrificial portion 63100 and third sacrificial portion 63300 had melted such that a hydrogel receiving portion 63500 having a defined space was formed, defined by the second sacrificial portion 63200 and fourth sacrificial portions 63400. The device 60000 was then removed from the glass bottle and placed horizontally, sterilized using ethanol and allowed to dry under UV for 1 hour.

Similar to the preceding examples, a hydrogel composition was added to the hydrogel receiving portion 63500. In this example, Formulation #3 as described in Table 1 was used. The device 60000 was immersed in 1% calcium chloride for 4 hours and kept under 4° C. for 30 minutes for low-temperature incubation. A hydrogel portion of a hydrogel construct was formed in the hydrogel receiving portion 63500 between the second sacrificial portion 63200 and fourth sacrificial portion 63400.

To extract the hydrogel portion 63000 from the device 60000, the first channel forming units 62110 and second channel forming units 62120 were pulled past the second sacrificial portion 63200, hydrogel portion 63000, and second sacrificial portion 63200′ while at least a portion of the first channel forming units 62110 and the second channel forming units 62120 still remain assembled with the supporting units 61000 and 61000′. The sacrificial portions can be subsequently removed. As such, the device can be re-used efficiently without the need of completely disassembling the components (including the channel forming units) from the holder assembly when removing the formed construct from the device and re-assembling the same again. In some embodiments, a cell composition or another hydrogel composition (with or without cells could be loaded into the channels within the hydrogel portion 63000 via any method, such as injection, gravitational flow, capillary action, and suction force from the pulling of wires (channel forming units).

In some embodiments, the device 60000 was cleaned by immersing in a solution of 0.2% type II collagenase (to degrade gelatin), 4% sodium citrate (to dissolve alginate hydrogels), and/or in a solution of boiling water, such that the device 60000 was reusable without having to re-assemble the channel forming units 62110 and 62120, and plates 61000 and 61000′. In some other embodiments, the device 60000 was cleaned by autoclaving.

In some embodiments, the device 60000 includes one supporting unit (i.e., supporting unit 61000′) is sufficient because sacrificial portions are prepared as temporary supporting units.

### Example 21: Post-Processing of Example Tissue Construct

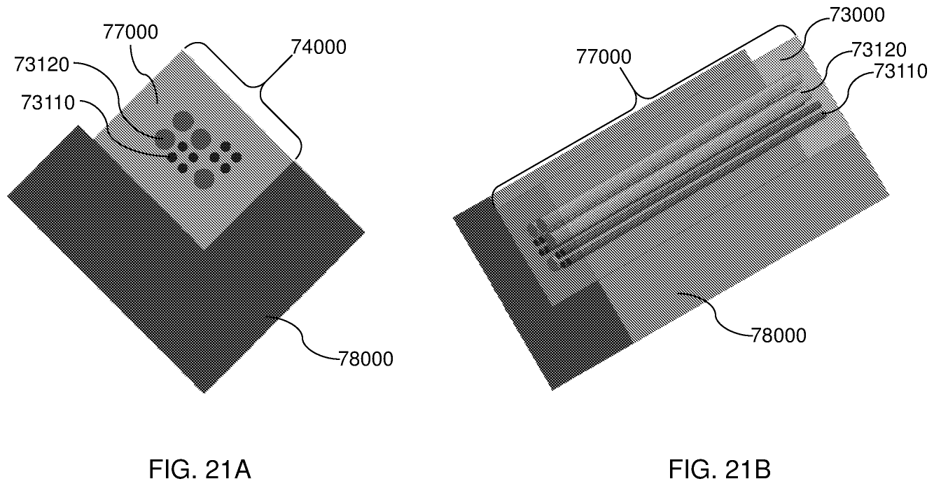

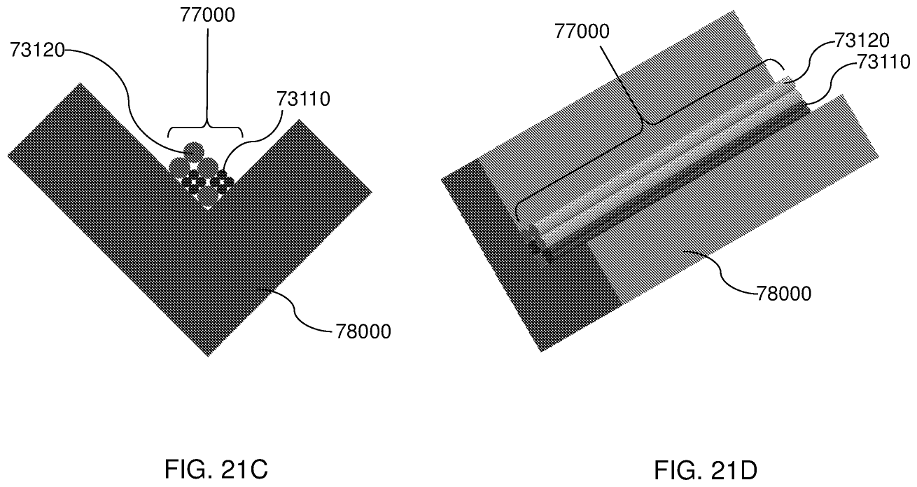

The post-processing treatment of an example tissue construct prepared by the methods described in the preceding examples is described herein. FIGS. 21A-21B showed an example tissue construct 77000 prepared by similar procedures as described in the preceding examples. In this example, the example tissue construct 77000 includes multiple first cell cultures 73110 grown in the first channels, multiple second cell cultures 73120 grown in the second channels (as first and second cell tissues) and a hydrogel portion 73000, disposed between the channels and exterior area. In this example, the hydrogel portion 73000 was formed by using the hydrogel composition Formulation #3 as described in Table 1. In this example, the first cell cultures 73110 were seeded with a first cell composition comprising about 1×10⁷ cells/mL porcine myoblast cells and Formulation #6 to form a first tissue culture in the form of muscle fibers. The second cell cultures 73120 were seeded with a second cell composition comprising about 1×10⁵ cells/mL porcine mature adipocytes and Formulation #6 to form a second tissue culture in the form of fat fibers.

After the formation of the tissue construct 77000, the tissue construct 77000 was placed onto a template 78000, as shown in FIGS. 21A-21B. In some embodiments, the template 78000 was a V-shaped or L-shaped holder rotated diagonally.

The template 78000 containing the tissue construct 77000 was immersed in 4% sodium citrate and transferred to a 37° C. incubator for about 30 minutes. While the hydrogel portion 73000 was being dissolved or melted to remove from the template 78000, the muscle fibers in the first cell cultures 73110 and fat fibers in the second cell cultures 73120 started to settle at the bottom of the template 78000 to form stacked fat and muscle-fibre construct, as shown in FIGS. 21C-21D.

After the hydrogel portion 73000 was melted or dissolved (or removed), the template 78000 containing the remaining tissue construct 77000 was gently aspirated and rinsed with sterile PBS without disturbing the stacked fat and muscle-fibre construct.

In some embodiments, the muscle fibers in the first cell cultures 73110 and fat fibers in the second cell cultures 73120 were then bound together such as by soaking the first cell cultures 73110 and second cell cultures 73120 in about 1% transglutaminase TI solution for about 10 minutes. In other embodiments, a small amount of other binding agents such as a hydrogel composition as described in Example 9b was added to hold the muscle fibers in the first cell cultures 73110 and fat fibers in the second cell cultures 73120 in place.

In some embodiments, the template 78000 contains one or more tissue constructs, such as the example tissue construct 77000, such that the multiple tissue constructs were stacked upon one another after the hydrogel portion 73000 was dissolved or melted, to form a combined tissue construct in larger dimensions.

In some embodiments, the tissue constructs such as meat can undergo further treatment to exercise the muscle tissues (such as by electrical or mechanical means).

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, edible hydrogel has been used in the above embodiments, but non-edible hydrogel may also be used. In some embodiments, non-edible hydrogel may be biodegradable so that the final product contains little or no traces of the inedible hydrogel. In other examples, animal derived hydrogel or non-animal derived hydrogel may be used.

For example, provided devices, systems and methods produce cultured tissue such as cultured meat for consumption, but they can also be used for other applications such as tissue engineering (e.g., for organ or tissue transplantation or replacements). Using similar concepts of vascularization, provided devices, systems and methods can create a thick, living tissue product which is a really hard task to achieve in the tissue engineering industry at the moment.

For example, parameters such as dimensions (such as the channel diameter, channel-channel distance, length), number of needles (and thus channels), distribution of the needle array (e.g., the first array, second array, etc.), size and shape of the needles and support plates, the type of hydrogel use, the type of cells used, for example, fat derived cells in the hydrogel or the channel (e.g. ADSCs, DDFAT), the type of muscle derived cells in the hydrogel or channel (e.g. myoblasts, satellite cells), the type of fibroblasts used (e.g. skeletal muscle fibroblasts, or from other tissue sources), the type of blood vessel cells used (e.g. endothelial cells, pericytes, vascular cells), the type of nerve cells used, the type of tendon cells used (e.g. tenocytes), the type of stem cells used can be adjusted according to the practical need. Other types of cells (e.g., fibroblasts, endothelial cells, blood vessel cells, myoblast cells, muscle cells, fat cells, skin cells, nerve cells, stem cells, tendon, liver, brain, bone, heart, kidney, and combination thereof) used for applications beyond cultured-meat (e.g. for bone tissue engineering, liver tissue engineering etc.), the mixture of cells and hydrogel injected into the channel, the design of the channel (varying channel sizes, sectioning off different channels for either intermuscular fats, intramuscular fats, blood vessels, or a fascicle of muscle tissue), the concentration of hydrogels, the shrinking agent, the way medium is being perfused through the channels or through the resulting blood vessels (e.g. the intensity, duration, frequency, etc. of the pump) can be adjusted according to the practical need.

For example, in certain embodiments as described herein, the cultured tissue is cultured meat, the first cells are muscle derived cells and the second cells are fat derived cells, but in other embodiments, other cultured tissue may be produced, such as organ tissues, blood vessels, tendon cells etc, and other cell types can be used for first cells and second cells, according to the practical need. For example, first cells, second cells and third cells are used to produce the cultured tissue in certain embodiments, but more number of cells (or cell types) (such as fourth, fifth, sixth, seventh, eighth, nineth, tenth cells or more) can additionally or alternatively used.

For example, in certain embodiments as described herein, first channels, second channels and/or third channels are used to produce the cultured tissue, but more number of channels (or respective channel forming units and channel receiving portions) (such as fourth, fifth, sixth, seventh, eighth, nineth, tenth channels or more) can additionally or alternatively used.

For example, needles are used as an example of channel forming units in certain embodiments to create the channels, but the channels may be formed by other means, such as wires, 3D printing, molding, and/or laser ablation.

For example, cell composition is provided by injection, withdrawal, perfusion, or other available means to introduce, seed or deposit a cell composition into a channel.

In some embodiments, hydrogel portion holds the channels within the tissue constructs in place.

In some embodiments, hydrogel portion further includes cells such as fibroblasts, which would secrete extracellular matrix substances to form a network. For example, when the hydrogel portion is melted/dissolved, the network may remain, maintaining the positioning of different tissues within the tissue constructs.

For example, in some embodiments, the methods are performed by using any one of the devices as described herein in any one of the examples. In other embodiments, other devices with other formats, structures and configurations that can provide the first, second and/or third channels in the hydrogel portion to form a tissue construct can also be used.

For example, a tissue construct comprising first cells, second cells and/or third cells are formed, but in other examples, different cell numbers and cell types, different numbers, dimensions, shapes, patterns of channel forming units and channels can be used.

For example, in certain embodiments, channel forming units are elongated, straight wires or needles, but in other examples, other sizes, diameters, shapes and forms of hydrogel forming units can be used. In other examples, the channel forming units are flexible, and can be deformed (such as twisted or compressed) at certain positions when forming the hydrogel construct. For example, a bundle of wires can be provided and compressed when forming the hydrogel portion to form a capillary-like array for seeding endothelial cells, such that the final tissue construct mimics the natural capillary tissues.

For example, in certain embodiments, uniform sized and shaped channel forming units are used for a particular cell type, but in other examples, channel forming units with different sizes, shapes and formats may be used for each type of cells, according to the practical need.

For example, edible hydrogel has been used in the above embodiments, but non-edible hydrogel may also be used. In some embodiments, non-edible hydrogel may be biodegradable so that the final product contains little or no traces of the inedible hydrogel.

For example, provided devices, systems and methods produces cell culture for cultured meat, but they can also be used tissue engineering such as for organ replacements. Using the same concepts of vascularization, provided devices, systems and methods can create a thick, living cell culture which is a really hard task to achieve in the tissue engineering industry at the moment.

For F example, parameters such as dimensions (such as the channel diameter, channel-channel distance, length), number of needles (and thus channels), distribution of the channel array, size and shape of the needles and support plates, the type of hydrogel use, the type of fat cells in the hydrogel or the channel (e.g. ADSCs, DDFAT), the type of muscle cells in the hydrogel or channel (e.g. myoblasts, satellite cells), the type of fibroblasts used (e.g. skeletal muscle fibroblasts, or from other tissue sources), the type of blood vessel cells used (e.g. endothelial cells, pericytes), the type of nerve cells used, the type of tendon cells used (e.g. tenocytes), the type of stem cells used, and other types of cells used for applications beyond cultured-meat (e.g. for bone tissue engineering, liver tissue engineering etc.), the mixture of cells and hydrogel injected into the channel, the design of the channel (varying channel sizes, sectioning off different channels for either intermuscular fats, intramuscular fats, blood vessels, or a fascicle of muscle tissue), the concentration of hydrogels, the shrinking agent, the way media is being perfused through the channels or through the resulting blood vessels (e.g. the intensity, duration, frequency, etc of the pump) can be adjusted according to the practical need.

For example, certain embodiments as described herein, the cultured tissue is cultured meat, the first cells are fat derived cells and the second cells are muscle derived cells, but in other embodiments, other cultured tissue may be produced, such as organ tissues, blood vessels, tendon cells etc., and other cell types can be used for first cells and second cells, according to the practical need. For example, the at least one or more first cells, second cells and/or third cells are derived from fibroblasts, endothelial cells, blood vessel cells, myoblast cells, muscle cells, fat cells, skin cells, nerve cells, stem cells, tendon, liver, brain, bone, heart, kidney, and combination thereof.

For example, needles are used as an example of channel forming units in certain embodiments to create or provide the channels in hydrogel, but the channels may be formed by other means, such as wires, 3D printing, molding, and/or laser ablation etc.

For example, the supporting unit is in the form of a block having a certain thickness to support at least a portion of the channel forming units, but the supporting unit can be in the form of a plate, or stack of a plurality of plates, or in other formats or structures that can support the channel forming units.

For example, in certain embodiments, the first cell tissues (such as muscle fiber) and the second cell tissues (such as fat fiber) are produced simultaneously by the methods, systems and devices, but in other examples, each type of the cell tissues can be produced separately, and the individual cell tissues can be combined in a post-processing step in a desired array.

For example, in certain embodiments, ice or hydrogel is used as a sacrificial layer, but other phase transitional materials may be used instead. In some examples, the phase transitional materials are non-toxic and safe for consumption.

For example, the pattern (or plate design) used contains an array of holes of multiple diameters. For example, different array arrangement of microchannels can be created just by changing the metal plate. For example, an array of uniform and densely packed 0.2 mm microchannels are used.

For example, the exterior area of a hydrogel construct are further trimmed such that only the microchannel sections remain. In some embodiments, the fibrous template resemble the texture seen in traditional meats such as pork.

### NUMBERED EMBODIMENTS

### Numbered Embodiments 1

Embodiment 1. A method of producing cultured tissue, comprising the steps of: (a) providing a hydrogel composition and solidifying the hydrogel composition to form a hydrogel portion comprising a plurality of first channels and a plurality of second channels; (b) providing at least one first cell composition to the plurality of first channels to form at least one first cell culture, and providing with at least one second cell composition to the second channels to form at least one second cell culture, such that that a tissue construct is formed.

Embodiment 2. The method of embodiment 1, wherein the cultured tissue is cultured meat, the first cell composition comprises a plurality of first cells that are muscle derived cells, muscle satellite cells and/or myoblast derived cells and the second cell composition comprises a plurality of second cells that are fat derived cells.

Embodiment 3. The method of any one of embodiments 1-2, wherein the hydrogel composition comprises one or more of alginate, gelatin, gellan gum and combination thereof.

Embodiment 4. The method of any one of embodiments 1-3, wherein the hydrogel composition comprises alginate at about 2% and gelatin at about 10%.

Embodiment 5. The method of any one of embodiments 1-4, wherein the hydrogel composition further comprises fat derived cells and fibroblast cells, and combination thereof.

Embodiment 6. The method of any one of embodiments 1-5, wherein the first cell composition comprises muscle derived cells, muscle satellite cells and/or myoblast derived cells, and optionally one or more of collagen, gellan gum, alginate, gelatin, and combination thereof.

Embodiment 7. The method of any one of embodiments 1-6, wherein the second cell composition comprises fat derived cells, and optionally one or more of collagen, gellan gum, alginate, gelatin, and combination thereof.

Embodiment 8. The method of any one of embodiments 1-7, further comprising the step of: (c) providing a plurality of third channels and optionally providing at least one third cell composition therein.

Embodiment 9. The method of embodiment 8, wherein the third cells comprise fibroblasts, endothelial cells, and combination thereof.

Embodiment 10. The method of any one of embodiments 8-9, wherein the at least one or more first cells, second cells and/or third cells are derived from fibroblasts, endothelial cells, myoblast cells, muscle cells, fat cells, skin cells, tendon, liver, brain, bone, heart, kidney, and combination thereof.

Embodiment 11. The method of any one of embodiments 1 or 8, wherein the plurality of the first, second and/or third channels are formed by removing a plurality of first, second and/or third channel forming units from a solidified hydrogel composition.

Embodiment 12. The method of any one of embodiments 1 or 8, wherein the plurality of the first, second and/or third channels are formed by 3-D printing or laser ablation.

Embodiment 13. The method of embodiment 8, further comprising the step of: (d) growing the first cells and the second cells until a desired tissue mass is obtained, thereby obtaining a cultured tissue product.

Embodiment 14. The method of embodiment 13, wherein the step of (d) is performed by perfusing oxygenated medium through the tissue construct by the plurality of third channels.

Embodiment 15. The method of embodiment 8, further comprising the step of: (e) growing the third cells until a desired tissue mass is obtained.

Embodiment 16. The method of any one of embodiments 8-14, wherein the step (c) is performed after step (b), and wherein the plurality of third channels are formed by extruding out a plurality of perfusion channels by the plurality of third channel forming units.

Embodiment 17. The method of embodiment 16, wherein the plurality of third channel forming units are biopsy punch tools.

Embodiment 18. The method of any one of embodiments 1-17, further comprising the step of: treating the tissue construct with a hydrogel shrinking agent.

Embodiment 19. The method of any one of embodiments 1-18, further comprising the step of: exercising the tissue construct electrically and/or mechanically.

Embodiment 20. The method of any one of embodiments 1-19, wherein prior to the step (b), coating the plurality of first channel forming units and/or the plurality of second channel forming units with lubricant (such as oil).

Embodiment 21. The method of embodiment 18, wherein the hydrogel shrinking agent comprises low molecular weight (e.g., 15 kDa) chitosan.

Embodiment 22. The method of any one of embodiments 1-21, wherein further comprising the step of: crosslinking the hydrogel composition (e.g., with 0.3 M calcium chloride solution).

Embodiment 23. The method of any one of embodiments 1-22, wherein the step of solidifying a hydrogel composition is performed by incubating the hydrogel composition at low temperatures (for example, about 4° C.).

Embodiment 24. The method of any one of embodiments 1-23, wherein the fat derived cells are derived from adipose derived stem cells from cow (such as Wagyu Ribeye).

Embodiment 25. The method of any one of embodiments 1-24, wherein the muscle derived cells are derived from myoblast derived stem cells from cow (such as Wagyu Ribeye).

Embodiment 26. The method of any one of embodiments 1-25, further comprising the step of: differentiating the first cells and/or the second cells into mature cell tissues.

Embodiment 27. A device of producing cultured tissue, comprising: a plurality of first channel forming units, each having a first diameter; a plurality of second channel forming units, each having a second diameter; and at least one support plate, comprising: a plurality of first channel receiving portions, each is sized and shaped to receive at least a portion of a first channel forming units and to define the first needle at a first position; and a plurality of second channel receiving portions, each is sized and shaped to receive at least a portion of a second channel forming units and to define the second needle at a second position, wherein the plurality of first channel receiving portions and the plurality of second channel receiving portions are constructed and arranged in an array such that the plurality of first channel forming units and the plurality of second channel forming units can be assembled together by the at least one support plate to form a channel array and a plurality of spaces therebetween, wherein the plurality of spaces are configured to receive a hydrogel composition to form a solidified, hydrogel portion, such that a plurality of first channels and a plurality of second channels are formed by removing the plurality of first channel forming units and the plurality of second channel forming units from the hydrogel portion, respectively, each first channel is configured to receive at least one first cell composition therein to form at least one first cell culture and each second channel is configured to receive at least one second cell composition therein to form at least one second cell culture, thereby a tissue construct is produced.

Embodiment 28. The device of embodiment 27, further comprising a plurality of third channel forming units to form a plurality of third channels for receiving at least one third cells therein.

Embodiment 29. The device of embodiment 27, wherein the first diameter and the second diameter are larger (e.g., about 1-20 times larger) than diameters of target first cell culture and target second cell culture respectively.

Embodiment 30. The device of embodiment 27, wherein the first cells composition comprises at least one first cell, and the second cell composition comprises at least one second cells.

Embodiment 31. The device of any one of embodiments 27-30, wherein the cultured tissue is cultured meat, the first cells are muscle derived cells, muscle satellite cells and/or myoblast derived cells and the second cells are fat derived cells.

Embodiment 32. The device of any one of embodiments 27-31, wherein the first diameter is about 0.1-10,000 μm, for example, about 20-500 μm.

Embodiment 33. The device of any one of embodiments 27-31, wherein the first diameter is less than 200 μm, for example, 20-90 μm.

Embodiment 34. The device of any one of embodiments 27-31, wherein a distance between two adjacent first channel forming units is about 0.1-5,000 μm, for example, 0.1-500 μm.

Embodiment 35. The device of any one of embodiments 27-31, wherein the distance between two adjacent first channel forming units is less than 100 μm, for example, 0.1-30 μm.

Embodiment 36. The device of any one of embodiments 27-31, wherein the second diameter is about 0.1-10,000 μm, for example, about 20-500 μm.

Embodiment 37. The device of any one of embodiments 27-31, wherein the second diameter is less than 500 μm, for example, 100-300 μm.

Embodiment 38. The device of any one of embodiments 27-31, wherein a distance between two adjacent second channel forming units is about 0.1-5,000 μm, for example, 0.1-500 μm, for example, about 1 μm.

Embodiment 39. The device of embodiment 38, wherein the distance between two adjacent second channel forming units is less than 100 μm, for example, 0.1-30 μm.

Embodiment 40. The device of any one of embodiments 28-39, wherein the third cells comprise at least one or more of fibroblasts, endothelial cells, myoblast cells, muscle derived cells, fat derived cells, and combination thereof.

Embodiment 41. The device of any one of embodiments 27-40, wherein individual of the plurality of first and/or second channel forming units has a cylindrical, tubular structure.

Embodiment 42. The device of any one of embodiments 27-41, wherein individual of the plurality of first and/or second channel forming units has an open, trough structure with a U-shaped cross-section.

Embodiment 43. The device of any one of embodiments 27-42, wherein the tissue construct comprises a plurality of fat regions and a plurality of muscle regions.

Embodiment 44. A system of producing cultured tissue, comprising: a device as claimed in any one of the preceding embodiments to produce a tissue construct having a plurality of third channels; a tissue construct adapter, configured to connect with the tissue construct; a bioreactor for oxygenating a medium; at least one pump system for circulating the medium from the bioreactor to the tissue construct through the plurality of third channels; and optionally a media recycler to remove waste.

Embodiment 45. The system of embodiments 44, wherein the cultured tissue is cultured meat, the first cells are muscle derived cells, muscle satellite cells and/or myoblast derived cells and the second cells are fat derived cells.

Embodiment 46. A cultured tissue prepared by any one of the methods of any one of embodiments 1-26, wherein the cultured tissues are derived from animals selected from the group consisting of mammals, birds, fish, invertebrates, reptiles, and amphibians.

Embodiment 47. The cultured tissue of embodiment 46, wherein the animals are non-human.

Embodiment 48. The cultured tissue of embodiment 46, wherein the cultured tissue is cultured meat.

Embodiment 49. The cultured tissue of embodiment 46, wherein the animals are human.

### Numbered Embodiments 2

Embodiment 1. A method of producing cultured tissue, comprising the steps of: (a) providing a hydrogel composition and solidifying the hydrogel composition to form a hydrogel portion, wherein the hydrogel portion comprises a plurality of first channels in a predetermined first array; (b) providing at least one first cell composition to individual first channel to form at least one first cell culture therein, such that that a tissue construct is formed.

Embodiment 2. The method of embodiment 1, wherein the hydrogel portion further comprises a plurality of second channels in a predetermined second array, and the method further comprises the step of: (c) providing at least one second cell composition to individual second channel to form at least one second cell culture therein.

Embodiment 3. The method of any one of the preceding embodiments, wherein the cultured tissue is cultured meat, the first cell composition comprises a plurality of first cells that are muscle derived cells, muscle satellite cells and/or myoblast derived cells; and the second cell composition, if present, comprises a plurality of second cells that are fat derived cells.

Embodiment 4. The method of any one of the preceding embodiments, wherein the hydrogel composition comprises a first compound selected from a group consisting of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combination thereof, and optionally a second compound selected from a group consisting of transglutaminase, thrombin, tyrosinase, and combination thereof.

Embodiment 5. The method of any one of the preceding embodiments, wherein the hydrogel composition comprises any one of the following formulations: Type 1 collagen solution of about 0.1-20 mg/ml; gelatin of about 0.5-50% (w/v) and gellan gum of about 0.01-5% (w/v); gelatin of about 0.5-50% (w/v) and sodium alginate of about 0.1-6% (w/v); gelatin of about 0.5-50% (w/v) and transglutaminase of about 0.1-100 U per gram of gelatin; and/or fibrinogen of about 0.01-50 mg/mL and thrombin of about 0.01-100 U/mL.

Embodiment 6. The method of any one of the preceding embodiments, wherein the hydrogel composition further comprises fat derived cells and fibroblast cells, and combination thereof.

Embodiment 7. The method of any one of the preceding embodiments, wherein the at least one first cell composition comprises muscle derived cells, muscle satellite cells and/or myoblast derived cells, and optionally one or more of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combination thereof.

Embodiment 8. The method of any one of the preceding embodiments, wherein the at least one second cell composition comprises fat derived cells, and optionally one or more of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combination thereof.

Embodiment 9. The method of any one of the preceding embodiments, wherein the hydrogel portion further comprises a plurality of third channels as perfusion channels, and the method optionally further comprises the step of: (d) providing at least one third cell composition to individual third channel to form at least one third cell culture therein.

Embodiment 10. The method of embodiment 9, wherein the third cell composition comprises fibroblasts, endothelial cells, smooth muscle cells, pericytes, and combination thereof.

Embodiment 11. The method of any one of the preceding embodiments, wherein the plurality of first cells, second cells and/or third cells, if present, are derived from fibroblasts, endothelial cells, blood vessel cells, myoblast cells, muscle cells, fat cells, skin cells, nerve cells, stem cells, tendon cells, liver cells, brain cells, bone cells, heart cells, kidney cells, nerve cells, and combination thereof.

Embodiment 12. The method of any one of the preceding embodiments, wherein the at least one first cell composition, the at least one second cell composition and/or the at least one third cell composition, if present, further comprise any one of the following formulations: Type 1 collagen solution of about 0.1-20 mg/ml; gelatin of about 0.5-50% (w/v) and gellan gum of about 0.01-5% (w/v); gelatin of about 0.5-50% (w/v) and sodium alginate of about 0.1-6% (w/v); gelatin of about 0.5-50% (w/v) and transglutaminase of about 0.1-100 U per gram of gelatin; and/or fibrinogen of about 0.01-50 mg/mL and thrombin of about 0.01-100 U/mL.

Embodiment 13. The method of any one of embodiments 5 or 12, wherein the hydrogel composition, or the at least one first cell composition, the at least one second cell composition and/or the at least one third cell composition, if present, comprise any one of the following formulations: Type 1 collagen solution of about 6 mg/ml; gelatin of about 20% (w/v) and gellan gum of about 2% (w/v); gelatin of about 20% (w/v) and sodium alginate of about 2% (w/v); gelatin of about 13.5% (w/v) and transglutaminase of about 1 U per gram of gelatin; and/or fibrinogen of about 1 mg/mL and thrombin of about 1 U/mL.

Embodiment 14. The method of any one of the preceding embodiments, wherein the individual first channel and/or individual second channel, if present, are formed by the following steps: (1) providing a device of producing cultured tissue comprising a plurality of first channel forming units, and/or second channel forming units and a holder assembly comprising: a holder unit; and at least one supporting unit, each comprising a plurality of first channel receiving portions, wherein individual first channel receiving portion is sized and shaped to receive at least a portion of an individual first channel forming unit, and wherein the plurality of first channel receiving portions are constructed and arranged in a first pattern such that the plurality of first channel forming units can be assembled with the holder assembly to form a predetermined first array and a remaining space in the holder assembly; (2) providing a hydrogel composition to at least a portion of the remaining space and solidifying the hydrogel composition to form a hydrogel portion; and (3) removing individual first channel forming unit and/or second channel forming unit from the hydrogel portion.

Embodiment 15. The method of embodiment 14, prior to step (2), further comprising the following steps: (i) providing a second sacrificial layer in the holder assembly; (ii) providing a third sacrificial layer on the second sacrificial layer; (iii) providing a fourth sacrificial layer on the third sacrificial layer; (iv) removing the third sacrificial layer, such that the second sacrificial layer and the fourth sacrificial layer defines the remaining space for hydrogel portion within the holder assembly.

Embodiment 16. The method of embodiment 15, prior to step (i), further comprising the step of: providing a first sacrificial layer in the holder assembly, such that the second sacrificial layer is provided on the first sacrificial layer.

Embodiment 17. The method of embodiment 15 or 16, wherein step (i) further comprises the steps of: disposing the supporting unit at a first position proximate to the second sacrificial layer; and wherein step (iii) further comprising step of: disposing the supporting unit at a second position proximate to the fourth sacrificial layer.

Embodiment 18. The method of embodiment 15, wherein the first sacrificial layer, the second sacrificial layer, the third sacrificial layer, and/or the fourth sacrificial layer comprises ice and/or hydrogel.

Embodiment 19. The method of embodiment 17, wherein the first sacrificial layer, if present, comprises ice, the second sacrificial layer comprises hydrogel, the third sacrificial layer comprises ice and/or the fourth sacrificial layer comprises hydrogel.

Embodiment 20. The method of any one of the preceding embodiments, wherein the individual first channel, second channel and/or third channel, if present, are formed by 3-D printing and/or laser ablation.

Embodiment 21. The method of any one of the preceding embodiments, further comprising the step of: (e) growing the plurality of first cells, the plurality of second cells and/or the plurality of third cells, if present, until a desired tissue mass is obtained.

Embodiment 22. The method of embodiment 21, wherein the step of (e) is performed by perfusing an oxygenated medium through the tissue construct by the plurality of third channels.

Embodiment 23. The method of any one of embodiments 21-22, wherein the step (e) is performed after step (be) or step (c), and wherein the plurality of third channels are formed by extruding out at least a portion of the hydrogel portion as perfusion channels by a plurality of third channel forming units.

Embodiment 24. The method of embodiment 23, wherein individual third channel forming unit is a cutting tool (such as a biopsy punch tool).

Embodiment 25. The method of any one of the preceding embodiments, further comprising the step of: (f) treating the tissue construct with a hydrogel shrinking agent.

Embodiment 26. The method of any one of the preceding embodiments, further comprising the step of: (g) exercising the tissue construct electrically and/or mechanically.

Embodiment 27. The method of any one of the preceding embodiments, wherein prior to the step (2), coating individual first channel forming unit, second channel forming unit and/or third channel forming unit, if present, with a lubricant (such as oil).

Embodiment 28. The method of embodiment 25, wherein the hydrogel shrinking agent comprises low molecular weight (e.g., 15 kDa) chitosan.

Embodiment 29. The method of any one of the preceding embodiments, wherein the step of solidifying the hydrogel composition is performed by: crosslinking the hydrogel composition with a crosslinking agent (e.g., about 0.03-1% calcium chloride solution); and/or incubating the hydrogel composition at low temperatures (e.g., about 4° C.).

Embodiment 30. The method of any one of the preceding embodiments, wherein the fat derived cells are derived from adipose derived stem cells from cow (such as Angus cows).

Embodiment 31. The method of any one of the preceding embodiments, wherein the muscle derived cells are derived from myoblast derived stem cells or muscle satellite cells from cow (such as Angus cows).

Embodiment 32. The method of any one of the preceding embodiments, further comprising the step of: differentiating the first cells, the second cells and/or the third cells, if present, into mature, cell tissues.

Embodiment 33. The method of any one of the preceding embodiments, wherein the first cell composition comprises porcine myoblast cells.

Embodiment 34. A device of producing cultured tissue, comprising: a plurality of first channel forming units, individual first channel forming unit having a first diameter; and a holder assembly, comprising a holder unit; and at least one supporting unit, each comprising a plurality of first channel receiving portions, wherein individual first channel receiving portion is sized and shaped to receive at least a portion of an individual first channel forming unit, and wherein the plurality of first channel receiving portions are constructed and arranged in a first pattern such that the plurality of first channel forming units can be assembled with the holder assembly to form a predetermined first array and a remaining space in the holder assembly, and wherein at least a portion of the remaining space is configured to receive a hydrogel composition to form a solidified, hydrogel portion, such that a plurality of first channels in the predetermined, first array are formed by removing the plurality of first channel forming units from the hydrogel portion, individual first channel is configured to receive at least one first cell composition therein to form at least one first cell culture, thereby a tissue construct is produced.

Embodiment 35. The device of embodiment 34, further comprises a plurality of second channel forming units, individual second channel forming unit having a second diameter, wherein the at least one supporting unit further comprises a plurality of second channel receiving portions, individual second channel receiving portion is sized and shaped to receive at least a portion of an individual second channel forming unit, wherein the plurality of second channel receiving portions are constructed and arranged in a second pattern such that the plurality of second channel forming units can be assembled with the holder assembly to form a predetermined second array and the remaining space, and wherein the remaining space is configured to receive a hydrogel composition to form a solidified, hydrogel portion, such that a plurality of second channels in the predetermined, second array are formed by removing the plurality of second channel forming units from the hydrogel portion, individual second channel is configured to receive at least one second cell composition therein to form at least one second cell culture.

Embodiment 36. The device of embodiment 35, further comprising a plurality of third channel forming units to form a plurality of third channels for perfusion and optionally for receiving at least one third cells therein.

Embodiment 37. The device of any one of the embodiments 35-36, wherein the first diameter and the second diameter are larger than diameters of target first cell culture and target second cell culture, respectively.

Embodiment 38. The device of any one of embodiments 35-37, wherein the cultured tissue is cultured meat, the first composition comprises a plurality of first cells that are muscle derived cells, muscle satellite cells and/or myoblast derived cells; and the second composition, if present, comprises a plurality of second cells that are fat derived cells.

Embodiment 39. The device of any one of embodiments 34-38, wherein the first diameter is about 0.1-10,000 μm, for example, about 20-500 μm.

Embodiment 40. The device of any one of embodiments 34-39, wherein the first diameter is less than 200 μm, for example, 20-90 μm.

Embodiment 41. The device of any one of embodiments 34-40, wherein a distance between two adjacent first channel forming units is about 0.1-5,000 μm, for example, 0.1-500 μm.

Embodiment 42. The device of embodiment 41, wherein the distance between two adjacent first channel forming units is less than 100 μm, for example, 0.1-30 μm.

Embodiment 43. The device of any one of embodiments 35-42, wherein the second diameter is about 0.1-10,000 μm, for example, about 20-500 μm.

Embodiment 44. The device of any one of embodiments 35-43, wherein the second diameter is less than 500 μm, for example, 100-300 μm.

Embodiment 45. The device of any one of embodiments 35-44, wherein a distance between two adjacent second channel forming units is about 0.1-5,000 μm, for example, 0.1-500 μm, for example, about 1 μm.

Embodiment 46. The device of embodiment 45, wherein the distance between two adjacent second channel forming units is less than 100 μm, for example, 0.1-30 μm.

Embodiment 47. The device of any one of embodiments 36-46, wherein the third cells comprise at least one or more of fibroblasts, endothelial cells, myoblast cells, muscle derived cells, fat derived cells, and combination thereof.

Embodiment 48. The device of any one of embodiments 36-47, wherein individual first channel forming unit, second channel forming unit and/or third channel forming unit, if present, has a generally cylindrical, elongated structure.

Embodiment 49. The device of any one of embodiments 36-48, wherein individual first channel forming unit, second channel forming unit and/or third channel forming unit, if present, has an open, trough-like structure with a generally U-shaped cross-section.

Embodiment 50. The device of any one of embodiments 34-49, wherein the tissue construct comprises a plurality of fat regions and a plurality of muscle regions.

Embodiment 51. A system of producing cultured tissue, comprising: a device as claimed in any one of the embodiments 34-50 to produce a hydrogel construct having a plurality of third channels; a mixer for oxygenating a medium; a hydrogel construct adapter, configured to connect the hydrogel construct with the mixer; at least one pump system for circulating the medium from the mixer to the hydrogel construct through the plurality of third channels; and optionally a medium recycler to remove any waste.

Embodiment 52. The system of embodiment 51, wherein the cultured tissue is cultured meat, the first cell composition comprises a plurality of first cells that are muscle derived cells, muscle satellite cells and/or myoblast derived cells; and the second cell composition, if present, comprises a plurality of second cells that are fat derived cells.

Embodiment 53. A cultured tissue prepared by any one of the methods of embodiments 1-33, wherein the cultured tissues are derived from animals selected from the group consisting of mammals, birds, fish, invertebrates, reptiles, and amphibians.

Embodiment 54. The cultured tissue of embodiment 53, wherein the animals are non-human (e.g., porcine, sheep, cow, chicken).

Embodiment 55. The cultured tissue of embodiment 53, wherein the cultured tissue is cultured meat.

Embodiment 56. The cultured tissue of embodiment 53, wherein the animals are human.

1. A method of producing cultured tissue, comprising the steps of:
(a) providing a hydrogel composition and solidifying the hydrogel composition to form a hydrogel portion, wherein the hydrogel portion comprises a plurality of first channels in a predetermined first array;
(b) providing at least one first cell composition to individual first channel to form at least one first cell culture therein, such that a tissue construct is formed.

2. The method of claim 1, wherein the hydrogel portion further comprises a plurality of second channels in a predetermined second array, and the method further comprises the step of: (c) providing at least one second cell composition to individual second channel to form at least one second cell culture therein.

3. The method of claim 1, wherein the cultured tissue is cultured meat, the first cell composition comprises a plurality of first cells that are muscle derived cells, muscle satellite cells and/or myoblast derived cells; and the second cell composition, if present, comprises a plurality of second cells that are fat derived cells.

4. The method of claim 1, wherein the hydrogel composition comprises a first compound selected from a group consisting of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combinations thereof, and optionally a second compound selected from a group consisting of transglutaminase, thrombin, tyrosinase, and combinations thereof;
wherein the hydrogel composition further comprises fat derived cells and fibroblast cells, and combinations thereof.

5. The method of claim 1, wherein the hydrogel composition comprises any one of the following formulations:
type 1 collagen solution of about 0.1-20 mg/ml;
gelatin of about 0.5-50% (w/v) and gellan gum of about 0.01-5% (w/v);
gelatin of about 0.5-50% (w/v) and sodium alginate of about 0.1-6% (w/v);
gelatin of about 0.5-50% (w/v) and transglutaminase of about 0.1-100 U per gram of gelatin; and/or
fibrinogen of about 0.01-50 mg/mL and thrombin of about 0.01-100 U/mL.

6. (canceled)

7. The method of claim 2, wherein the at least one first cell composition comprises muscle derived cells, muscle satellite cells and/or myoblast derived cells, and optionally one or more of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combinations thereof; and wherein the at least one second cell composition comprises fat derived cells, and optionally one or more of collagen, alginate, gelatin, gellan gum, fibrinogen, xanthan gum, cellulose, plant protein, chitosan, carrageenan, starch hydrogels, agarose, pectin, guar gum, konjac glucomannan, lignin based hydrogels, and combinations thereof.

8. (canceled)

9. The method of claim 2, wherein the hydrogel portion further comprises a plurality of third channels as perfusion channels, and the method optionally further comprises the step of: (d) providing at least one third cell composition to individual third channel to form at least one third cell culture therein;
wherein the third cell composition comprises fibroblasts, endothelial cells, smooth muscle cells, pericytes, and combinations thereof.

10. (canceled)

11. The method of claim 9, wherein the plurality of first cells, second cells and/or third cells, if present, are derived from fibroblasts, endothelial cells, blood vessel cells, myoblast cells, muscle cells, fat cells, skin cells, tendon cells, liver cells, brain cells, bone cells, heart cells, kidney cells, nerve cells, stem cells, and combinations thereof.

12. The method of claim 9, wherein the at least one first cell composition, the at least one second cell composition and/or the at least one third cell composition, if present, further comprise any one of the following formulations:
type 1 collagen solution of about 0.1-20 mg/ml;
gelatin of about 0.5-50% (w/v) and gellan gum of about 0.01-5% (w/v);
gelatin of about 0.5-50% (w/v) and sodium alginate of about 0.1-6% (w/v);
gelatin of about 0.5-50% (w/v) and transglutaminase of about 0.1-100 U per gram of gelatin;
and/or fibrinogen of about 0.01-50 mg/mL and thrombin of about 0.01-100 U/mL.

13. The method of claim 12, wherein the hydrogel composition, or the at least one first cell composition, the at least one second cell composition and/or the at least one third cell composition, if present, comprise any one of the following formulations:
type 1 collagen solution of about 6 mg/ml;
gelatin of about 20% (w/v) and gellan gum of about 2% (w/v);
gelatin of about 20% (w/v) and sodium alginate of about 2% (w/v);
gelatin of about 13.5% (w/v) and transglutaminase of about 1 U per gram of gelatin; and/or
fibrinogen of about 1 mg/mL and thrombin of about 1 U/mL.

14. The method of claim 1, wherein the individual first channel and/or individual second channel, if present, are formed by the following steps:
(1) providing a device of producing cultured tissue comprising a plurality of first channel forming units, and/or second channel forming units and a holder assembly comprising:
a holder unit; and
at least one supporting unit, each comprising a plurality of first channel receiving portions, wherein individual first channel receiving portion is sized and shaped to receive at least a portion of an individual first channel forming unit, and wherein the plurality of first channel receiving portions are constructed and arranged in a first pattern such that the plurality of first channel forming units can be assembled with the holder assembly to form a predetermined first array and a remaining space in the holder assembly;
(2) providing a hydrogel composition to at least a portion of the remaining space and solidifying the hydrogel composition to form a hydrogel portion; and
(3) removing individual first channel forming unit and/or second channel forming unit from the hydrogel portion.

15. The method of claim 14, prior to step (2), further comprising the following steps:
(i) providing a second sacrificial layer in the holder assembly;
(ii) providing a third sacrificial layer on the second sacrificial layer;
(iii) providing a fourth sacrificial layer on the third sacrificial layer;
(iv) removing the third sacrificial layer, such that the second sacrificial layer and the fourth sacrificial layer defines the remaining space for hydrogel portion within the holder assembly.

16. The method of claim 15, prior to step (i), further comprising the step of:
providing a first sacrificial layer in the holder assembly, such that the second sacrificial layer is provided on the first sacrificial layer.

17. The method of claim 15, wherein step (i) further comprises the steps of: disposing the supporting unit at a first position proximate to the second sacrificial layer; and
wherein step (iii) further comprising step of: disposing the supporting unit at a second position proximate to the fourth sacrificial layer.

18. The method of claim 15, wherein the first sacrificial layer, the second sacrificial layer, the third sacrificial layer, and/or the fourth sacrificial layer comprises ice and/or hydrogel.

19-20. (canceled)

21. The method of claim 1, further comprising the step of: (e) growing the plurality of first cells, the plurality of second cells and/or the plurality of third cells, if present, until a desired tissue mass is obtained; wherein the step (e) is performed after step (b) or step (c), and wherein the plurality of third channels are formed by extruding out at least a portion of the hydrogel portion as perfusion channels by a plurality of third channel forming units.

22-24. (canceled)

25. The method of claim 1, further comprising the step of: (f) treating the tissue construct with a hydrogel shrinking agent.

26-29. (canceled)

30. The method of claim 3, wherein the fat derived cells are derived from adipose derived stem cells from cow; and wherein the muscle derived cells are derived from myoblast derived stem cells or muscle satellite cells from cow.

31. (canceled)

32. The method of claim 3, further comprising the step of: differentiating the first cells, the second cells and/or the third cells, if present, into mature, cell tissues.

33. The method of claim 3, wherein the first cell composition comprises porcine myoblast cells.

34-56. (canceled)
